(19) United States
(12) Patent Application Publication
(10) Pub. No.: US 2025/0186123 A1
(43) Pub. Date: Jun. 12, 2025

(54) INTRAOPERATIVE ALIGNMENT ASSESSMENT SYSTEM AND METHOD

(71) Applicant: GLOBUS MEDICAL, INC., AUDUBON, PA (US)

(72) Inventors: David Michael Gullotti, Towson, MD (US); Amir Hossein Soltanianzadeh, Baltimore, MD (US); Nicholas Theodore, Ruxton, MD (US); Edward Frederick Ruppel, III, Saratoga, MD (US); Saki Fujita, Baltimore, MD (US); Nicholas Griesmer Franconi, Pittsburgh, PA (US); Jennifer Lin, Oceanside, NY (US); Robert Li, Cypress, CA (US); Ali Uneri, Baltimore, MD (US); Sritam Parashar Rout, Dracut, MA (US); Marc Chelala, Montreal (CA); Kyle Robert Cowdrick, Lilburn, GA (US); Maria Fernanda Torres, Caracas (VE)

(21) Appl. No.: 19/050,601

(22) Filed: Feb. 11, 2025

Related U.S. Application Data
(63) Continuation of application No. 16/926,390, filed on Jul. 10, 2020.
Application No. 16/926,390 is a continuation-in-part of application No. 16/237,443, filed on Dec. 31, 2018, now Pat. No. 11,540,767.
Application No. 16/237,443 is a continuation-in-part of application No. 16/026,754, filed on Jul. 3, 2018, now abandoned.
(60) Provisional application No. 62/528,390, filed on Jul. 3, 2017.
(60) Provisional application No. 62/884,032, filed on Aug. 7, 2019.

(51) Int. Cl. A61B 34/10; A61B 34/00; A61B 90/00; G16H 10/60; G16H 50/30

(52) U.S. Cl. CPC A61B 34/10; A61B 34/25; A61B 90/39; G16H 10/60; G16H 50/30; A61B 2034/102; A61B 2034/105; A61B 2090/3966

(57) ABSTRACT

Some embodiments provide systems, assemblies, and methods of analyzing patient anatomy, including providing an analysis of a patient's spine, and also analyzing the biomechanical effects of implants. In some embodiments, the systems, assemblies, and/or methods can include obtaining initial patient data, acquiring spinal alignment and contour information, acquiring flexibility and/or biomechanical information, registering patient anatomical landmarks of interest relative to fiducial markers, analyzing databases of measurements and patient data to predict postoperative patient outcomes. Further, in some embodiments, the systems, assemblies, and/or methods can assess localized anatomical features of the patient, and obtain anatomical region data. In some embodiments, the systems, assemblies, and/or methods can also analyze the localized anatomy and therapeutic device location and contouring. Further, the systems, assemblies, and/or methods can output localized anatomical analyses and therapeutic device contouring data and/or imagery on a display according to some embodiments.

### RELATED APPLICATIONS

This application is a continuation of U.S. patent application Ser. No. 16/926,390, filed on Jul. 10, 2020, which is a continuation-in-part application of U.S. patent application Ser. No. 16/237,443, filed on Dec. 31, 2018, which is a continuation-in-part of U.S. patent application Ser. No. 16/026,754, filed Jul. 3, 2018, which claims priority to U.S. Provisional Patent Application No. 62/528,390, filed on Jul. 3, 2017. U.S. patent application Ser. No. 16/926,390 claims priority to U.S. Provisional Patent Application No. 62/884,032, filed Aug. 7, 2019. The entire contents of the aforementioned applications are incorporated herein by reference in their entireties for all purposes.

### BACKGROUND

Current tools limit a surgeon's ability to quickly and accurately assess the intraoperative alignment of their patient's spine, especially after the spine has been manipulated during a correction. In addition, most of the state-of-the-art options introduce or rely on excessive radiation exposure, inadequate visualization of anatomical landmark(s) of interest, and lengthy disruptions to the surgical workflow.

Accordingly, new systems and methods are needed analyzing and providing a patient's spinal alignment information and therapeutic device data. The method ideally should include obtaining initial patient data, and acquiring spinal alignment contour information, assessing localized anatomical features of the patient, and obtaining anatomical region data. The system and method should include analyzing the localized anatomy and therapeutic device location and contouring resulting in an output including a localized anatomical analysis and a display of therapeutic device contouring data.

### SUMMARY

Some embodiments include a system comprising at least one dynamic reference frame (DRF) configured so that any fixed or mobile portion of the DRF, or any assembly or component coupled to the DRF can be registered in 3D space using a plurality of trackable markers. In some embodiments, the plurality of trackable markers includes at least one moveable or triggerable marker. Some embodiments include at least one user-actuation trigger or actuator coupled to the at least one moveable or triggerable marker that can trigger or actuate the at least one moveable or triggerable marker. Some further embodiments include at least one 3D tracking camera or imaging system configured to track one or more of the plurality of trackable markers. In some embodiments, the system includes a processor and a memory coupled to the processor, wherein the memory stores instructions executable by the processor to track one or more 3D coordinates of one or more of the plurality of trackable markers.

In some embodiments of the method, the dynamic reference frame is attached to the patient. In some embodiments of the method, the dynamic reference frame is coupled to a surgical table or adjacent surface, where the dynamic reference frame is adjacent to the patient.

Some embodiments include a method of analyzing and providing a patient's spinal alignment information and therapeutic device data. In some embodiments, the method can comprise obtaining initial patient data, and acquiring spinal alignment contour information. In some embodiments, the method can comprise assessing localized anatomical features of the patient, and obtaining anatomical region data. In some embodiments, the method can include analyzing the localized anatomy and therapeutic device location and contouring. In some embodiments, the method can output localized anatomical analyses and therapeutic device contouring data on a display.

### DESCRIPTION OF THE DRAWINGS

FIG. 1A illustrates a perspective view of a skin-mounted fiducial assembly in accordance with some embodiments of the invention.

FIG. 1B illustrates a side view of a disengaged skin-mounted fiducial assembly as described previously in relation to FIG. 1A in accordance with some embodiments of the invention.

FIG. 1C illustrates a side view of an engaged skin-mounted fiducial assembly as described previously in relation to FIGS. 1A-1B in accordance with some embodiments of the invention.

FIG. 1D illustrates a side, cross-sectional view of an engaged skin-mounted fiducial assembly with an embedded radiopaque sphere as described previously in relation to FIGS. 1A-1C in accordance with some embodiments of the invention.

FIG. 1E illustrates a bottom view of the top skin-mounted fiducial with five asymmetrically distributed holes for embedding radiopaque spheres as described previously in relation to FIGS. 1A-1D in accordance with some embodiments of the invention.

FIG. 1F illustrates a cross-sectional view of the top skin-mounted fiducial as described previously in relation to FIGS. 1A-1E in accordance with some embodiments of the invention.

FIG. 1G illustrates an exploded view of the top skin-mounted fiducial, radiopaque spheres, a surgical drape, and bottom skin-mounted fiducial as described previously in relation to FIGS. 1A-IF in accordance with some embodiments of the invention.

FIG. 1H illustrates an exploded view of the top skin-mounted fiducial with embedded radiopaque spheres, a surgical drape, and bottom skin-mounted fiducial as described previously in relation to FIGS. 1A-1G in accordance with some embodiments of the invention.

FIG. 1I illustrates a perspective view of the skin-fiducial assembled over the surgical drape and disengaged as described previously in relation to FIGS. 1A-1H in accordance with some embodiments of the invention.

FIG. 1J illustrates a perspective view of a 3D-tracked tool with a tool ball-tip adapter engaged with the “Z-pattern” of the skin-mounted fiducial assembly as described previously in relation to FIGS. 1A-1I in accordance with some embodiments of the invention.

FIG. 2A illustrates a perspective view of an internal-mating bone-mounted fiducial in accordance with some embodiments of the invention.

FIG. 2B illustrates a top view of an internal-mating bone-mounted fiducial as described previously in relation to FIG. 2A in accordance with some embodiments of the invention.

FIG. 2C illustrates perspective views of an internal-mating bone-mounted fiducial and external-mating tool tip adapter of a 3D-tracked tool as described previously in relation to FIGS. 2A-2B in accordance with some embodiments of the invention.

FIG. 2D illustrates perspective views of a 3D-tracked tool with an external-mating tool tip adapter not engaged with an internal-mating bone-mounted fiducial as described previously in relation to FIGS. 2A-2C in accordance with some embodiments of the invention.

FIG. 2E illustrates perspective views of a 3D-tracked tool with an external-mating tool tip adapter engaged with an internal-mating bone-mounted fiducial as described previously in relation to FIGS. 2A-2D in accordance with some embodiments of the invention.

FIG. 2F illustrates perspective views of a 3D-tracked tool with an external-mating tool tip adapter not engaged with an internal-mating bone-mounted fiducial implanted into the sacrum and in an untriggered state as described previously in relation to FIGS. 2A-2E in accordance with some embodiments of the invention.

FIG. 2G illustrates perspective views of a 3D-tracked tool with an external-mating tool tip adapter engaged with an internal-mating bone-mounted fiducial implanted into the sacrum and in a triggered state as described previously in relation to FIGS. 2A-2F in accordance with some embodiments of the invention.

FIG. 2H illustrates a coronal plane view of the spine with bone-mounted fiducials implanted into the sacrum and several laminae as described in relation to FIGS. 2A-2G in accordance with some embodiments of the invention.

FIG. 2I illustrates perspective views of a 3D-tracked tool with an external-mating tool tip adapter not engaged with internal-mating bone-mounted fiducials implanted into the sacrum and laminae and in an untriggered state as described previously in relation to FIGS. 2A-2H in accordance with some embodiments of the invention.

FIG. 2J illustrates perspective views of a 3D-tracked tool with an external-mating tool tip adapter engaged with internal-mating bone-mounted fiducials implanted into the sacrum and laminae and in a triggered state as described previously in relation to FIGS. 2A-2I in accordance with some embodiments of the invention.

FIG. 3A illustrates a perspective view of an external-mating bone-mounted fiducial in accordance with some embodiments of the invention.

FIG. 3B illustrates a top view of an external-mating bone-mounted fiducial as described previously in relation to FIG. 3A in accordance with some embodiments of the invention.

FIG. 3C illustrates perspective views of an external-mating bone-mounted fiducial and internal-mating tool tip adapter of a 3D-tracked tool as described previously in relation to FIGS. 3A-3B in accordance with some embodiments of the invention.

FIG. 3D illustrates a front view of a 3D-tracked tool with an internal-mating tool tip adapter not engaged with an external-mating bone-mounted fiducial and in an untriggered state as described previously in relation to FIGS. 3A-3C in accordance with some embodiments of the invention.

FIG. 3E illustrates frontal views of a 3D-tracked tool with an internal-mating tool tip adapter engaged with an external-mating bone-mounted fiducial and in an untriggered state as described previously in relation to FIGS. 3A-3D in accordance with some embodiments of the invention.

FIG. 3F illustrates frontal views of a 3D-tracked tool with an internal-mating tool tip adapter engaged with an external-mating bone-mounted fiducial and in a triggered state as described previously in relation to FIGS. 3A-3E in accordance with some embodiments of the invention.

FIG. 3G illustrates perspective views of a 3D-tracked tool with an internal-mating tool tip adapter not engaged with an external-mating bone-mounted fiducial and in an untriggered state as described previously in relation to FIGS. 3A-3F in accordance with some embodiments of the invention.

FIG. 3H illustrates perspective views of a 3D-tracked tool with an internal-mating tool tip adapter engaged with an external-mating bone-mounted fiducial and in an untriggered state as described previously in relation to FIGS. 3A-3G in accordance with some embodiments of the invention.

FIG. 3I illustrates perspective views of a 3D-tracked tool with an internal-mating tool tip adapter engaged with an external-mating bone-mounted fiducial and in a triggered state as described previously in relation to FIGS. 3A-3H in accordance with some embodiments of the invention.

FIG. 4A illustrates a top perspective view of an external-mating bone-mounted fiducial attached to a single-screw plate in accordance with some embodiments of the invention.

FIG. 4B illustrates a bottom perspective view of an external-mating bone-mounted fiducial attached to a single-screw plate as described previously in relation to FIG. 4A in accordance with some embodiments of the invention.

FIG. 4C illustrates a top view of an external-mating bone-mounted fiducial attached to a single-screw plate as described previously in relation to FIGS. 4A-4B in accordance with some embodiments of the invention.

FIG. 4D illustrates a side view of an external-mating bone-mounted fiducial attached to single-screw plate as described previously in relation to FIGS. 4A-4C in accordance with some embodiments of the invention.

FIG. 4E illustrates a front view of an external-mating bone-mounted fiducial attached to single-screw plate as described previously in relation to FIGS. 4A-4D in accordance with some embodiments of the invention.

FIG. 4F illustrates perspective views of an external-mating bone-mounted fiducial attached to a single-screw plate and an internal-mating tool tip adapter of a 3D-tracked tool as described previously in relation to FIGS. 4A-4E in accordance with some embodiments of the invention.

FIG. 4G illustrates a front view of a 3D-tracked tool with internal-mating tool tip adapter not engaged with an external-mating bone-mounted fiducial attached to a single-screw plate and in an untriggered state as described previously in relation to FIGS. 4A-4F in accordance with some embodiments of the invention.

FIG. 4H illustrates a side view of a 3D-tracked tool with internal-mating tool tip adapter engaged with an external-mating bone-mounted fiducial attached to a single-screw plate and in an untriggered state as described previously in relation to FIGS. 4A-4G in accordance with some embodiments of the invention.

FIG. 4I illustrates a side view of a 3D-tracked tool with internal-mating tool tip adapter engaged with an external-mating bone-mounted fiducial attached to a single-screw plate and in a triggered state as described previously in relation to FIGS. 4A-4H in accordance with some embodiments of the invention.

FIG. 4J illustrates a perspective view of a 3D-tracked tool with internal-mating tool tip adapter not engaged with an external-mating bone-mounted fiducial attached to a single-screw plate and in an untriggered state as described previously in relation to FIGS. 4A-4I in accordance with some embodiments of the invention.

FIG. 4K illustrates a perspective view of a 3D-tracked tool with internal-mating tool tip adapter engaged with an external-mating bone-mounted fiducial attached to a single-screw plate and in a triggered state as described previously in relation to FIGS. 4A-4J in accordance with some embodiments of the invention.

FIG. 5A illustrates a top perspective view of an internal-mating bone-mounted fiducial attached to a single-screw plate in accordance with some embodiments of the invention.

FIG. 5B illustrates a side view of an internal-mating bone-mounted fiducial as described previously in relation to FIG. 5A in accordance with some embodiments of the invention.

FIG. 5C illustrates a top view of an internal-mating bone-mounted fiducial as described previously in relation to FIGS. 5A-5B in accordance with some embodiments of the invention.

FIG. 5D illustrates perspective views of an internal-mating bone-mounted fiducial attached to single-screw plate and an external-mating tool tip adapter of a 3D-tracked tool as described previously in relation to FIGS. 5A-5C in accordance with some embodiments of the invention.

FIG. 5E illustrates perspective views of an internal-mating bone-mounted fiducial attached to a single-screw plate engaged with an external-mating tool tip adapter of a 3D-tracked tool as described previously in relation to FIGS. 5A-5D in accordance with some embodiments of the invention.

FIG. 5F illustrates a perspective view of a 3D-tracked tool with external-mating tool tip adapter disengaged with an internal-mating bone-mounted fiducial attached to a single-screw plate and in an untriggered state as described previously in relation to FIGS. 5A-5E in accordance with some embodiments of the invention.

FIG. 5G illustrates a perspective view of a 3D-tracked tool with external-mating tool tip adapter engaged with an internal-mating bone-mounted fiducial attached to a single-screw plate and in an untriggered state as described previously in relation to FIGS. 5A-5F in accordance with some embodiments of the invention.

FIG. 5H illustrates a perspective view of a 3D-tracked tool with external-mating tool tip adapter engaged with an internal-mating bone-mounted fiducial attached to a single-screw plate and in a triggered state as described previously in relation to FIGS. 5A-5G in accordance with some embodiments of the invention.

FIG. 6A illustrates a perspective view of an external-mating bone-mounted fiducial X-Ray adapter with asymmetrically distributed holes for embedding radiopaque spheres in accordance with some embodiments of the invention.

FIG. 6B illustrates a front view of an external-mating bone-mounted fiducial X-Ray adapter with asymmetrically distributed holes for embedding radiopaque spheres as described previously in relation to FIG. 6A in accordance with some embodiments of the invention.

FIG. 6C illustrates a side view of an external-mating bone-mounted fiducial X-Ray adapter with asymmetrically distributed holes for embedding radiopaque spheres as described previously in relation to FIGS. 6A-6B in accordance with some embodiments of the invention.

FIGS. 6D-6F illustrate perspective views of an external-mating bone-mounted fiducial X-Ray adapter embedded with asymmetrically distributed radiopaque spheres disengaged and engaged with an internal-mating bone-mounted fiducial implanted into the sacrum as described previously in relation to FIGS. 6A-6C in accordance with some embodiments of the invention.

FIG. 6G illustrates an X-Ray image taken from a lateral view visualizing the pelvis and asymmetrically distributed radiopaque spheres of the bone-mounted fiducial X-Ray adapter as described previously in relation to FIGS. 6A-6F in accordance with some embodiments of the invention.

FIG. 6H illustrates an X-Ray image taken from an AP view visualizing the pelvis and asymmetrically distributed radiopaque spheres of the bone-mounted fiducial X-Ray adapter as described previously in relation to FIGS. 6A-6G in accordance with some embodiments of the invention.

FIGS. 7A-7B illustrate top and bottom perspective views of an internal-mating bone-mounted fiducial X-Ray adapter with asymmetrically distributed holes for embedding radiopaque spheres and arrow indicator for determining anatomical axes and an external-mating bone-mounted fiducial in accordance with some embodiments of the invention.

FIG. 7C illustrates a perspective view of an internal-mating bone-mounted fiducial X-Ray adapter embedded with asymmetrically distributed radiopaque spheres disengaged with an external-mating bone-mounted fiducial implanted into the sacrum as described previously in relation to FIGS. 7A-7B in accordance with some embodiments of the invention.

FIG. 7D illustrates a top view of an internal-mating bone-mounted fiducial X-Ray adapter embedded with asymmetrically distributed radiopaque spheres engaged with an external-mating bone-mounted fiducial implanted into the sacrum as described previously in relation to FIGS. 7A-7C in accordance with some embodiments of the invention.

FIG. 8A illustrates a perspective view of an external-mating bone-mounted fiducial X-Ray adapter with three symmetrically distributed holes for embedding radiopaque spheres with an arrow indicator for determining anatomical axes in accordance with some embodiments of the invention.

FIG. 8B illustrates a front view of an external-mating bone-mounted fiducial X-Ray adapter with three symmetrically distributed holes for embedding radiopaque spheres with an arrow indicator for determining anatomical axes as described previously in relation to FIG. 8A in accordance with some embodiments of the invention.

FIG. 8C illustrates a side view of an external-mating bone-mounted fiducial X-Ray adapter with three symmetrically distributed holes for embedding radiopaque spheres with an arrow indicator for determining anatomical axes as described previously in relation to FIGS. 8A-8B in accordance with some embodiments of the invention.

FIG. 8D illustrates a side cross-sectional view of an external-mating bone-mounted fiducial X-Ray adapter with three symmetrically distributed radiopaque spheres embedded as described previously in relation to FIGS. 8A-8C in accordance with some embodiments of the invention.

FIG. 8E illustrates a perspective view of an external-mating bone-mounted fiducial X-Ray adapter with three symmetrically distributed radiopaque spheres engaged with an internal-mating bone-mounted fiducial implanted in the sacrum as described previously in relation to FIGS. 8A-8D in accordance with some embodiments of the invention.

FIG. 9A illustrates a perspective view of a bone-mounted fiducial with a star-shaped internal-mating mechanism in accordance with some embodiments of the invention.

FIG. 9B illustrates a top view of a bone-mounted fiducial with a star-shaped internal-mating mechanism as described previously in relation to FIG. 9A in accordance with some embodiments of the invention.

FIG. 9C illustrates a side view of a bone-mounted fiducial with a star-shaped internal-mating mechanism as described previously in relation to FIGS. 9A-9B in accordance with some embodiments of the invention.

FIG. 9D illustrates a perspective view of a 3D-tracked tool with external-mating tool tip adapter disengaged with an internal-mating bone-mounted fiducial implanted in the vertebra as described previously in relation to FIGS. 9A-9C in accordance with some embodiments of the invention.

FIGS. 9E-9F illustrate a perspective view of a 3D-tracked tool with external-mating tool tip adapter engaged with an internal-mating bone-mounted fiducial implanted in the vertebra and in an untriggered and triggered state as described previously in relation to FIGS. 9A-9D in accordance with some embodiments of the invention.

FIG. 9G illustrates a perspective view of a 3D-tracked tool with external-mating tool tip adapter engaged with an internal-mating bone-mounted fiducial implanted in the vertebra and in an untriggered state as described previously in relation to FIGS. 9A-9F in accordance with some embodiments of the invention.

FIG. 9H illustrates a perspective view of a 3D-tracked tool with external-mating tool tip adapter engaged with an internal-mating bone-mounted fiducial implanted in the vertebra and in a triggered state as described previously in relation to FIGS. 9A-9G in accordance with some embodiments of the invention.

FIG. 10A illustrates a perspective view of a 3D-tracked tool with an external-mating tool tip not engaged with internal-mating bone-mounted fiducials in accordance with some embodiments of the invention.

FIG. 10B illustrates a perspective view of a 3D-tracked tool with an external-mating tool tip engaged in a unique orientation with an internal-mating bone-mounted fiducial and in a triggered state as described previously in relation to FIG. 10A in accordance with some embodiments of the invention.

FIG. 10C illustrates a side view of a 3D-tracked tool with an external-mating tool tip not engaged with internal-mating bone-mounted fiducials as described previously in relation to FIGS. 10A-10B in accordance with some embodiments of the invention.

FIG. 10D illustrates a side view of a 3D-tracked tool with an external-mating tool tip engaged in a unique orientation with an internal-mating bone-mounted fiducial and in a triggered state as described previously in relation to FIGS. 10A-10C in accordance with some embodiments of the invention.

FIG. 11A illustrates a perspective view of a rod contour registration tool with linear triggering mechanism in accordance with some embodiments of the invention.

FIG. 11B illustrates a side view of a rod contour registration tool disengaged with a rod and in an untriggered state as described previously in relation to FIG. 11A in accordance with some embodiments of the invention.

FIG. 11C illustrates a side view of a rod contour registration tool engaged with a rod and in a triggered state as described previously in relation to FIGS. 11A-11B in accordance with some embodiments of the invention.

FIG. 11D illustrates a cross-sectional view of a rod contour registration tool disengaged with a rod and in an untriggered state as described previously in relation to FIGS. 11A-11C in accordance with some embodiments of the invention.

FIG. 11E illustrates a cross-sectional view of a rod contour registration tool engaged with a rod and in a triggered state as described previously in relation to FIGS. 11A-11D in accordance with some embodiments of the invention.

FIG. 11F illustrates a perspective view of a rod contour registration tool attached to a rod bender as described previously in relation to FIGS. 11A-11E in accordance with some embodiments of the invention.

FIG. 11G illustrates a back view of a rod contour registration tool attached to a rod bender as described previously in relation to FIGS. 11A-11F in accordance with some embodiments of the invention.

FIG. 11H illustrates a side view of a rod contour registration tool attached to a rod bender as described previously in relation to FIGS. 11A-11G in accordance with some embodiments of the invention.

FIGS. 111-11J illustrate perspective views of a rod contour registration tool attached to a rod bender disengaged and engaged with a bent rod attached to a coordinate reference end cap device as described previously in relation to FIGS. 11A-11H in accordance with some embodiments of the invention.

FIG. 12A illustrates a front view of a front-facing flexibility assessment device in accordance with some embodiments of the invention.

FIG. 12B illustrates a top view of a front-facing flexibility assessment device as described previously in relation to FIG. 12A in accordance with some embodiments of the invention.

FIGS. 12C-12D illustrate perspective views of a front-facing and back-facing flexibility assessment devices as described previously in relation to FIG. 12A-12B in accordance with some embodiments of the invention.

FIG. 13A illustrates a top view of the adjustable pedicle screw interfaces of a flexibility assessment device rigidly engaged with vertebrae and spinal rods in accordance with some embodiments of the invention.

FIG. 13B illustrates a side view of the adjustable pedicle screw interfaces of a flexibility assessment device rigidly engaged with vertebrae as described previously in relation to FIG. 13A in accordance with some embodiments of the invention.

FIG. 13C illustrates a top view of the front-facing and back-facing flexibility assessment devices rigidly engaged with the adjustable pedicle screw interface and corresponding vertebrae as described previously in relation to FIGS. 13A-13B in accordance with some embodiments of the invention.

FIG. 13D illustrates a side view of the front-facing and back-facing flexibility assessment devices rigidly engaged with the adjustable pedicle screw interface and corresponding vertebrae as described previously in relation to FIGS. 13A-13B in accordance with some embodiments of the invention.

FIG. 14A illustrates a perspective view of a 3D-tracked tool engaged with a pedicle screw inserted in the lamina in accordance with some embodiments of the invention.

FIG. 14B illustrates perspective views of a 3D-tracked tool and one side of the adjustable pedicle screw interfaces of the flexibility assessment device attached rigidly to vertebrae as described previously in relation to FIG. 14A in accordance with some embodiments of the invention.

FIG. 14C illustrates perspective views of a 3D-tracked tool engaged with a pedicle screw inserted in lamina and one side of the adjustable pedicle screw interfaces of the flexibility assessment device attached rigidly to vertebrae as described previously in relation to FIGS. 14A-14B in accordance with some embodiments of the invention.

FIG. 14D illustrates a top view of a 3D-tracked tool engaged with a pedicle screw inserted in lamina and one side of the adjustable pedicle screw interfaces of the flexibility assessment device attached rigidly to vertebrae as described previously in relation to FIGS. 14A-14C in accordance with some embodiments of the invention.

FIG. 15A illustrates a perspective view of a 3D-tracked tool with a tool ball tip adapter engaged with the “Z-pattern” of the top skin-mounted fiducial attached on the skin covering the vertebrae in accordance with some embodiments of the invention.

FIG. 15B illustrates a perspective view of a 3D-tracked tool with a tool ball tip adapter tracing the laminae region of the vertebrae as described previously in relation to FIG. 15A in accordance with some embodiments of the invention.

FIG. 15C illustrates a top view of a 3D-tracked tool with a tool ball tip adapter tracing the sacrum region of the vertebrae as described previously in relation to FIGS. 15A-15B in accordance with some embodiments of the invention.

FIG. 15D illustrates a zoomed-out view of a 3D-tracked tool with a tool ball tip adapter engaged with the “Z-pattern” of the top skin-mounted fiducial attached on the skin covering the vertebrae as described previously in relation to FIGS. 15A-15C in accordance with some embodiments of the invention.

FIG. 15E illustrates a zoomed-out view of a 3D-tracked tool with a tool ball tip adapter tracing the laminae region of the vertebrae as described previously in relation to FIGS. 15A-15D in accordance with some embodiments of the invention.

FIG. 15F illustrates a zoomed-out view of a 3D-tracked tool with a tool ball tip adapter tracing the sacrum region of the vertebrae as described previously in relation to FIGS. 15A-15E in accordance with some embodiments of the invention.

FIG. 16A illustrates a perspective view of an external-mating bone-mounted fiducial and internal-mating bone-mounted fiducial X-Ray adapter with five asymmetrically distributed holes for embedding radiopaque spheres in accordance with some embodiments of the invention.

FIG. 16B illustrates a top view of an external-mating bone-mounted fiducial with five asymmetrically distributed holes for embedding radiopaque spheres as described previously in relation to FIG. 16A in accordance with some embodiments of the invention.

FIGS. 16C-16D illustrate side views of an external-mating bone-mounted fiducial and internal-mating bone-mounted fiducial X-Ray adapter with five asymmetrically distributed holes for embedding radiopaque spheres as described previously in relation to FIGS. 16A-16B in accordance with some embodiments of the invention.

FIG. 16E illustrates a bottom view of an external-mating bone-mounted fiducial and internal-mating bone-mounted fiducial X-Ray adapter with five asymmetrically distributed holes for embedding radiopaque spheres as described previously in relation to FIGS. 16A-16D in accordance with some embodiments of the invention.

FIG. 16F illustrates an X-Ray image taken from a lateral view visualizing the pelvis and five radiopaque spheres of the bone-mounted fiducial X-Ray adapter as described previously in relation to FIGS. 16A-16E in accordance with some embodiments of the invention.

FIG. 16G illustrates an X-Ray image taken from an AP view visualizing the pelvis and five radiopaque spheres of the bone-mounted fiducial X-Ray adapter as described previously in relation to FIGS. 16A-16F in accordance with some embodiments of the invention.

### DETAILED DESCRIPTION

In some embodiments, a system can allow a surgeon to make intraoperative assessments and adjustments of the patient's alignment and biomechanical abilities. In some embodiments, the system can register the patient's local and/or full-length spinal curvature and flexibility. In some embodiments, the system can register the instruments and/or implants used to assess and/or manipulate the conformation of the spine. In some embodiments, the system can use various calculations and algorithms to produce a quantitative assessment of the patient's spinal biomechanical qualities and the customized implants used to enhance these qualities. In some embodiments, these quantitative assessments can include, but are not limited to, calculated values for various radiographic parameters related to both global and segmental alignment of the spine (e.g., lumbar lordosis, central sacral vertical line, T1 pelvic angle, thoracic kyphosis, Cobb angle, etc.).

Some embodiments include key features. In some embodiments, one or more of the embodiments described herein can include anatomical landmark(s) of interest (e.g., C7, SI, etc.) that are initialized relative to the 3D-tracking acquisition system. In some embodiments, a continuous or discrete 3D-tracked acquisition can be made along the surface (e.g., posterior, anterior, or lateral) of the spine, both within and beyond the surgical site (e.g., skin surface). Some embodiments include a series of algorithms filter continuous or discrete 3D-tracked probe data to identify a relationship between the acquired points and anatomical regions of interest (e.g., centroids of the vertebral bodies). In some embodiments, an assessment of the segmental and/or full-length spinal alignment can be produced with values for each relevant radiographic parameter (e.g., Cobb angle, lumbar lordosis, thoracic kyphosis, C2-C7 lordosis, C7-S I sagittal vertical axis, central sacral vertical line, T1 pelvic angle, pelvic incidence, pelvic-incidence-lumbar-lordosis mismatch, etc.). In some embodiments, an assessment of the contour, position, and/or alignment of instrumented hardware, such as screws, rods, or cages, can be produced.

Some embodiments include a visual display and quantitative feedback system for assessing and adjusting implants that can be implanted into/onto the anatomy, including 3D, dynamic renderings of registered anatomical landmark(s) of interest. In some embodiments, an assessment of segmental, regional, or full-length flexibility and range of motion can be produced between a selected range of vertebral segments. In some embodiments, the display outputs the information about the spine's curvature and alignment, quantitative radiographic alignment parameter values, instrumented hardware analysis, flexibility or range of motion of the spine, and also various ways to acquire or analyze radiographic images. In some embodiments, the display enables interactive feedback and interfaces for the user to signal specific commands to the system for computing, beginning operations for, or outputting the quantitative or visual analysis of a system or anatomical region(s) of interest.

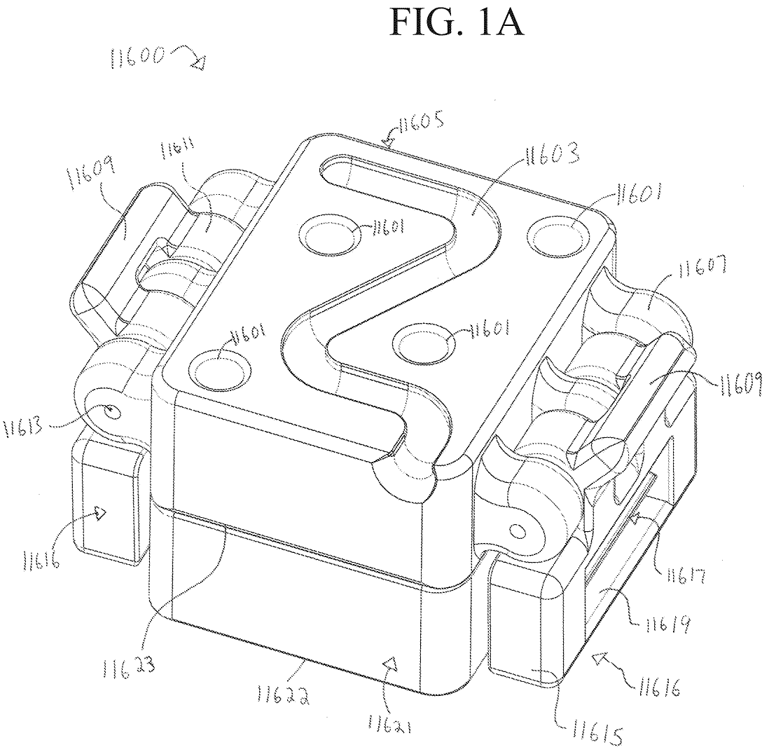

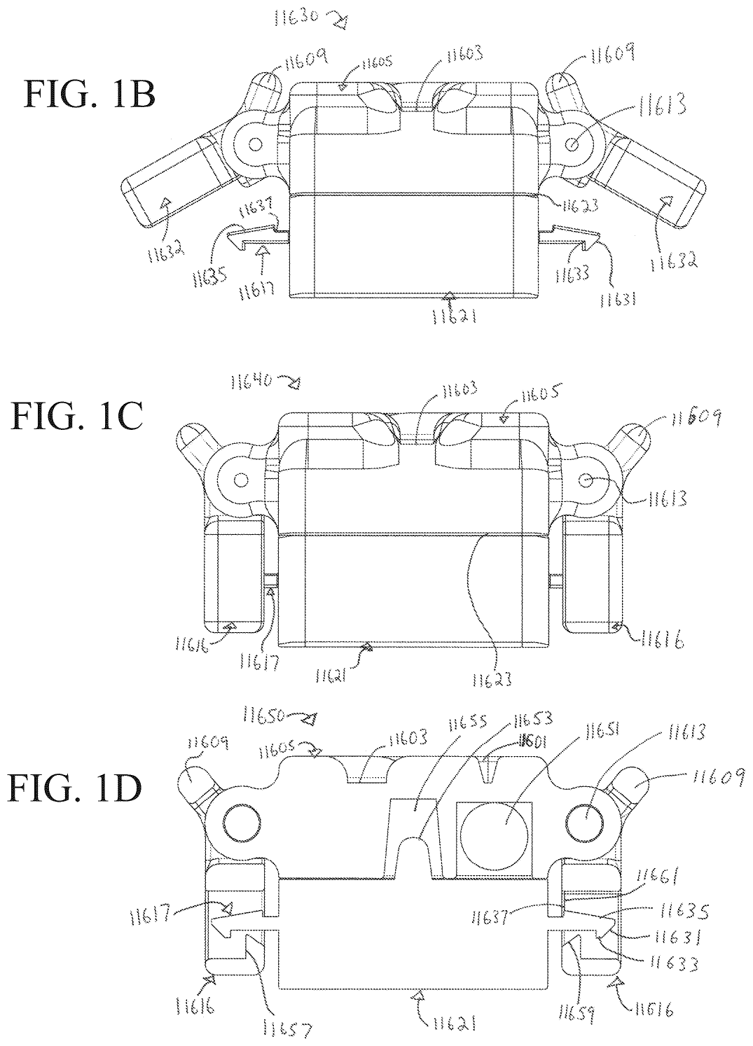

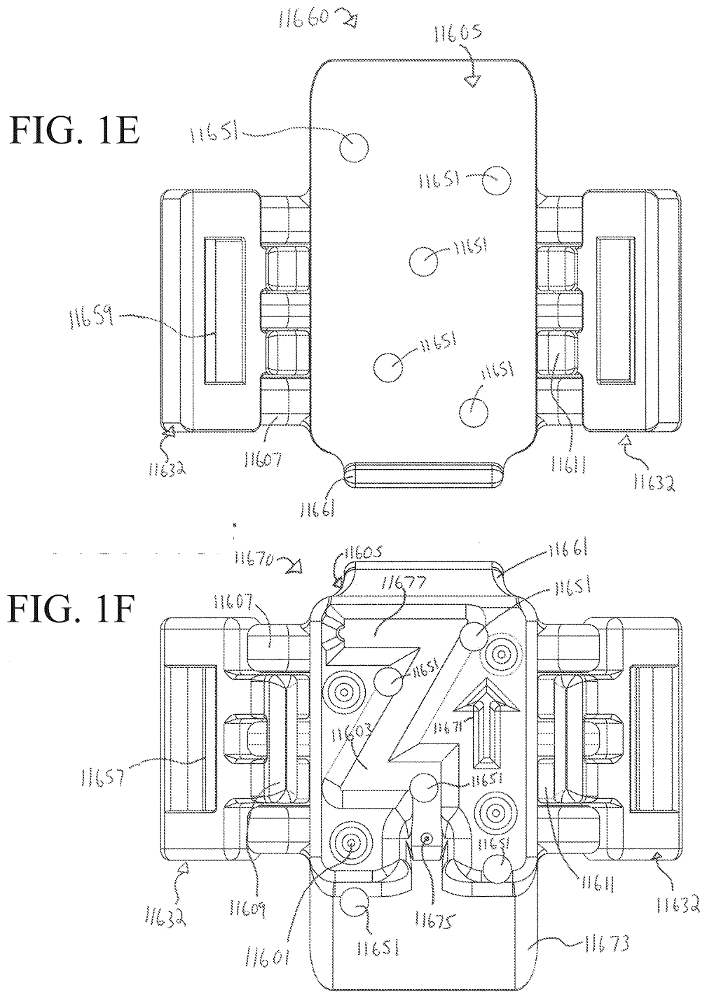

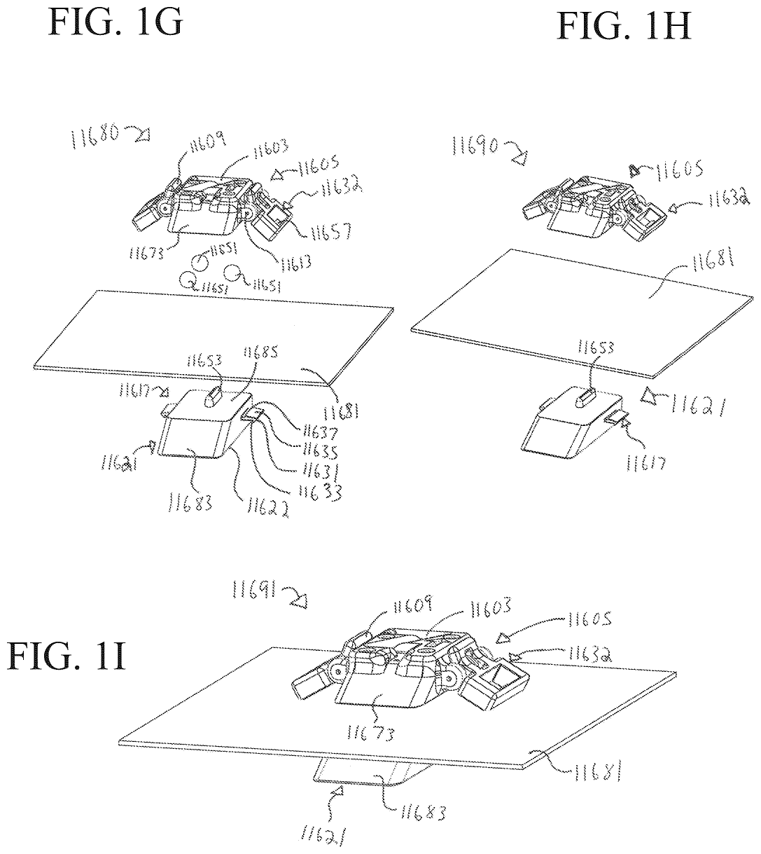

Some embodiments include a skin-mounted fiducial device for registering the 3D location and pose of key anatomical landmarks of interest outside the surgical site. In some embodiments, the locations of underlying anatomical landmarks can be used for calculations of spinal alignment parameters and other related biomechanical analyses. In some embodiments, FIG. 1A illustrates an assembly 100 for a skin-mounted, two-part fiducial device that comprises of a top-half fiducial 11605 and a bottom-half fiducial 11621 with an adhesive backing 11622. In some embodiments, the top-half fiducial 11605 features a surface tracing registration groove 11603 for rapid registration of the fiducial 3D coordinates axes in navigation camera coordinates via a 3D-tracked probe tracing within the groove. In some embodiments, the top-half fiducial 11605 contains an array of asymmetrically-arranged, embedded radiopaque fiducial markers 11651 for X-ray-based registration of the fiducial's 3D location and pose relative to underlying anatomical landmarks of interest.

In some embodiments, two or more multi-planar X-Ray images of the fiducial device are to be acquired with the anatomical landmarks of interest also visible in the same X-Ray images. In some embodiments, in each image, using the known asymmetric geometric distribution between radiopaque fiducial markers 11651, the system automatically calculates the 3D pose of the fiducial relative to the X-Ray imaging detector plane. In some embodiments, the user annotates the key anatomical landmarks of interest in each X-Ray image and then the system automatically computes the 3D locations of all annotated landmarks with respect to the fiducial centroid coordinates axes. In some embodiments, once the user registers the surface location and pose of the top-half fiducial 11605 with a tracing of the registration groove 11603 or tapping three or more detents 11601, the system then computes the location of all initialized anatomical landmarks with respect to the coordinate axes of the navigation camera, thus enabling computation of spinal alignment parameters with respect to 3D probe tracings or other registrations of spinal landmarks.

In some embodiments, since surgical drapes are applied frequently to the area surrounding the surgical site, the fiducial device is comprised of two complementary halves that mate across surgical drapes to enable for continued updates on the 3D location and pose of underlying anatomical landmarks of interest without additional X-Ray images. Some embodiments of the invention involve mechanical mating mechanisms to rigidly link the top-half fiducial 11605 to the bottom-half fiducial 11621. Some embodiments involve a winged clamping mechanism that features one or more side-clamps 11609 that revolve about a hinge joint 11607 via a pivot bearing 11613. In some embodiments, the side-clamp wall 11615 features a sloped mating surface 11619 that mechanically engages with a bottom fiducial protrusion mate 11617, and in doing so the pair of fiducial halves pinch across the surgical drape interface 11623 to enable a rigid, bump-resistant mate.

In some embodiments, FIG. 1B illustrates a side view 11630 of the fiducial device assembly described previously in relation to FIG. 1A in a disengaged position. In some embodiments, in this assembly the side-clamp walls 11632 are in an open position. In some embodiments, the bottom fiducial's clamping features include a series of upward and downward sloping mating surfaces that help mechanically retain the sloped surface of the side-clamp wall 11632 and its mating feature 11619. In some embodiments, FIG. 1C illustrates a side view 11640 of the fiducial device assembly described previously in relation to FIGS. 1A-B in an engaged, closed position with the intermediate surgical drape not shown. In some embodiments, FIG. 1D illustrates a side, cross-sectional view 11650 of the skin-mounted fiducial assembly. The primary mating features of the fiducial halves surrounding both sides of the surgical drape include the male alignment protrusion 11653 which is received by the female alignment groove 11655. In some embodiments, the side-clamp walls 11616 have a series of sloped mating surfaces 11659 that press against the downward sloped mating surface 11631 of the bottom-half fiducial's protrusion mates 11617.

In some embodiments, FIG. 1E illustrates a side view 11660 of the top-half fiducial 11605 device described previously in relation to FIGS. 1A-D in an open, disengaged position. In some embodiments, the underside of the top-half fiducial involves three or more radiopaque marker spheres in an asymmetric, unique arrangement, and in some embodiments one of the spheres is located at the centroid of the pattern. In some embodiments, by locating one sphere at the centroid of the pattern, greater accuracies can be achieved for registering the centroid and subsequent pose of the fiducial pattern in X-Ray images, since the imaged patterns are compared relative to a ground-truth coordinate database with spheres being rotated about the centroid of the fiducial. In some embodiments, FIG. 1F illustrates an assembly side view 11670 that demonstrate that the radiopaque fiducial spheres 11651 can be visualized in relation to the top surface of the fiducial. In some embodiments, when a 3D-tracked probe traces and registers the 3D coordinate axes of the top-half fiducial 11605, the system calculates where the centroid of the radiopaque fiducial spheres 11651 are with respect to the fiducial surface coordinates measured relative to the navigation camera. In some embodiments, using this registration of the fiducial sphere coordinates relative to the navigation camera, a 3D rigid body transform is computed to convert the 3D displacement vectors of the anatomical landmarks of interest measured relative to X-Ray fiducial coordinates to now be calculated in navigation camera coordinates.

In some embodiments, FIGS. 1G-1I illustrate exploded perspective views (11680, 11690, 11691) of the fiducial assembly device described previously in relation to FIGS. 1A-F. In some embodiments, these views illustrate the process of mating the top-half fiducial 11605 to the bottom-half fiducial 11621 across the surface of the surgical drape 11681 that sandwiches between both device halves.

In some embodiments, FIG. 1J illustrates an assembly view 11692 of a 3D-tracked probe 11682 that is engaged with the skin-mounted fiducial assembly described previously in relation to FIGS. 1A-I. In some embodiments, the 3D-tracked probe comprises a probe shaft 11679 encased in a trigger sleeve handle 11698, and a 3D-tracked dynamic reference frame (DRF) 11686 that comprises a unique array of 3D-tracked markers 11684 and is linked to the probe via a screw 11693 attaching the array to a DRF mount 11699 along the probe shaft 11679. In some embodiments, the 3D-tracked probe contains a tracked mobile stray marker (TMSM) 11695 that is substantially rigidly linked to a triggering spring-loaded link mechanism (not shown) that is actuated via a trigger button 11697. In some embodiments, the system automatically detects the TMSM's location within a known 3D volume boundary with respect to the location and pose of the DRF, and based on user-selected triggering sensitivity thresholds, the location of the TMSM beyond a set threshold signals an active probe state to the computer. In some embodiments, the TMSM 11695 is linked to the triggering mechanism via a backing mount 11696 that slides along a sliding mount backing 11667 via a spring-loaded (not shown) or otherwise conventionally biased screw sliding in a groove 11668 along the probe shaft axis. In some embodiments, the 3D-tracked probe 11682 contains a spherical probe tip 11666 that is used to both mate with fiducial devices initialized within and beyond the surgical site. In some embodiments, the spherical probe tip 11666 mates with the tracing registration groove 11603, and when the probe TMSM 11695, the system analyzes a 3D tracing of the groove pattern 11603 to compute the fiducial coordinate axes with respect to the navigation camera (not shown).

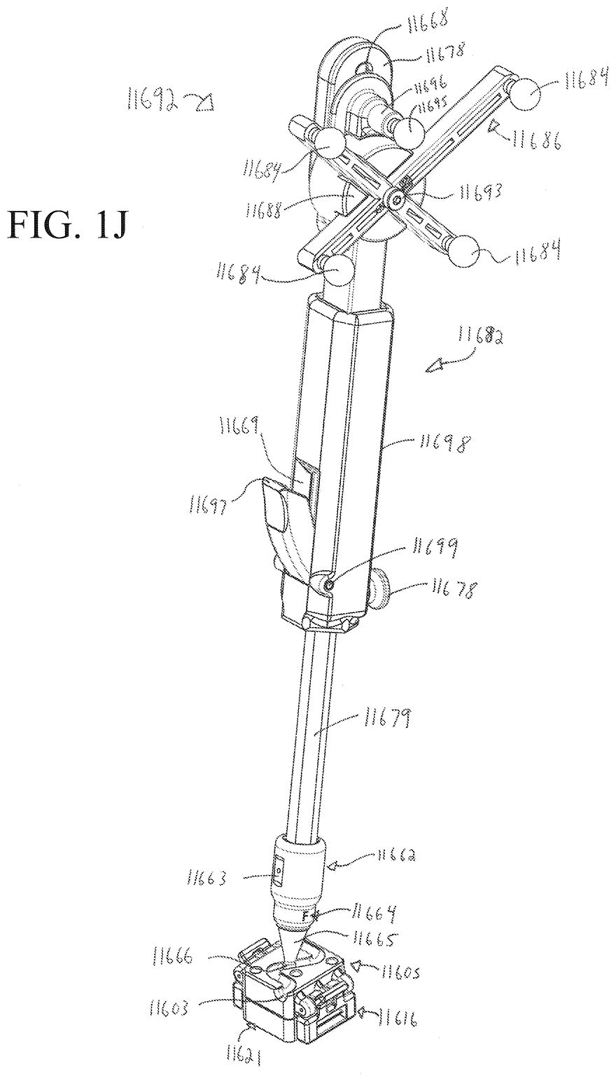

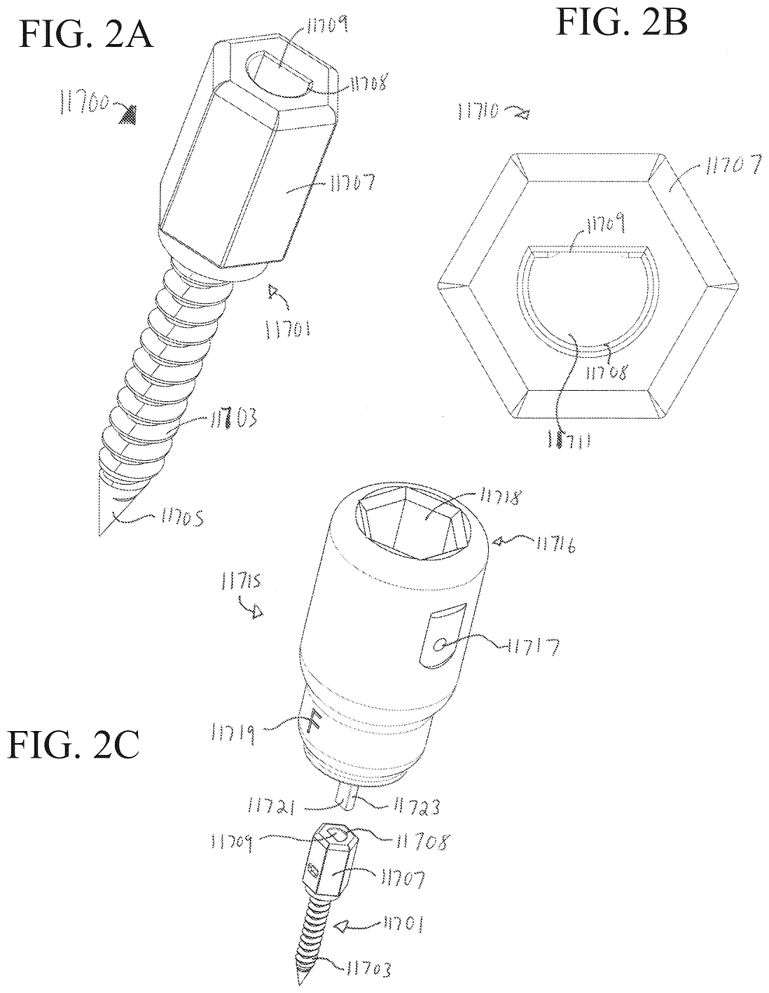

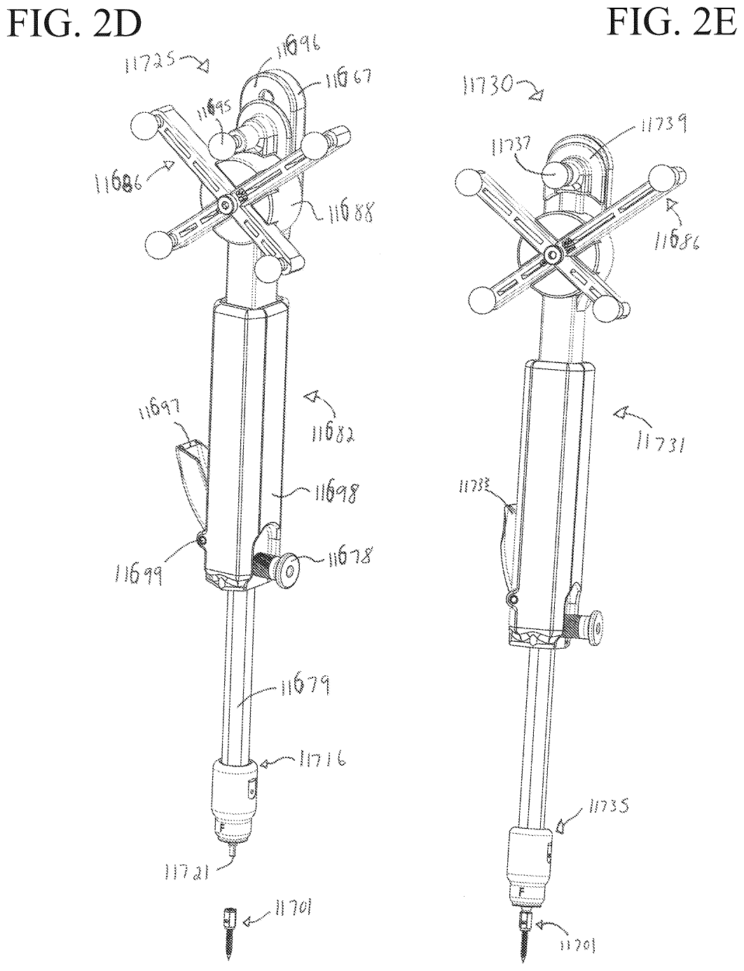

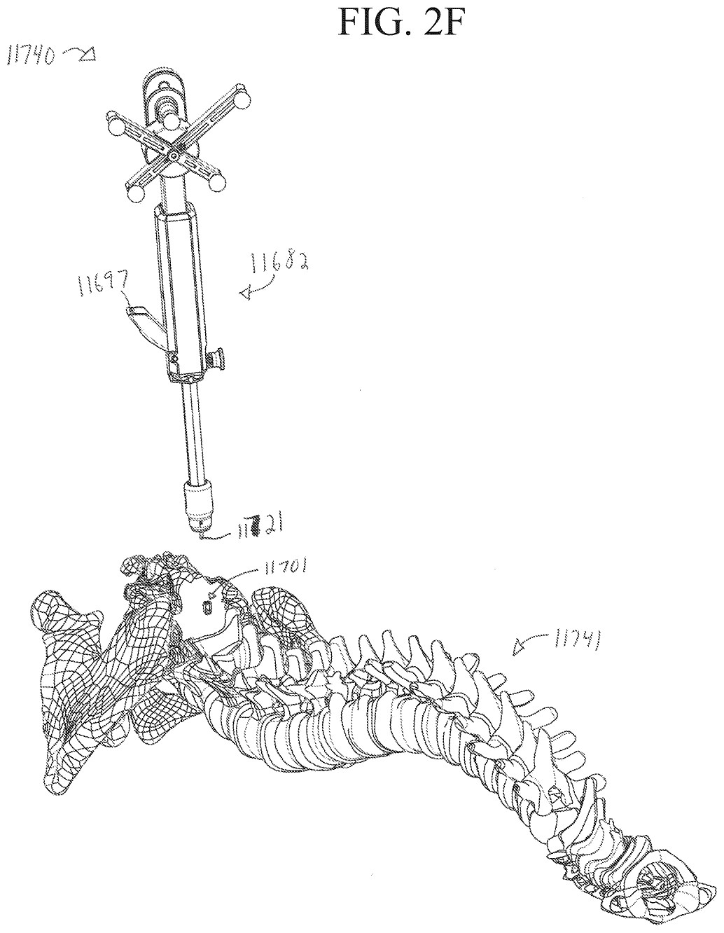

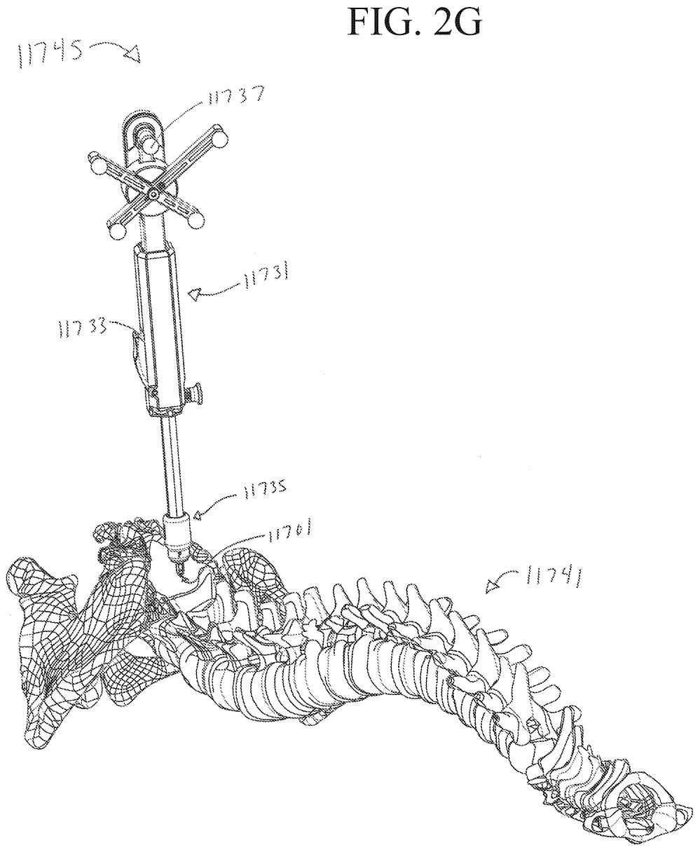

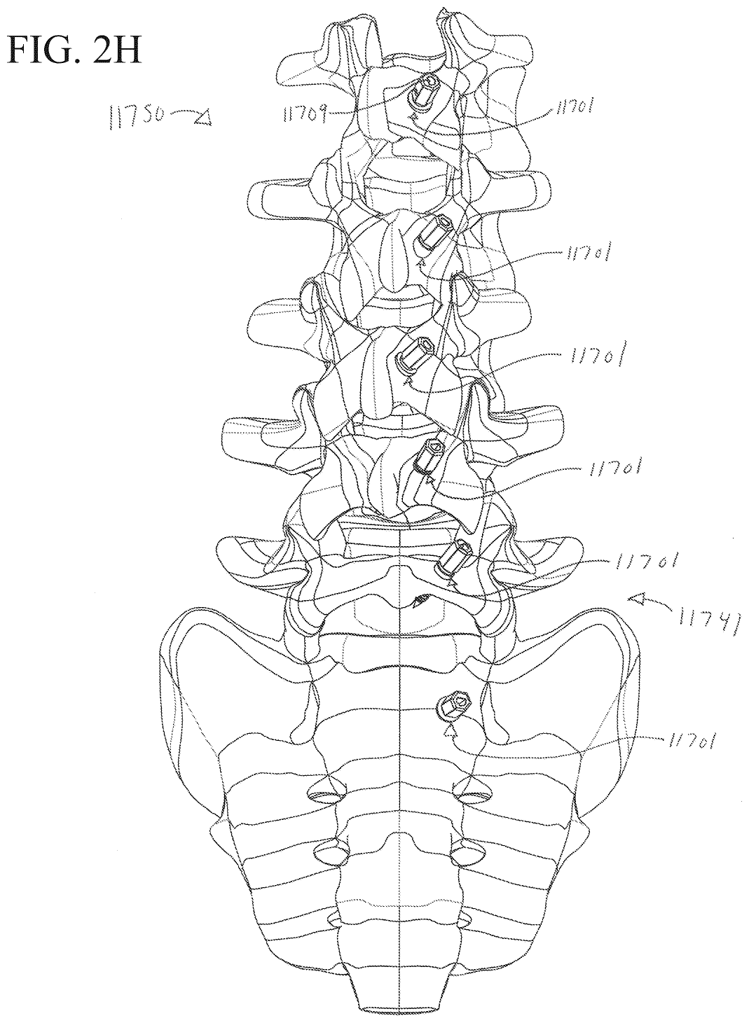

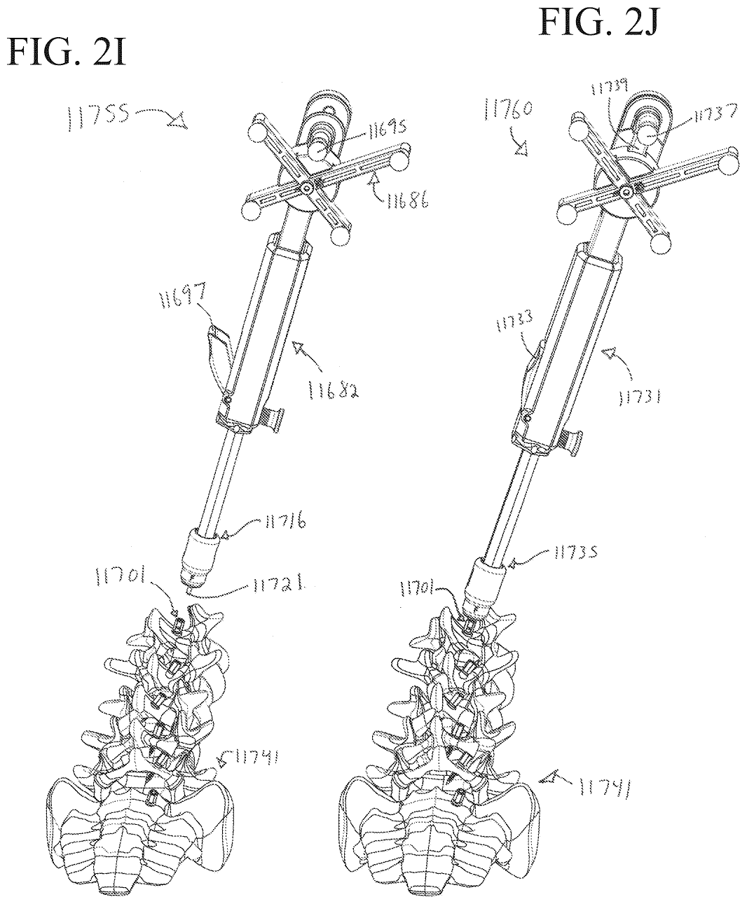

Some embodiments of the invention include a fiducial device that is implanted into bony tissue of anatomical landmarks of interest for precise registration of their 3D locations and poses for purposes of calculating spinal alignment parameters, 3D visualization of anatomy, and updating the registration of image-guided navigation systems and their associated reference systems. In some embodiments, the bone-mounted fiducial devices can be placed anywhere within or near the surgical site when registration of key landmarks linked to the fiducial device are desired, and the device is designed to avoid obstructing the routine anatomical structures manipulated or removed during spine surgical surgeries. In some embodiments, FIG. 2A illustrates the bone-mounted fiducial 11701 that contains a female mating mechanism for registration with a 3D-tracked tool that contains a complementary mating interface. In some embodiments, the fiducial device has screw threads 11703 that align with the mating interface and its screwdriver wall interface 11707. In some embodiments, the mating mechanism of the female direct-screw bone-mounted fiducial 11701 involve a female flat mating interface 11709 and a cylindrical mating interface 11708. In some embodiments, the fiducial mating interface can be any cross-sectional, asymmetric pattern that enables a 3D-tracked tool to register the unique 3D orientation and location of the fiducial device. In some embodiments, the flat mating surface 11709 thus represents the true north orientation axis of the device and enable reliable registration of the device's 3D coordinates axes via probe-based acquisitions.

In some embodiments, FIG. 2B illustrates a top view of the bone-mounted fiducial 11701 as described previously in relation to FIG. 2A. In some embodiments, the female floor mating interface 11711 of the fiducial provides a depth-stop interface for the 3D-tracked probe tip to enable repeatable, uniform measurements of the fiducial's location and orientation with probe acquisitions. In some embodiments, FIG. 2C illustrates a disengaged probe tip adapter 11716 that contains a male fiducial flat protrusion mating interface 11721 and a cylindrical mating interface 11723. In some embodiments, given this complementary, asymmetric mating mechanism, a 3D-tracked probe can only mate with the bone-mounted fiducial 11701 in one unique trajectory and orientation.

In some embodiments of the invention, a 3D-tracked probe registers the unique 3D location and orientation of the bone-mounted fiducial device with a probe tip that can features a complementary mating interface. In some embodiments, FIG. 2D illustrates a perspective view of the bone-mounted fiducial as described previously in relation to FIGS. 2A-C. In some embodiments, the 3D-tracked probe 11682 in this view is not engaged with the bone-mounted fiducial and is in an inactive triggering state. In some embodiments, FIG. 2E illustrates that the 3D-tracked probe 11731 is fully mated with the female bone-mounted fiducial 11701 and the trigger button 11733 actuated the TMSM 11737 into an active state, which signals to the computer to record the current 3D location and orientation of the bone-mounted fiducial 11701.

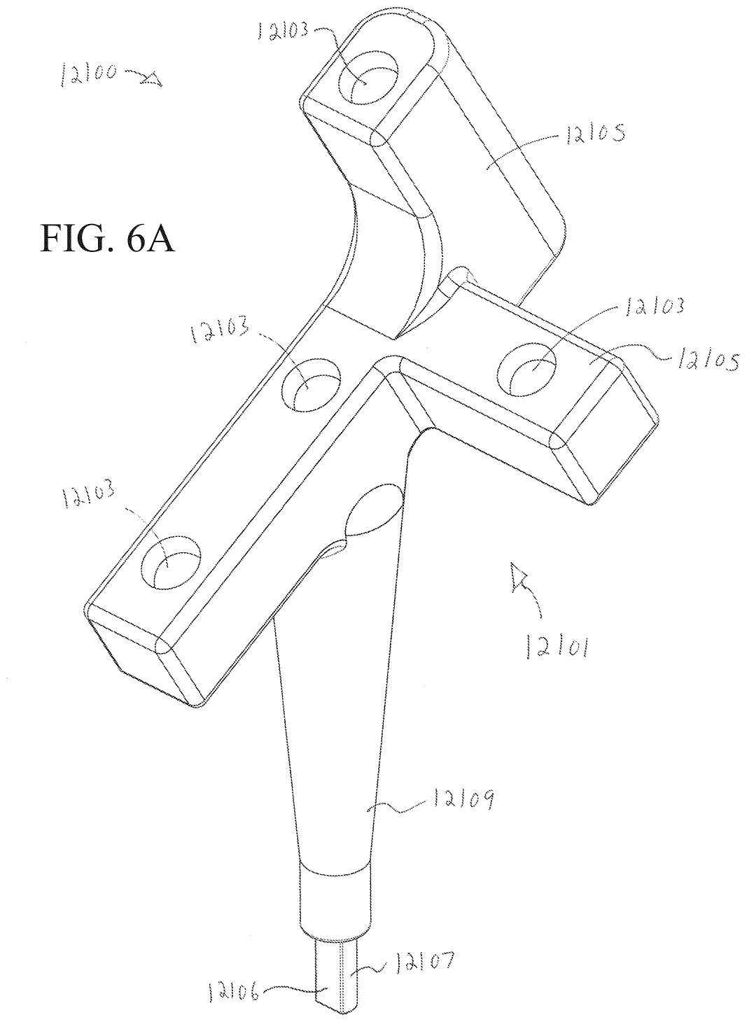

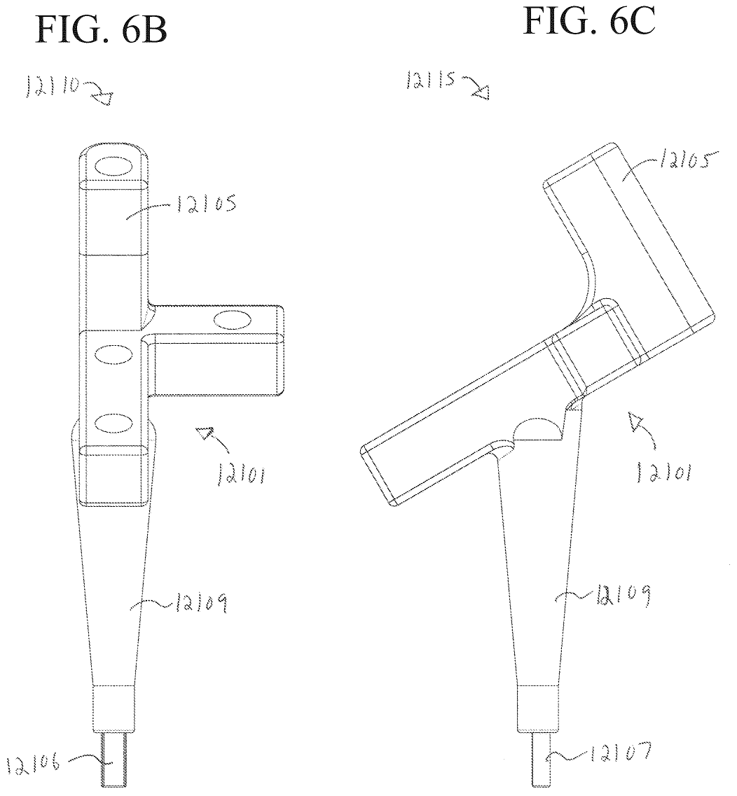

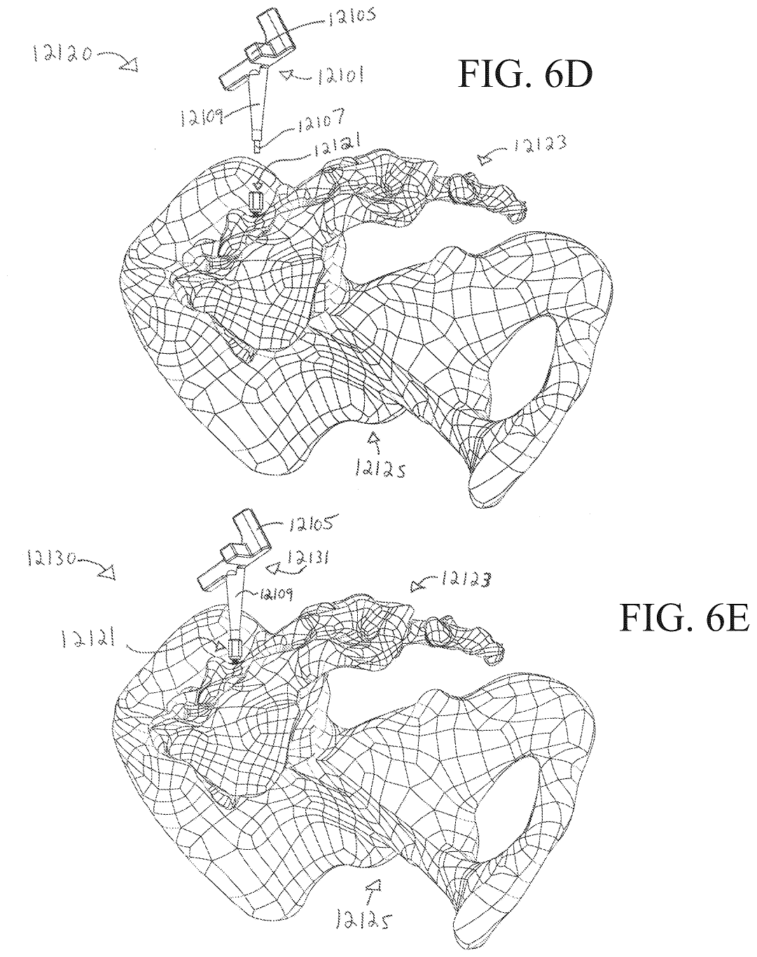

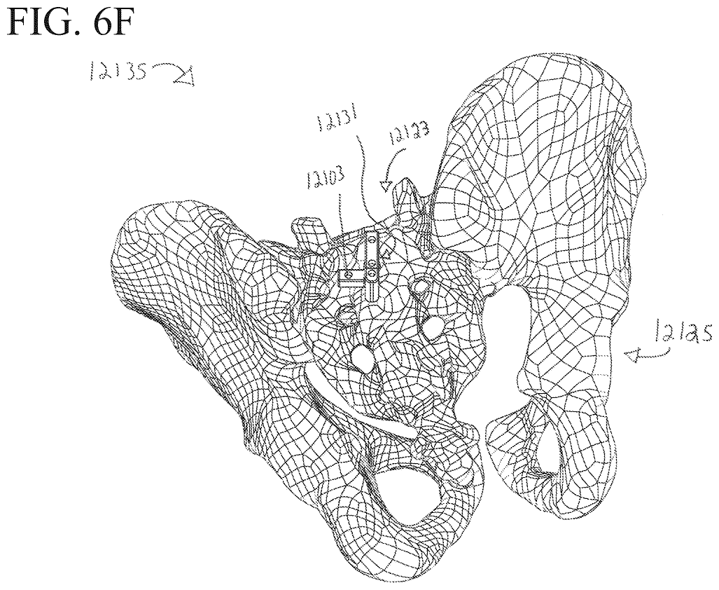

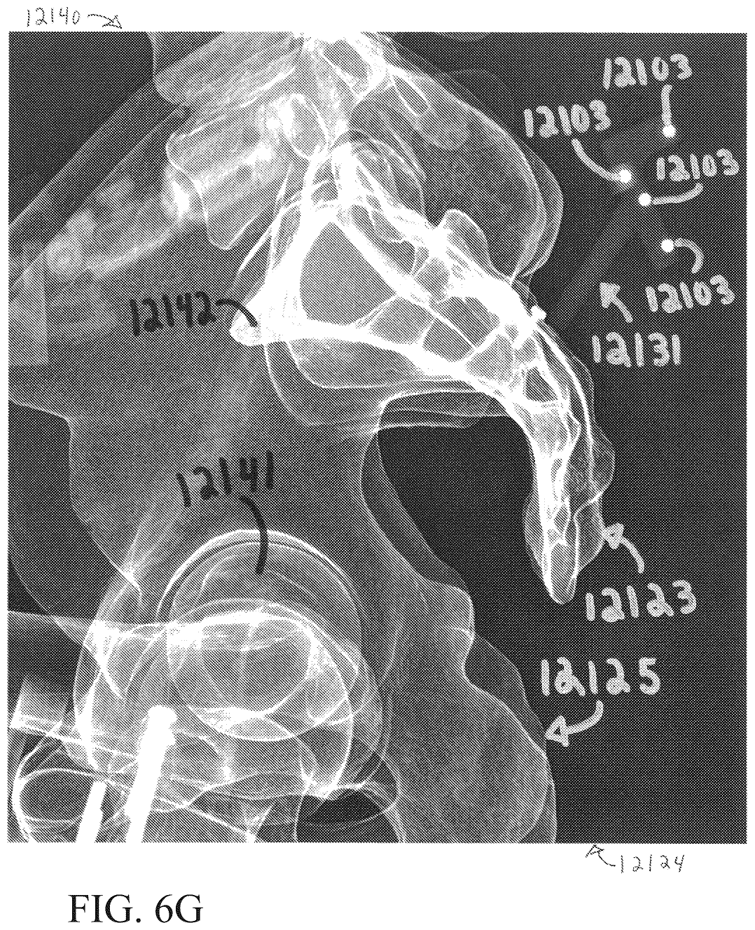

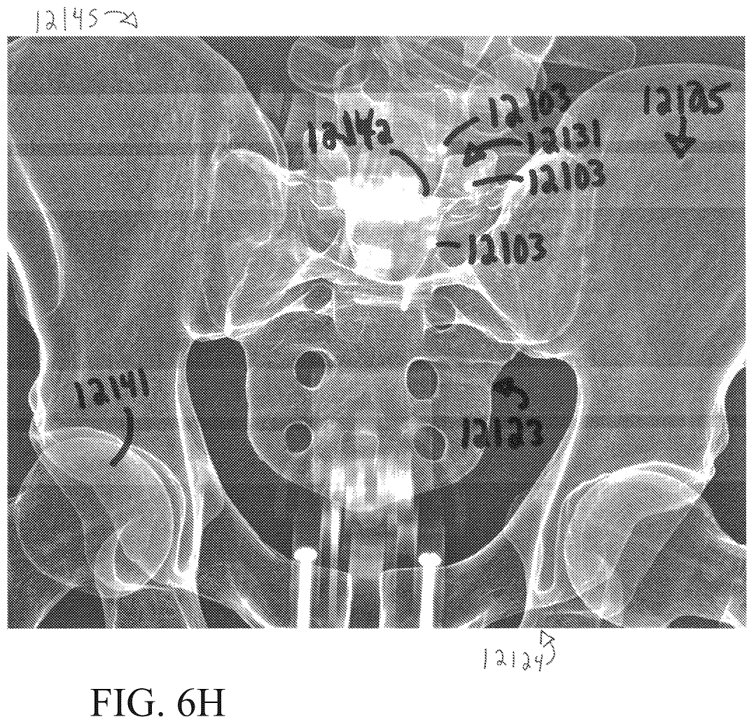

In some embodiments, FIG. 2F illustrates a perspective view of the bone-mounted fiducial 11701, described previously in relation to FIGS. 2A-E, in which the fiducial is implanted into the posterior sacrum of the spine 11741. In some embodiments, the 3D-tracked probe 11682 is not engaged with the bone-mounted fiducial in this view. In some embodiments, FIG. 2G illustrates that the 3D-tracked probe 11731 is fully mated with the implanted bone-mounted fiducial 11701 and the TMSM 11737 is in active state to signal to the computer that the 3D location and pose of the bone-mounted fiducial should now be recorded into the system. In some embodiments, based on prior initialization (e.g., processes shown in FIG. 6 and other enclosed inventions) of the relations of the bone-mounted fiducial device to nearby anatomical landmarks of the interest, the probe-based registration of the bone-mounted fiducial enables for the automated calculation of the up-to-date 3D locations and poses of all bony landmarks of interest relative to the new location and pose of the bone-mounted fiducial.

In some embodiments of the invention, such as FIG. 2H, bone-mounted fiducials 11701 are implanted into multiple vertebrae of the spine to enable the 3D registration of multiple segments of the spine. In some embodiments, after all engaged vertebrae are initialized in relative location and pose to that of each implanted bone-mounted fiducial device 11701, then the probe-based acquisitions of each bone-mounted fiducial provides updated information as to the new location and pose of individual vertebrae. In some embodiments, a useful benefit of this system is simplifying the re-registration of image-guided navigation with the ability to minimize the requirement for additional imaging or complex registration steps that are typically used with current state of the art systems whenever bony landmarks are adjusted relative to their initial registered positions. In some embodiments of the invention, after initialization of each engaged vertebrae, the re-registration of each bone-mounted fiducial can be performed throughout the duration of the surgery with acquisitions via a 3D-tracked tool with a complementary mating interface, without the need for additional imaging or anatomical reference registrations. In some embodiments, FIGS. 2I-2J illustrate the registration process of a bone-mounted fiducial 11701 that is implanted into the spine 11741 which is being rapidly registered via a 3D-tracked probe 11682. In some embodiments, after the 3D-tracked probe 11731 is fully engaged with the bone-mounted fiducial 11701, the trigger button 11733 can actuate the TMSM 11737 and signal to the computer the active registration of a unique bone-mounted fiducial. In some embodiments of the invention, this system of devices and algorithms is used to rapidly update the registration of image-guided surgical navigation systems. In some embodiments, this system rapidly registers the 3D spinal alignment parameters between vertebrae that contain implanted bone-mounted fiducials 11701.

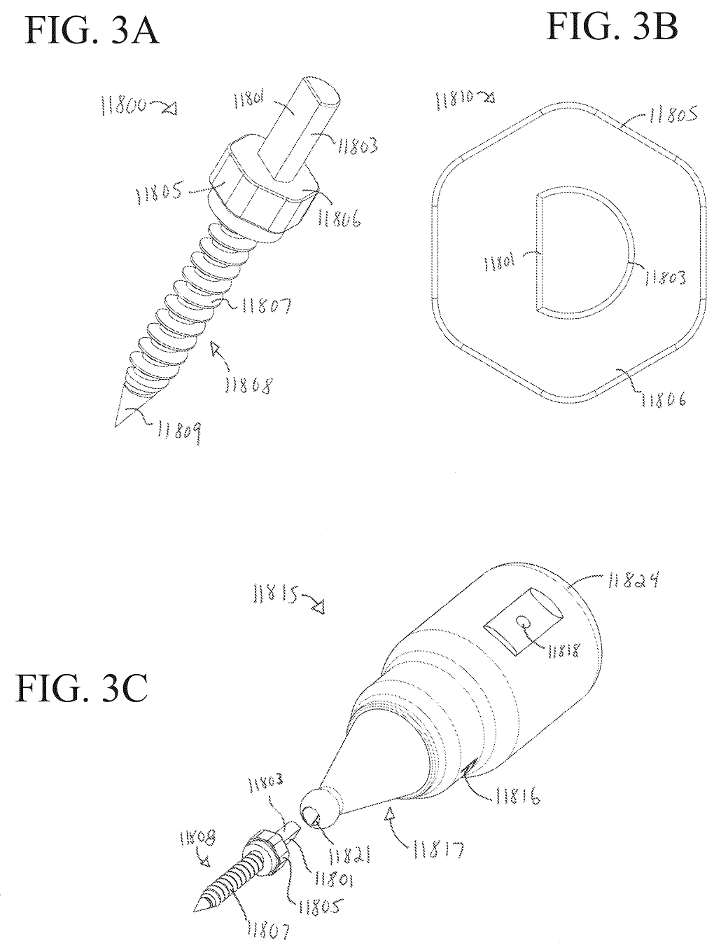

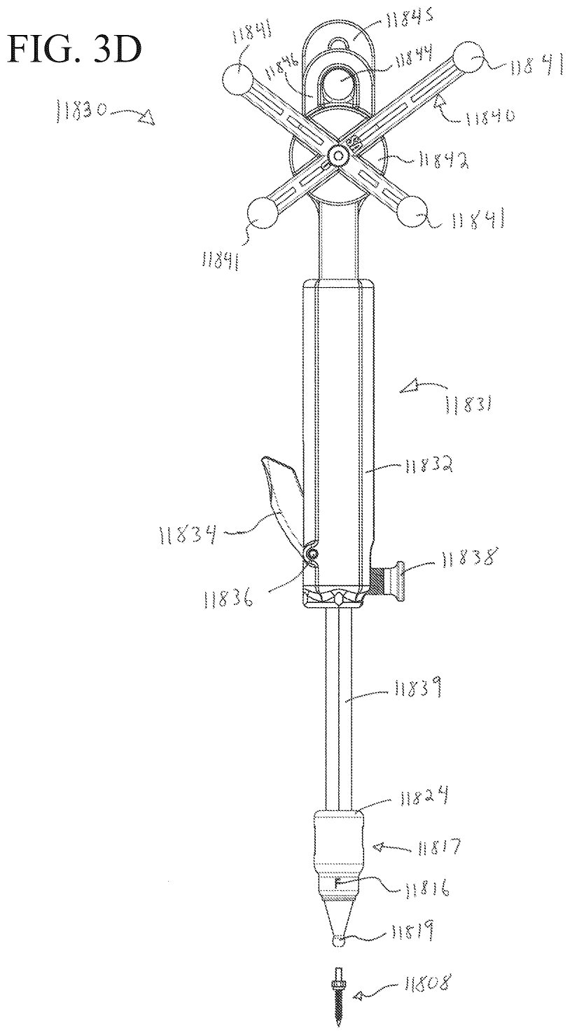

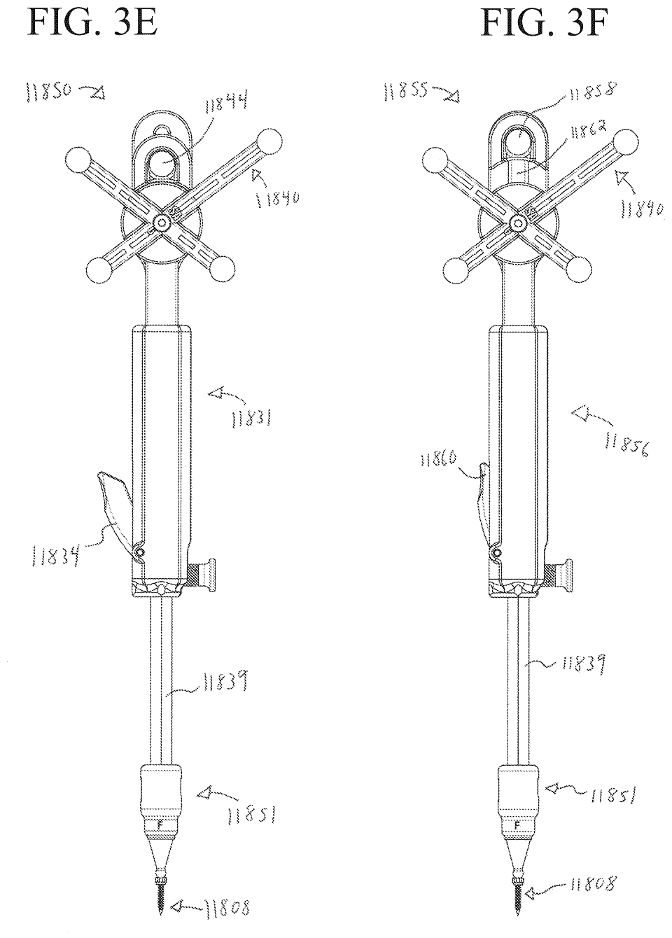

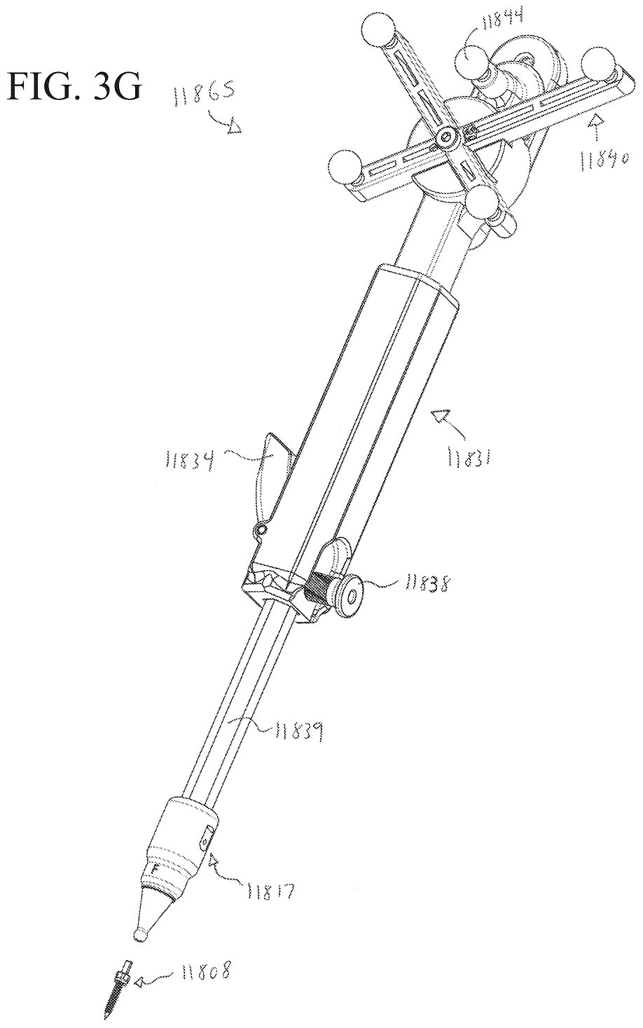

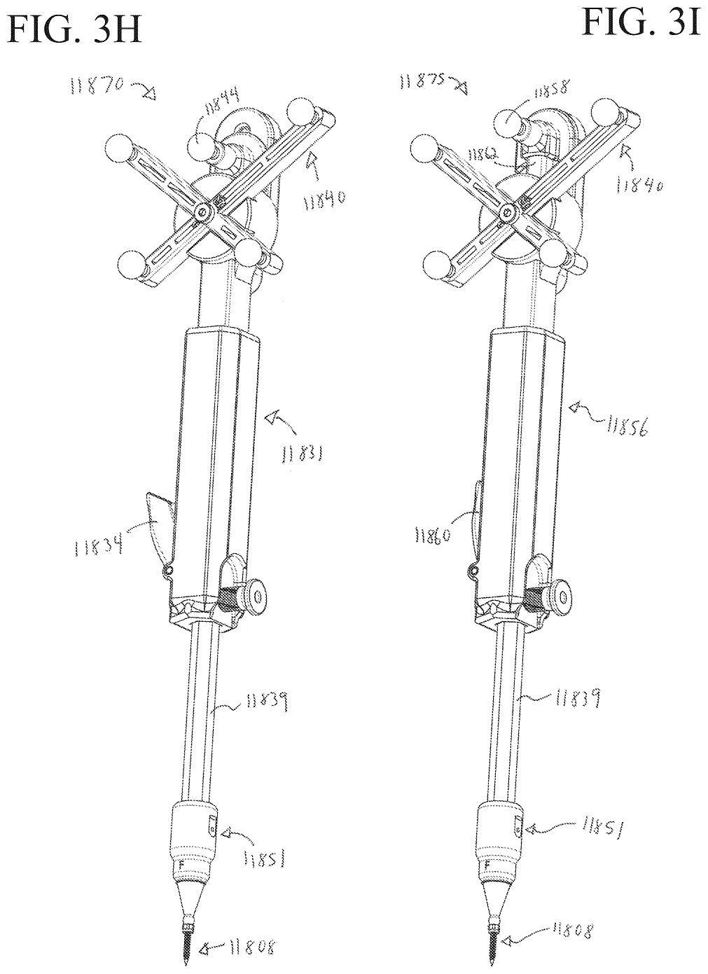

Some embodiments of the invention include a bone-mounted fiducial device with a male mating protrusion that enables precise registration of the fiducial's 3D location and pose for purposes of calculating spinal alignment parameters, 3D visualization of anatomy, and updating the registration of image-guided navigation systems and their associated reference systems. In some embodiments, FIG. 3A illustrates a perspective view of a male bone-mounted fiducial 11808 that is similar in function to previously described devices in relation to FIG. 2. In some embodiments, the fiducial contains screw threads 11808 that are aligned with the long axis of the male protrusion shaft. In some embodiments, the male mating mechanism of the fiducial comprises a male flat mating interface 11801 and a male cylindrical mating interface 11803. In some embodiments, the cross-sectional design of the mating interface can be any pattern that enables unique orientation registration of the fiducial device.

In some embodiments, FIG. 3B illustrates a top view of a bone-mounted fiducial device as described previously in relation to 118A. In some embodiments, the fiducial comprises depth-stop mating interface 11806 that engages directly with a 3D-tracked tool (not shown) to provide a reliable mating interface location and pose to be registered for the fiducial device. In some embodiments, the walls 11811 of the bone-mounted fiducial device are a hexagonal cross-section to enable simple engaging of the fiducial device via a screwdriver for implantation of the fiducial into a vertebra or bony structure of interest. In some embodiments of the invention, the bone-mounted fiducial device is implanted into any rigid bony structure of the human body, including the pelvis, sacrum, femur and other lower extremities, shoulders and other upper extremities, the skull, etc. for precise 3D registration and alignment measurements of engaged bony structures.

In some embodiments, FIG. 3C illustrates a perspective view of a bone-mounted fiducial 11808, as described previously in relation to FIGS. 3A-B, in the process of mating with a 3D-tracked probe tip 11815 (shaft and upper device not shown), in which the probe tip contains a female flat mating surface 11821 and a female cylindrical mating surface 11823. In some embodiments of the invention, a 3D-tracked probe 11831 is used to register the 3D location and orientation of a bone-mounted fiducial device 11808. In some embodiments, FIGS. 3E-3F illustrate the process of engaging a bone-mounted fiducial device 11808 with a 3D-tracked probe 11831 and then depressing the trigger button 11860 to actuate the TMSM 11858 into an active-state location with respect to the fixed position of the 3D-tracked DRF 11840. In some embodiments, FIGS. 3G-31 illustrate perspective views of the process of engaging a 3D-tracked probe 11831 with a probe tip adapter 11817 that contains a spherical tip and internal female mating mechanism that engages with the bone-mounted fiducial and then registers the current 3D location and pose of the bone-mounted fiducial device 11808. In some embodiments, once the 3D-tracked probe 11831 is rigidly, uniquely engaged with the bone-mounted fiducial 11808, the TMSM 11844 is actuated via depression of the probe's trigger button 11834 and then the 3D-tracked probe 11856 is actively registering the location and orientation of the bone-mounted fiducial for at least one camera frame in which the TMSM 11858 remains in an active-state location with respect to the probe's DRF 11840.

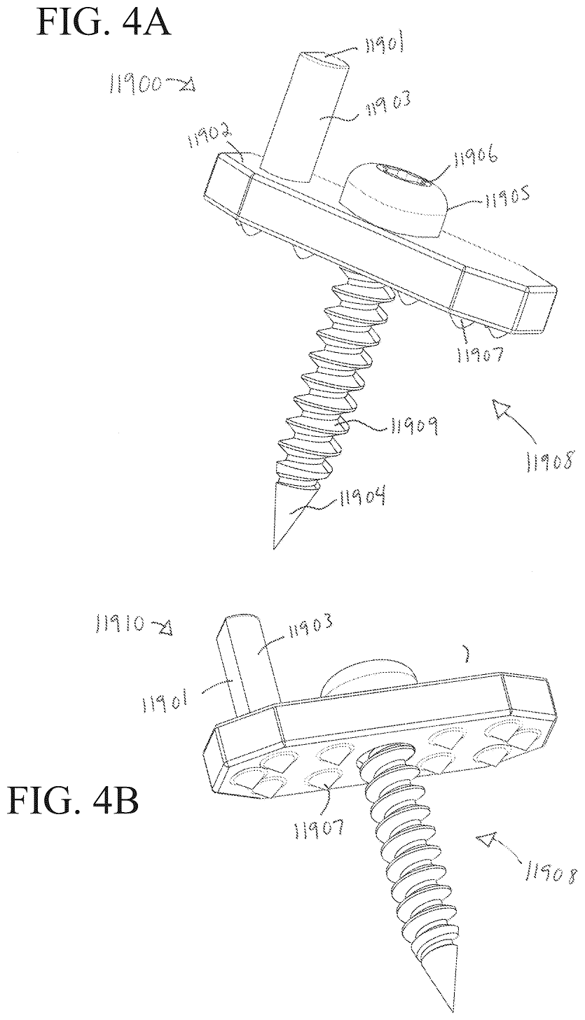

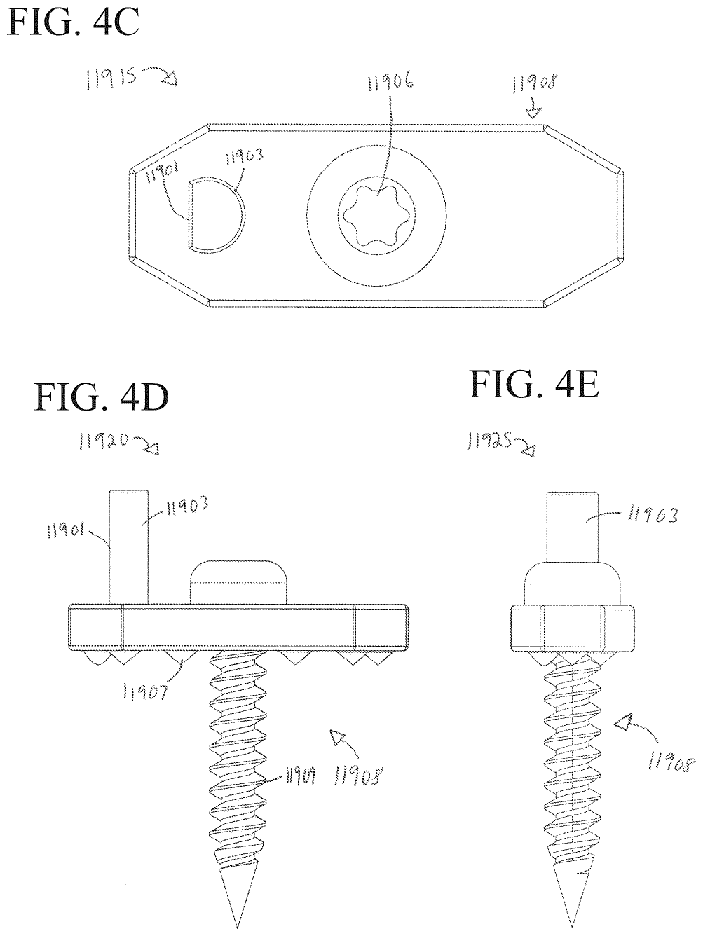

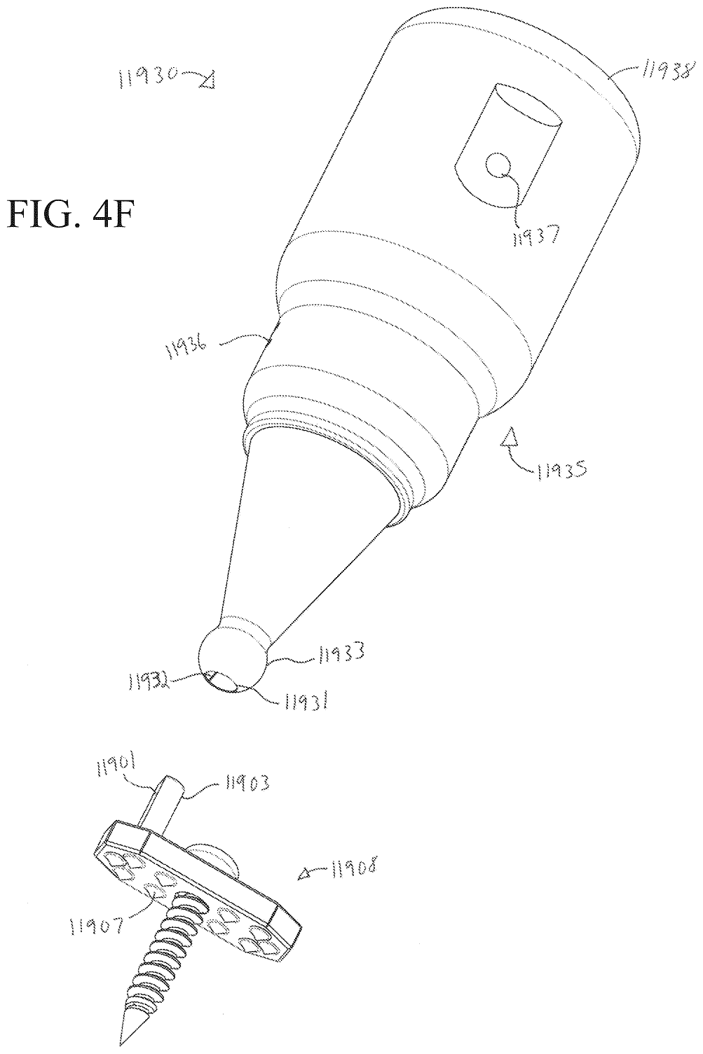

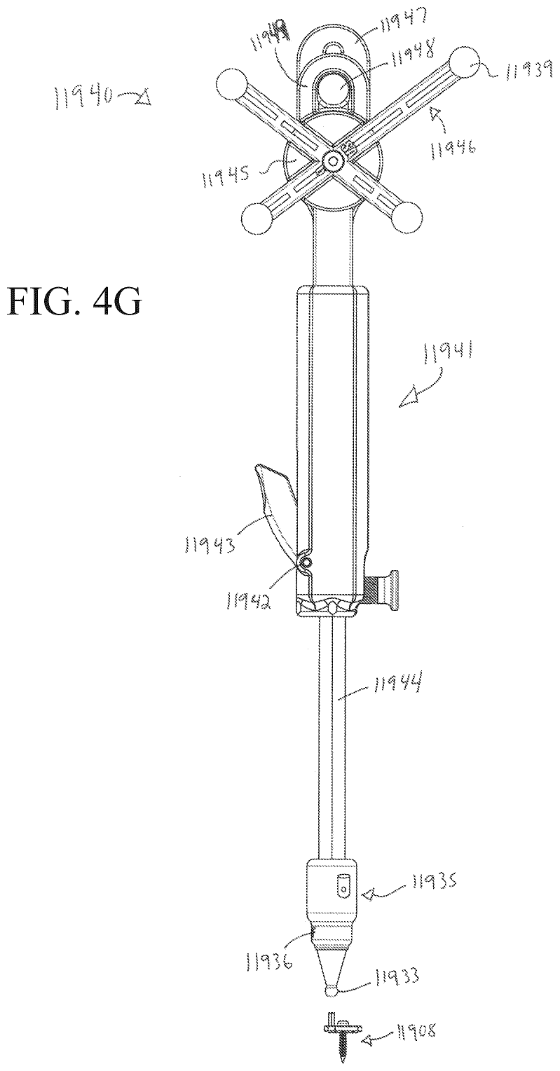

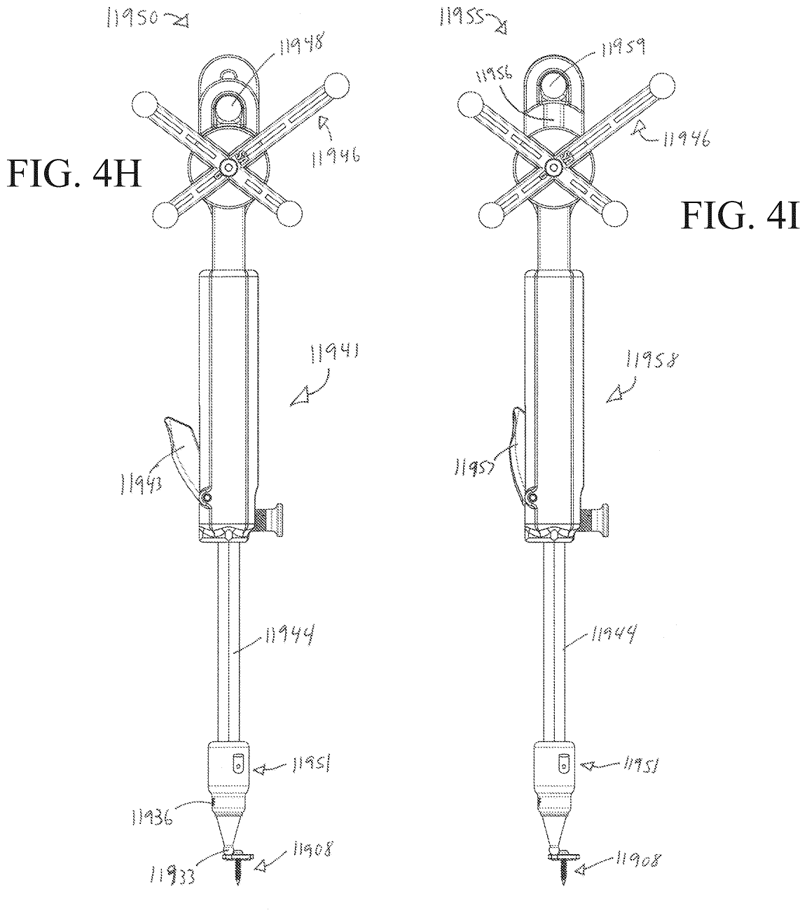

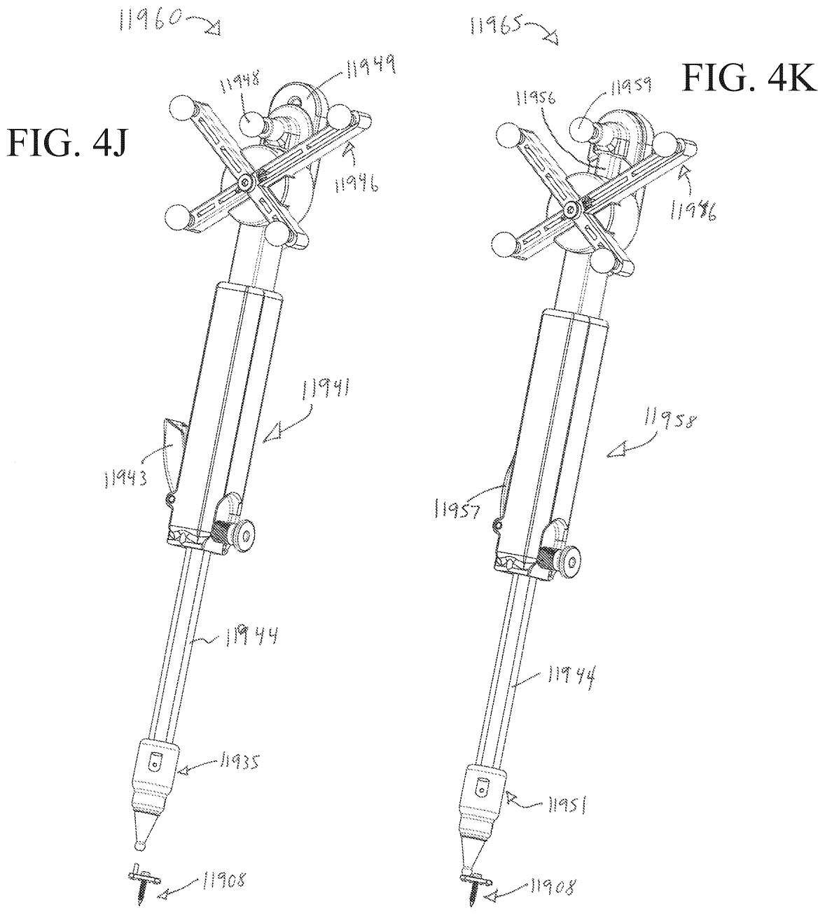

Some embodiments of the invention include a bone-mounted fiducial device with a male mating interface that, when registered, enables precise registration of the fiducial's 3D location and pose for purposes of calculating spinal alignment parameters, 3D visualization of anatomy, and updating the registration of image-guided navigation systems and their associated reference systems. In some embodiments, FIG. 4A illustrates a perspective view of the bone-mounting fiducial device, which is similar in function to previously described devices in relation to FIGS. 2-3, shown with a male mating protrusion offset from the device screw shaft axis. In some embodiments, the bone-mounted fiducial 11908 comprises a male mating component that contains a flat mating interface 11901 and a cylindrical mating interface 11903. In some embodiments, the device screw threads 11909 are offset from the axis of the mating interface's shaft to enable simplified implantation of the bone-mounted fiducial in a particular orientation on the attached anatomy that enables the direct visualization of a 3D-tracked tool registering the fiducial device 11908 via a 3D-tracking camera. In some embodiments, the flat mating interface 11901 determines the north orientation of the fiducial device 11908 and thus needs to be oriented towards the direction of the 3D-tracking camera. In some embodiments, FIG. 4B illustrates a perspective view of the underside of the bone-mounted fiducial device 11908, which in some embodiments contains a series of frictional surface spikes 11907 on the bottom surface of the depth-stop mating interface 11902.

In some embodiments, FIG. 4C illustrates a top view of a bone-mounted fiducial device 11908 as described previously in relation to FIGS. 4A-B. In some embodiments, the bone-mounting screw contains a torx head driver interface 11906. In some embodiments, FIGS. 4D-4E illustrate side views of the bone-mounted fiducial device 11908.

In some embodiments, FIG. 4F illustrates a perspective view of a bone-mounted fiducial device 11908, as described previously in relation to FIGS. 4A-C, in the process of mating with a 3D-tracked probe tip 11935 (probe not shown) that contains a complementary, female mating interface that contains a flat surface 11932 and a cylindrical surface 11931.

In some embodiments, FIGS. 4G-I illustrates a front view of a bone-mounted fiducial device 11908, as described previously in relation to FIGS. 4A-D, and the complete process of mating with a 3D-tracked probe 11941, which contains a complementary, female mating interface. In some embodiments, the bone-mounted fiducial device's 3D location and orientation are registered via a 3D-tracked probe with a complementary mating interface and a trigger button 11943 that actuates a TMSM 11948 beyond a defined 3D location threshold relative to the fixed position of a 3D-tracked DRF 11946. In some embodiments, when the trigger button 11957 is fully depressed, the TMSM 11959 surpasses a defined location threshold that the system interprets as the 3D-tracked probe 11958 being in an active, registration state. In some embodiments, FIGS. 4J-4K illustrate perspective views of the bone-mounted fiducial device 11908 and a 3D-tracked probe 11941 in the process of mating as described previously in relation to FIGS. 4A-4I.

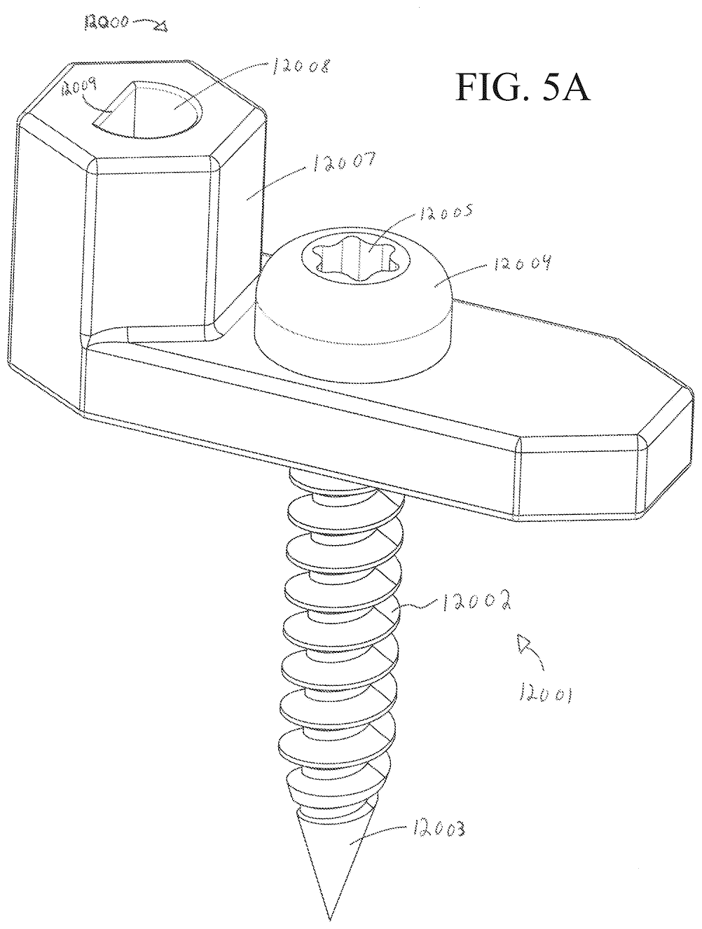

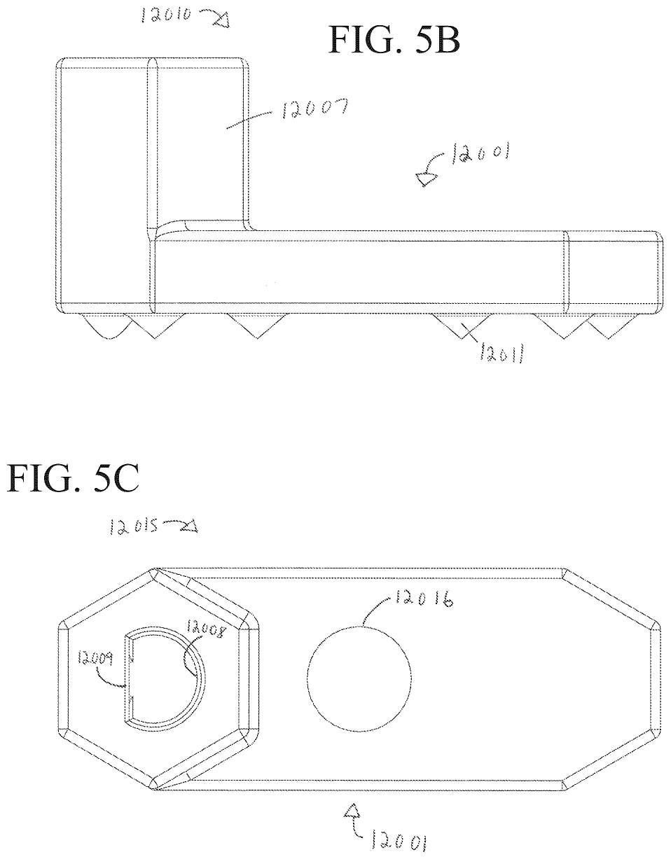

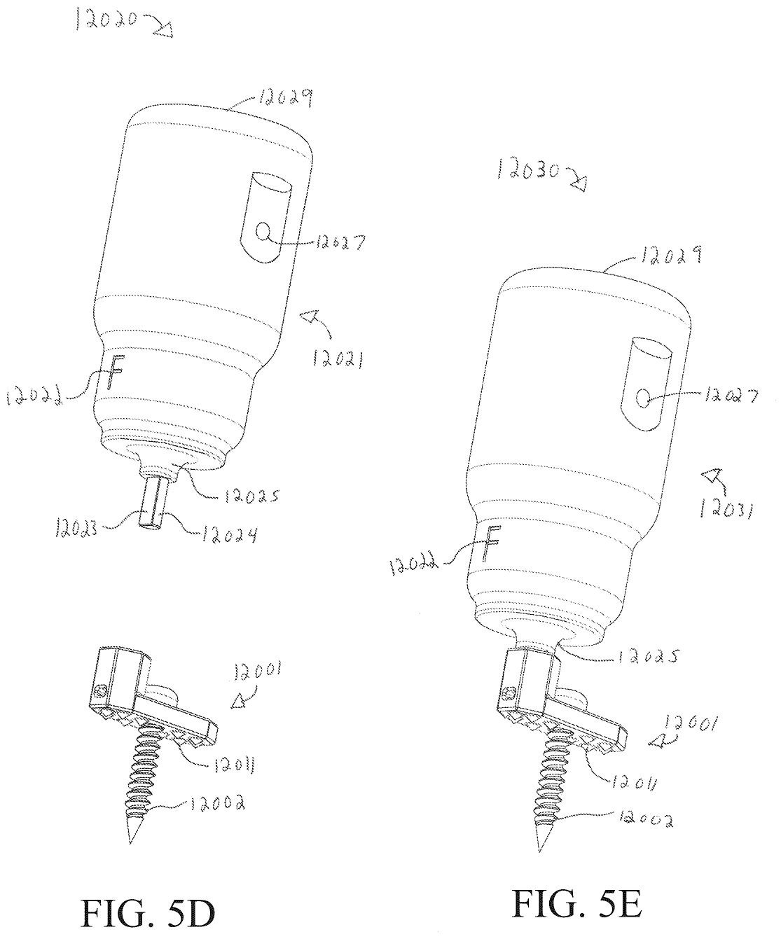

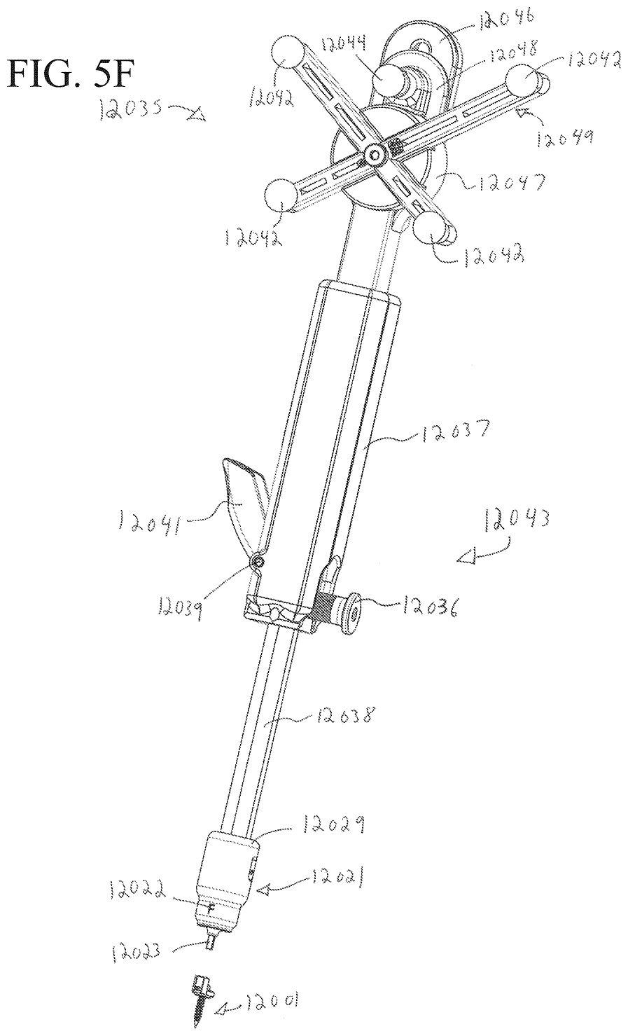

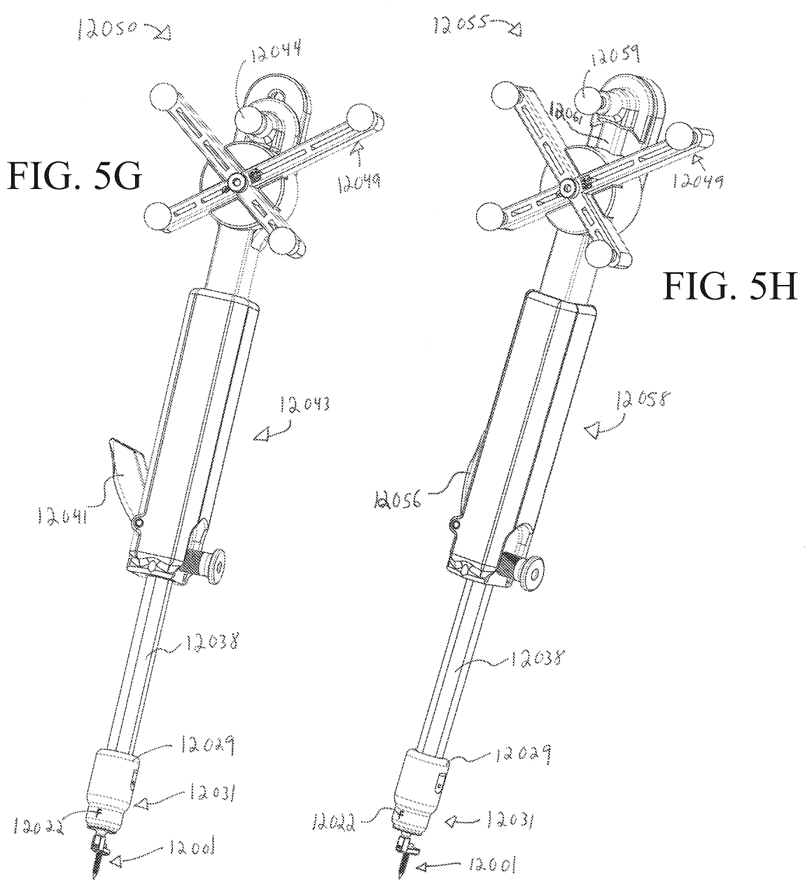

Some embodiments of the invention include a bone-mounted fiducial device with a female mating interface that, when registered, enables precise registration of the fiducial's 3D location and pose for purposes of calculating spinal alignment parameters, 3D visualization of anatomy, and updating the registration of image-guided navigation systems and their associated reference systems. In some embodiments, FIG. 5A illustrates a perspective view of a bone-mounted fiducial device 12001, which is similar in function to previously described devices in relation to FIGS. 2-4. In some embodiments, the bone-mounted fiducial device 12001 includes a female mating mechanism with a flat mating surface 12009 and a cylindrical mating surface 12008 that is offset from the screw shaft axis. In some embodiments, the screw threads 12002 come to a tapered tip 12003 in some embodiments. In some embodiments, the screw head 12004 is a torx head driver interface 12005.

In some embodiments, FIG. 5B illustrates a side view of the bone-mounted fiducial device 12001 that is previously described in relation to FIG. 5A. In some embodiments, the device is equipped with friction spikes 12011 on the bottom surface to enable rigid fixation to engaged bony structures. In some embodiments, FIG. 5C illustrates a top view of the fiducial device 12001. In some embodiments, the female flat mating surface 12009 indicates the north orientation of the device when registered via a 3D-tracked tool (not shown) that contains a complementary mating surface.

In some embodiments, FIGS. 5D-5E illustrate a perspective view of a 3D-tracked male mating probe tip 12021 (probe not shown) in the process of engaging in a unique trajectory with the bone-mounted female device 12001. In some embodiments, when the male mating probe tip 12031 is fully engaged with the female bone-mounted fiducial device 12001, the 3D-tracked probe (not shown) can acquire the 3D location and pose of the fiducial device. In some embodiments, FIGS. 5F-5H illustrate a perspective view of a 3D-tracked probe 12043 and the female mating bone-mounted fiducial device 12001 in the process of mating together and the 3D-tracked probe 12043 acquiring the 3D location and orientation of the fiducial device 12001. In some embodiments, FIG. 5H illustrates the 3D-tracked probe 12058 with a male mating probe tip 12023 fully engaged into the female mating bone-mounted fiducial device 12001, and as the trigger button 12056 is fully depressed, the TMSM 12059 actuated via the TMSM backing mount 12061 is in an active-state location with respect to the fixed location of the DRF 12049.

Some embodiments of the invention include an X-Ray adapter device that mates in a unique orientation with a bone-mounted fiducial device. In some embodiments, the X-Ray adapter device contains radiopaque sphere fiducials used for initialization of the location and pose of nearby anatomical landmarks of interest relative to the implanted bone-mounted fiducial device. In some embodiments of the invention, the X-Ray adapter device is used to initialize the 3D location and pose of key anatomical landmarks such as the S1 endplate, femoral heads, L5, L1, T10, T9, T4, T1, C1, C7, occiput, etc. to calculate spinal alignment measurements. In some embodiments, FIG. 6A illustrates a perspective of an example embodiment of the X-Ray adapter device 12101 that contains three or more radiopaque fiducial spheres 12103 within the fiducial body 12105, and a male mating interface comprising a male flat surface 12106 and male cylindrical surface 12107 that engages with a female mating interface of a fiducial device (not shown) in a unique orientation. In some embodiments, the arrangement of radiopaque fiducial spheres 12103 involves an asymmetric distribution of spheres in a 3D-offset pattern that produces a unique 3D pose for all rotational views of the sphere arrangement. In some embodiments, the system can use the projection of inter-sphere relations in an X-Ray image to automatically detect the 3D pose of the fiducial with respect to the X-Ray detector imaging plane. In some embodiments, FIGS. 6B-6C illustrate a side view of the X-Ray adapter device 12101 and highlight the unique orientation of the flat 12106 and cylindrical 12107 mating surfaces.

In some embodiments, FIGS. 6D-6E illustrates a side view of the process of mating the male X-Ray adapter device 12101 into an implanted female bone-mounted fiducial device 12121, as described previously in relation to FIGS. 2A-2J. In some embodiments, the X-Ray adapter device 12131 engages into an implanted bone-mounted fiducial device 12121 that is engaged with the sacrum 12123 and the pelvis 12125 of the spine. In some embodiments, two or more multi-planar X-Ray images are acquired of the X-Ray adapter device 12101 while engaged into a bony landmark of interest for 3D registration of the anatomical landmark's 3D location and unique pose. In some embodiments, FIG. 6F illustrates a top view of the X-Ray adapter device 12131 engaged in the sacrum 12123, with the engaged adapter device containing four radiopaque fiducial spheres arranged in a unique, asymmetric geometric distribution to facilitate auto-pose detection of the fiducial in X-Ray images relative to the detector imaging plane.

In some embodiments, FIG. 6D illustrates a lateral X-Ray image 12124a of the X-Ray adapter device 12131 engaged into an implanted bone-mounted fiducial device. In some embodiments, the radiopaque fiducial spheres can be visualized in their unique pose relative to the imaging detector plane. In some embodiments, the user annotates key anatomical landmarks of interest and the system calculates their respective locations relative to the calculated centroid of the four, radiopaque sphere 12103 coordinates. In some embodiments, the S1 endplate 12142, femoral heads 12141, and other landmarks of the sacrum 12123 and pelvis 12125 are annotated and automatically registered by the computer in relation to the fiducial device. In some embodiments, FIG. 6H illustrates an AP X-Ray image 12124b of the X-Ray adapter device 12131 engaged into an implanted bone-mounted fiducial device. In some embodiments, the detected 3D-pose of the fiducial adapter device 12131 relative to the imaging detector plane is converted in a rigid body transform and then applied to all annotated anatomical landmarks of interest to compute the 3D vectors of each anatomical landmark relative to the fiducial adapter device's 12131 coordinate axes.

In some embodiments, the mating interface of the adapter device 12101 comprises a quarter-turn twist mating mechanism to ensure reliable, complete mating of the adapter device with the bone-mounted fiducial device. In some embodiments, the inter-sphere angles computed between the 2D-projected coordinates of radiopaque fiducial spheres 12103 are automatically analyzed by the computer to identify the anatomical axes of the patient relative to the X-Ray images (e.g., inferior end, posterior end, and right end of patient relative to image).

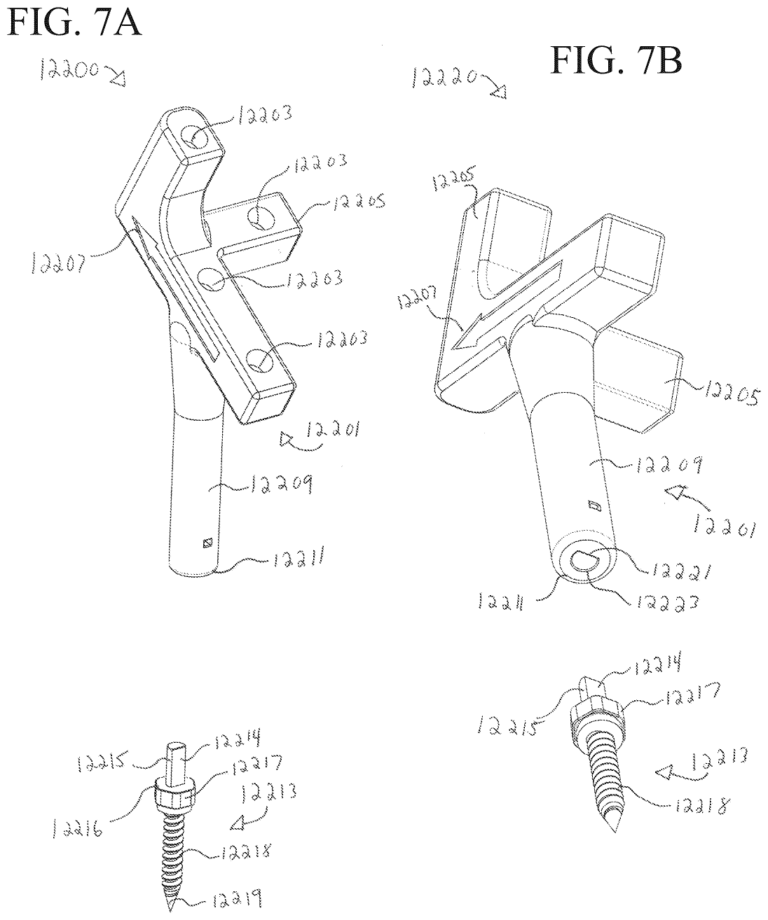

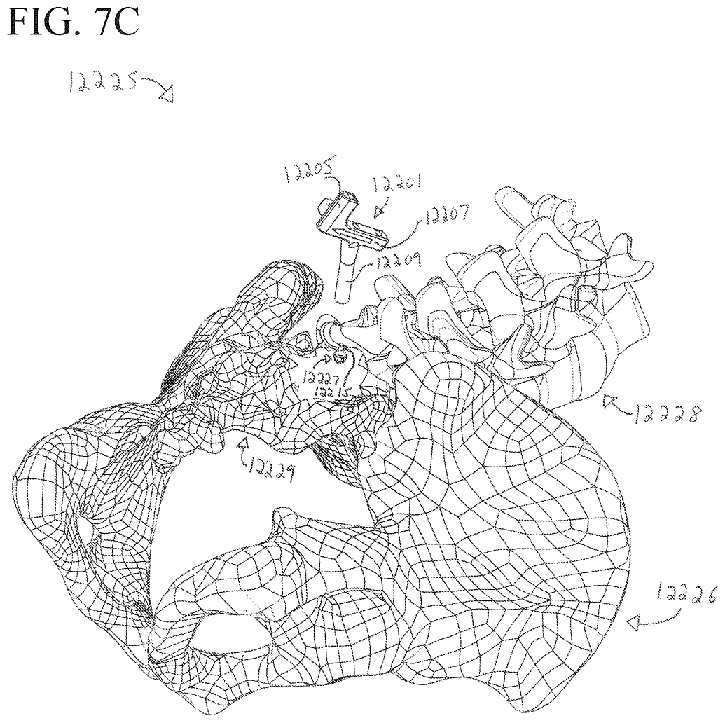

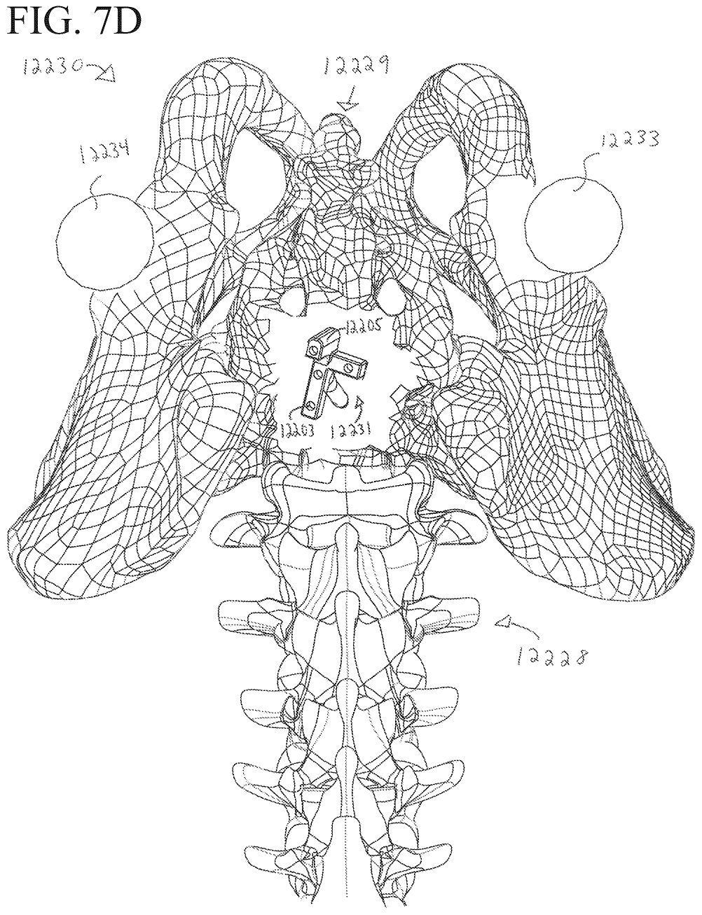

In some embodiments of the invention, the X-Ray adapter device includes an orientation indicator to aid in defining patient anatomical axes and orientation relative to the X-Ray images of the adapter device. In some embodiments, FIGS. 7A-7B illustrate a perspective view of a female-mating X-Ray adapter device 12201, which is described previously in relation to FIGS. 6A-6H, that mates in a unique 3D trajectory and orientation with a bone-mounted, male-mating fiducial device 12213, as previously described in relation to FIGS. 3A-31. In some embodiments, the orientation indicator 12208 is used to aid the user to indicate in the X-Ray image which direction the arrow of the fiducial orientation indicator 12208 is pointed with relation to nearby anatomical axes. In some embodiments, the female mating interface of the X-Ray adapter device comprises a flat edge surface 12221 and a cylindrical surface 12223 that matches the cross-sectional male interface pattern of the bone-mounted fiducial device 12213. In some embodiments, FIGS. 7C-7D illustrate the mating process of the X-Ray adapter device 12201 to the implanted bone-mounted male-mating fiducial device 12225 engaged in the sacrum 12229. In some embodiments, the X-Ray adapter device is fully engaged with the bone-mounted fiducial device when the adapter's shaft mating interface 12211 engages with the top surface of the bone-mounted fiducial's depth-stop mating interface 12216. In some embodiments, FIG. 7D illustrates an example embodiment of the X-Ray adapter device 12231 from a top view as the system is ready for X-Ray images for initialization of anatomical landmarks of the spine 12228, such as the femoral heads 12234, and other engaged landmarks of the sacrum 12228 or pelvis 12226.

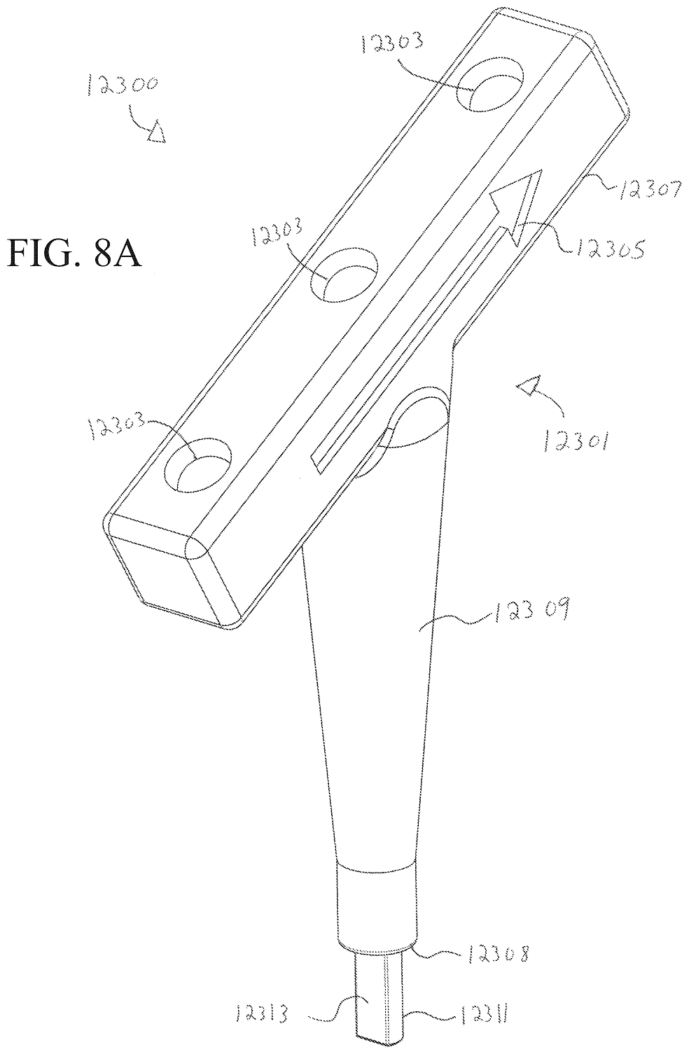

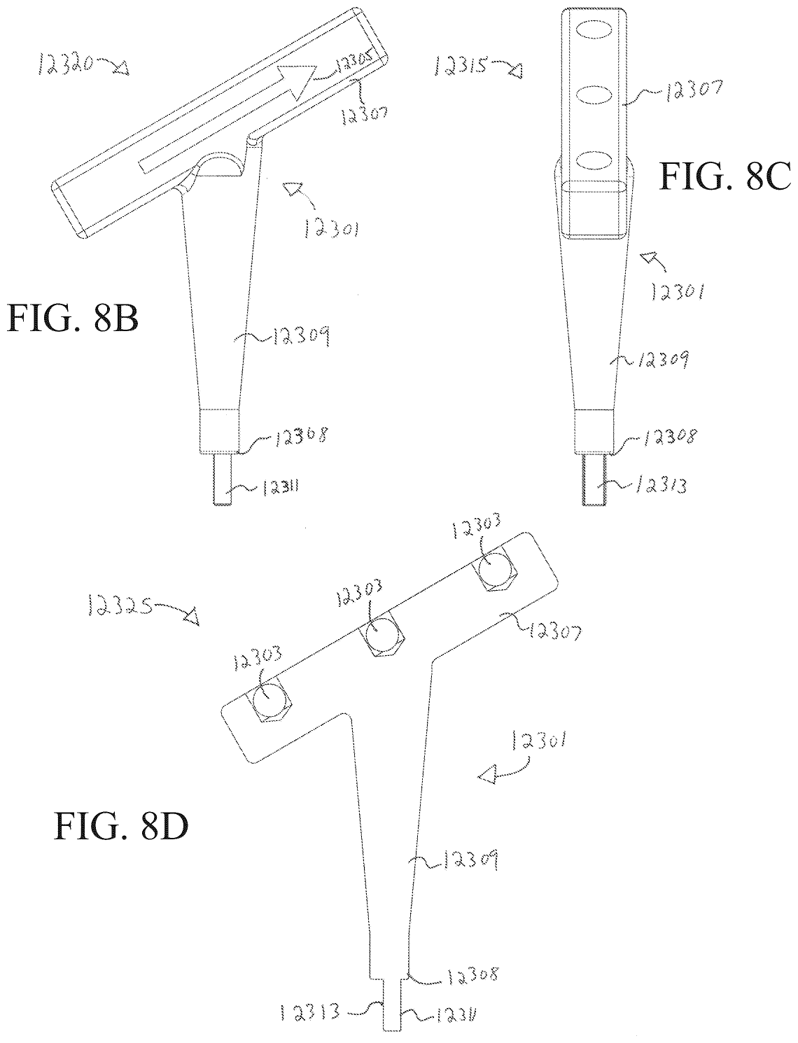

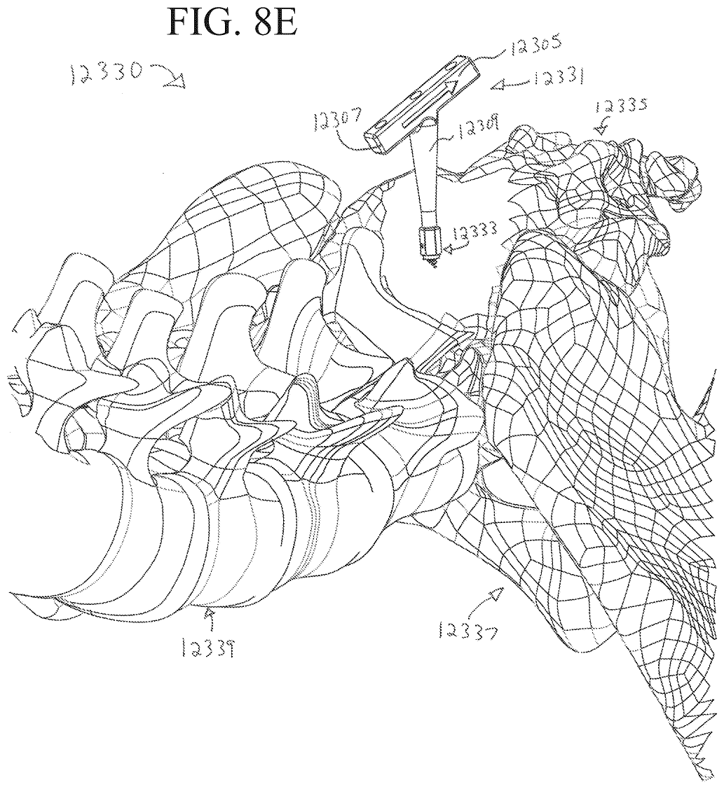

In some embodiments of the invention, the X-Ray adapter device, which is similar in function to the previously described embodiments in relation to FIGS. 6A-6H, 122A-7D, contains a linear or co-planar arrangement of embedded, radiopaque fiducial spheres and an associated orientation indicator sign for the user. In some embodiments, FIG. 8A illustrates a perspective view of the example embodiments of an X-Ray adapter device 12301 with a linear arrangement of radiopaque fiducial spheres 12303 that are parallel in orientation to that of the orientation indicator 12305 of the device. In some embodiments, the X-Ray adapter device 12301 contains a male mating mechanism comprising a flat edge surface 12313 and a cylindrical surface 12311 that fully engages with a bone-mounted fiducial device (not shown) when the adapter-to-fiducial, depth-stop mating interface 12308 is engaged with a fiducial device's depth stop.

In some embodiments, FIGS. 8B-8D illustrate side and cross-sectional views of the X-Ray adapter device as previously described in relation to FIG. 8A. In some embodiments, the sloped angle of the fiducial body 12307 can be adjusted to better accommodate the normative anatomical geometric dimensions of the spine. In some embodiments, the fiducial body 12307 is sloped to accommodate the adapter device being able to mate with a fiducial device on the sacrum, or other steeply-sloped anatomical landmarks, without colliding with nearby anatomical structures and to ensure that the primary plane connecting the radiopaque fiducial spheres 12303 are parallel to the imaging detector plane for maximal accuracy of 3D pose detection. In some embodiments, the X-Ray adapter device 12331 engages in a unique trajectory and orientation with a female-mating bone-mounted fiducial device 12333 implanted into a bony landmark of interest in the sacrum 12335. In some embodiments, the user indicates anatomical axes directions, via the orientation indicator 12305, to the computer system that is analyzing the X-Ray images of this device with respect to nearby anatomical landmarks.

In some embodiments of the invention, a bone-mounted fiducial device with an internal protrusion enables a system for rapidly registering the 3D location and pose of bone-mounted fiducial devices for purposes of calculating spinal alignment parameters, 3D visualization of anatomy, and updating the registration of image-guided navigation systems and their associated reference systems.

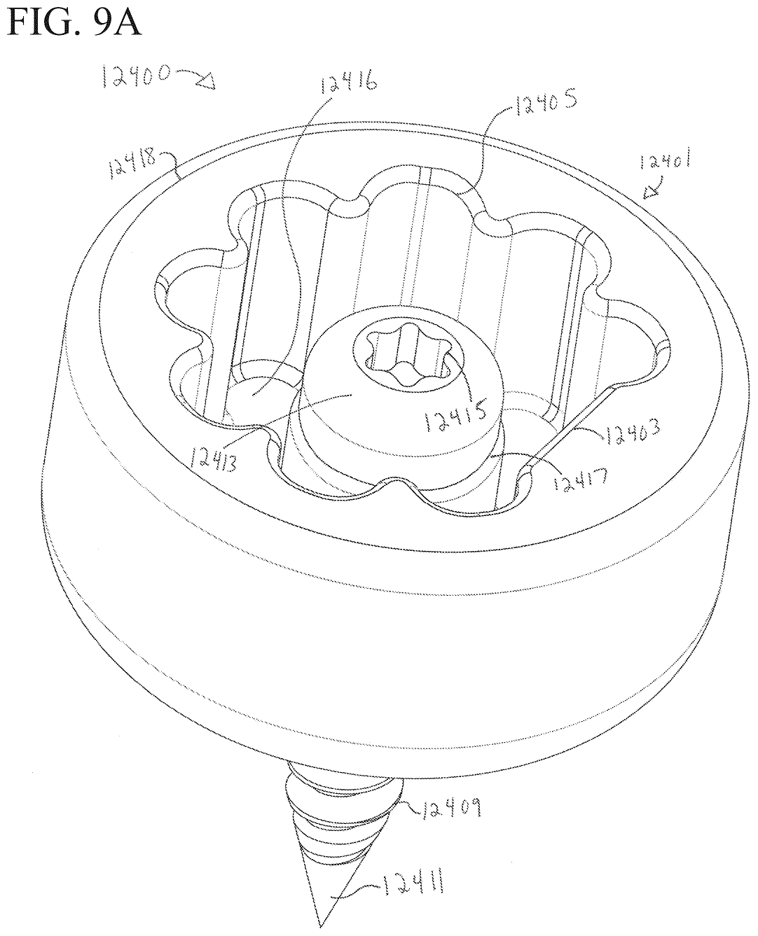

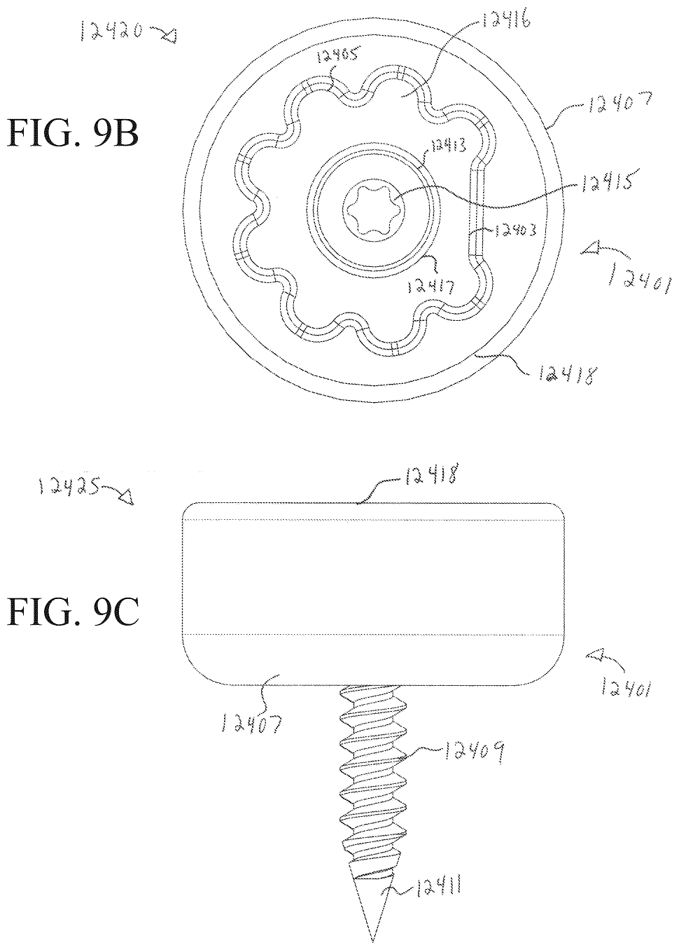

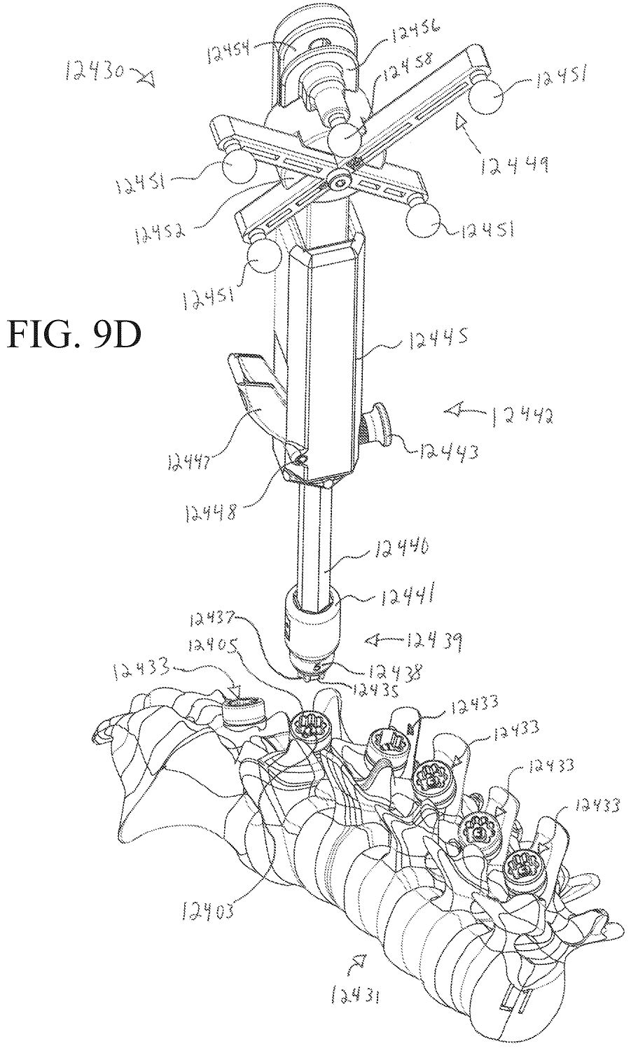

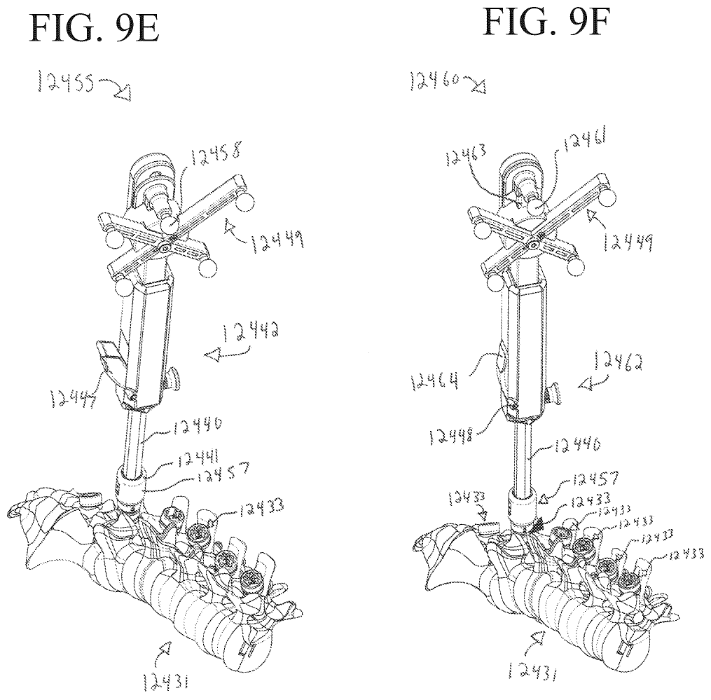

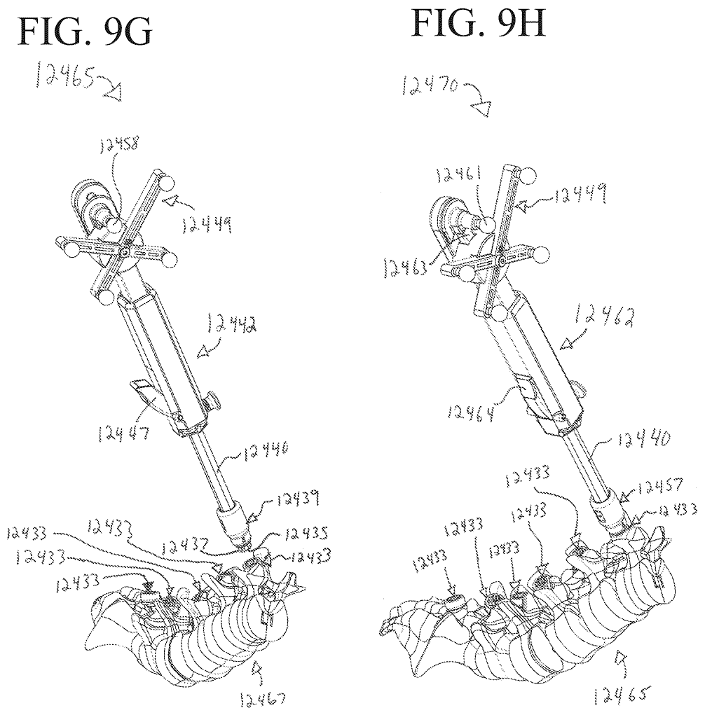

In some embodiments, FIG. 9A illustrates a perspective view of a bone-mounted fiducial device 12401 with a female mating interface that contains an internal male depth-stop mating interface 12416 for engaging with a 3D-tracked tool with an end-effector that contains a similar mating interface. In some embodiments, the bone-mounted fiducial 12401 has a uniquely-defined depth of engagement set by the difference in heights between the screw-head 12413 and the depth-stop mating interface 12416 which is registered by the 3D-tracked probe with complementary mating surfaces and an internal actuating trigger shaft that is depressed via the screw-head 12413 of the bone-mounted fiducial. In some embodiments, the bone-mounted fiducial 12401 can be registered by a 3D-tracked probe with a probe tip adapter 12439 that features the complementary mating surfaces for engaging the bone-mounted fiducial. In some embodiments, FIG. 9B illustrates a top view of the bone-mounted fiducial device 12401. In some embodiments, the fiducial mating interface comprises a torx curved surface and a flat-edge surface to enable registration of the unique orientation of the fiducial by a 3D-tracked tool (not shown). In some embodiments, FIG. 9C illustrates a side view of the bone-mounted fiducial device 12401 that is described previously in relation to FIGS. 9A-B.

In some embodiments, FIG. 9D illustrates a perspective view of a 3D-tracked probe 12442 about to engage with a bone-mounted fiducial device 12433 in a unique trajectory and orientation. In some embodiments, the observed size of the fiducial device 12433 in FIG. 9D does not reflect the end sizing of the manufactured device which is approximately 10% of the seen sizing footprint. In some embodiments, when the user is registering a vertebra of the spine, the 3D-tracked probe 12442 and its linked probe tip adapter 12439 with embedded flat mating surface 12435 and torx-head mating surface 12437 that mates uniquely with the bone-mounted fiducial female mating surface. In some embodiments, FIGS. 9E-9F illustrates the process of mating the 3D-tracked probe 12442 to a bone-mounted fiducial device 12433 and then activating the TMSM 12461 via depressing the trigger button 12464 and actuating the TMSM backing mount 12463. In some embodiments, FIGS. 9G-9H illustrate some embodiments of the invention for use as a rapid re-registration system for updating the 3D position and orientation of initialized vertebrae relative to their individually engaged bone-mounted fiducials 12433. In some embodiments, after the spine 12465 has been corrected into a healthier shape, a 3D-tracked probe 12462 is used to register the new 3D conformation of the spine 12465.

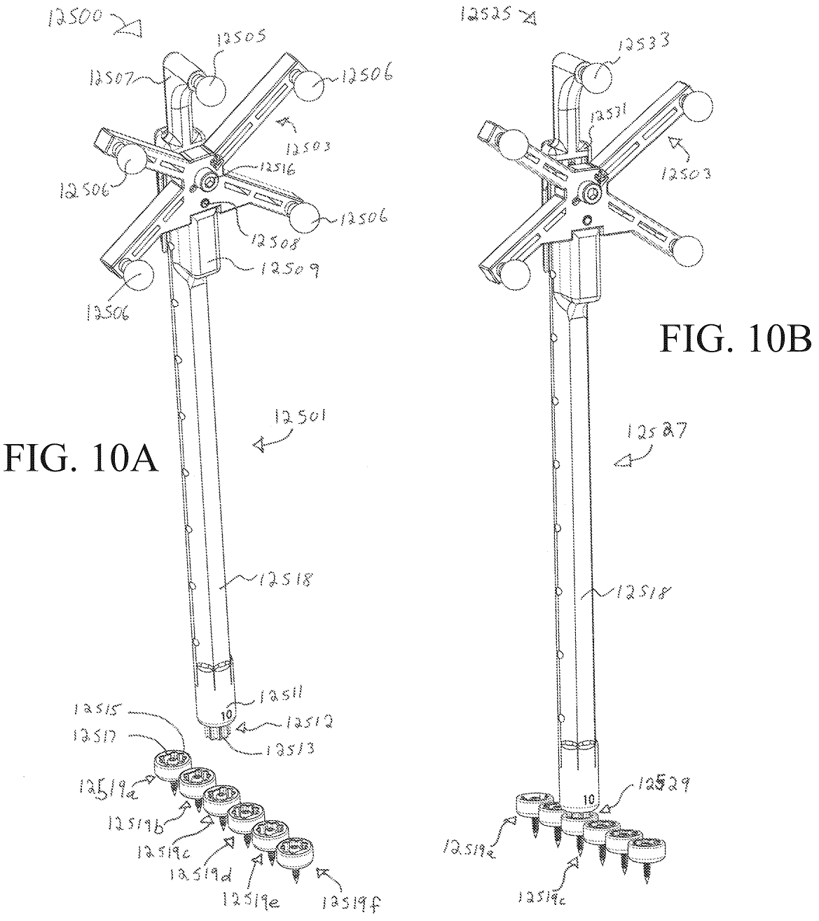

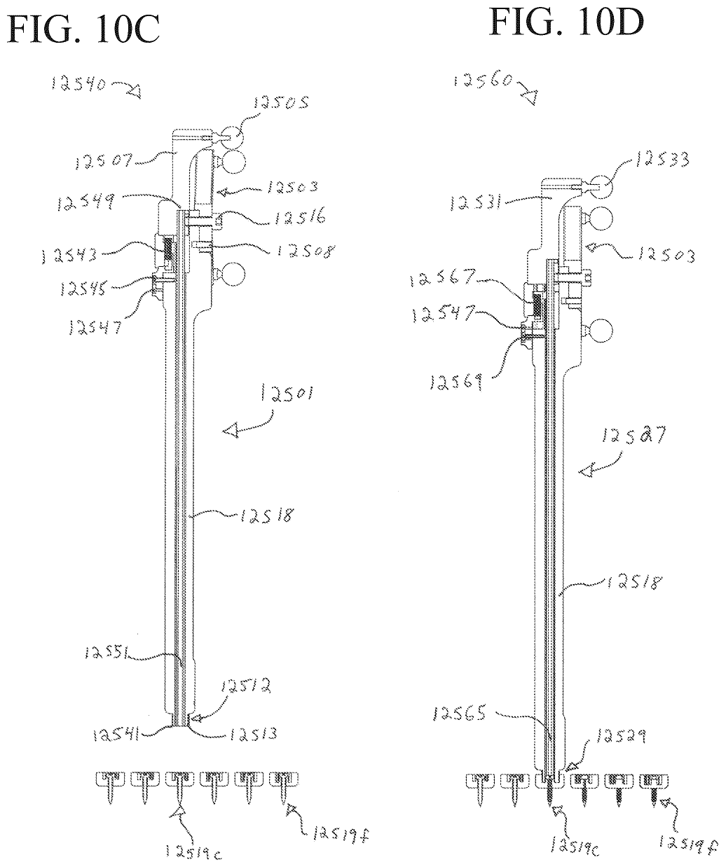

In some embodiments of the invention, a bone-mounted fiducial device with an internal protrusion, as described previously in relation to FIGS. 9A-9H, is registered via a 3D-tracked probe with a self-triggering mechanism that automatically activates upon fully engaging and mating with the mating features between the fiducial and the probe tip. In some embodiments, FIG. 10A illustrates a perspective view of a 3D-tracked probe 12501 with a probe shaft 12518 that encloses an internal shaft (not shown) that is rigidly linked to the TMSM 12505 via the TMSM sliding mount 12507. In some embodiments, when the male mating features 12512 of the 3D-tracked probe 12501 are fully engaged with a bone-mounted fiducial 12519c in a specific orientation in which the mating features align during engagement, the internal shaft 12551 (not shown) is then depressed via the male protruding screw profile base within the internal features of the bone-mounted fiducial. In some embodiments, the internal shaft of the probe cannot be depressed unless the mating features between the fiducial and probe are perfectly aligned to each other. In some embodiments, FIG. 10B illustrates a 3D-tracked probe 12527 once fully engaged with a bone-mounted fiducial 12519c, after which the TMSM 12533 is actuated into an active state.

In some embodiments, FIGS. 10C-10D illustrate a side cross-sectional view of the mating process of the 3D-tracked probe 12527 engaging with a bone-mounted fiducial 12519c, and in the process actuating the internal sliding trigger shaft 12565 which is spring-loaded via a mechanically-linked compression spring 12567 and is also linked to the TMSM sliding mount 12531 that actuates the TMSM 12533 into an active state. In some embodiments, the system automatically identifies which bone-mounted fiducial devices are being registered by analyzing the depth of triggering engagement registered by the 3D-tracked probe 12527. In some embodiments, the greater depth of engagement shown in the far-right bone-mounted fiducial 12519f will signal a different fiducial identity to the system than engaging the third bone-mounted fiducial 12519c with a shallower depth of engagement of the internal sliding trigger shaft 12565.

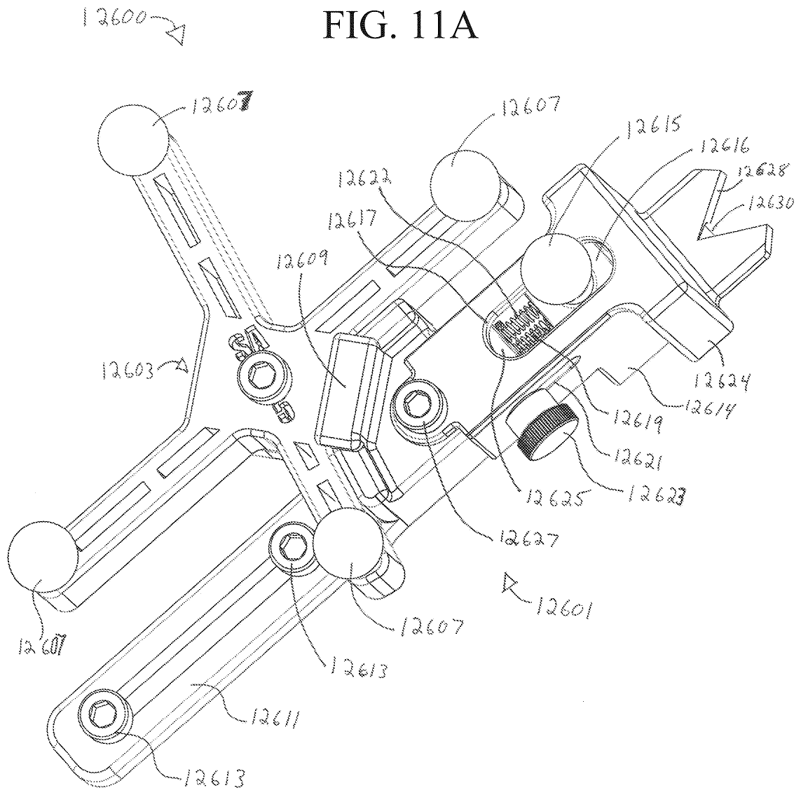

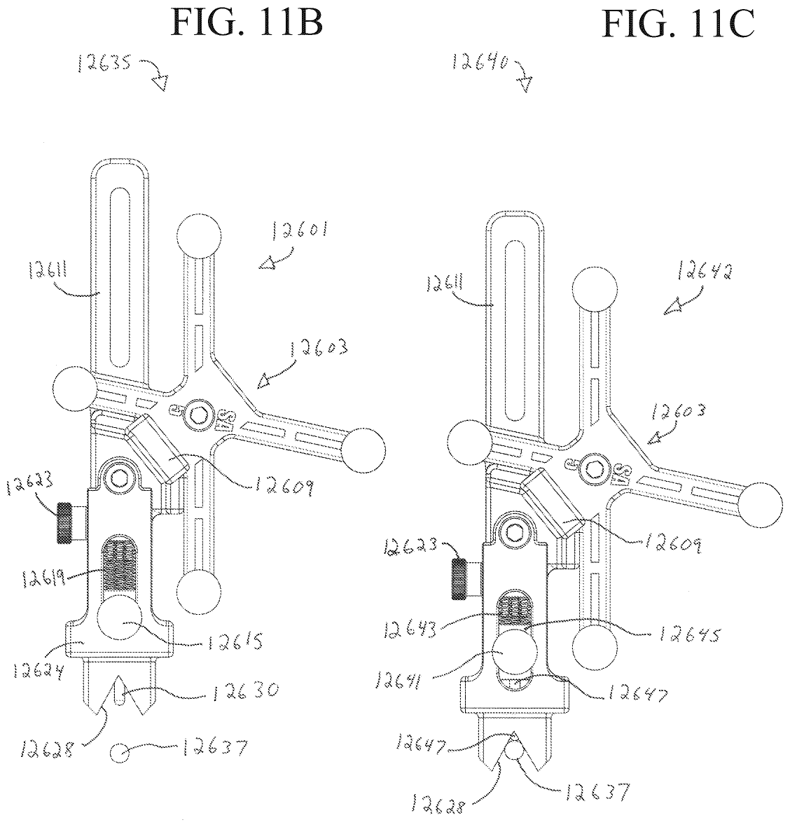

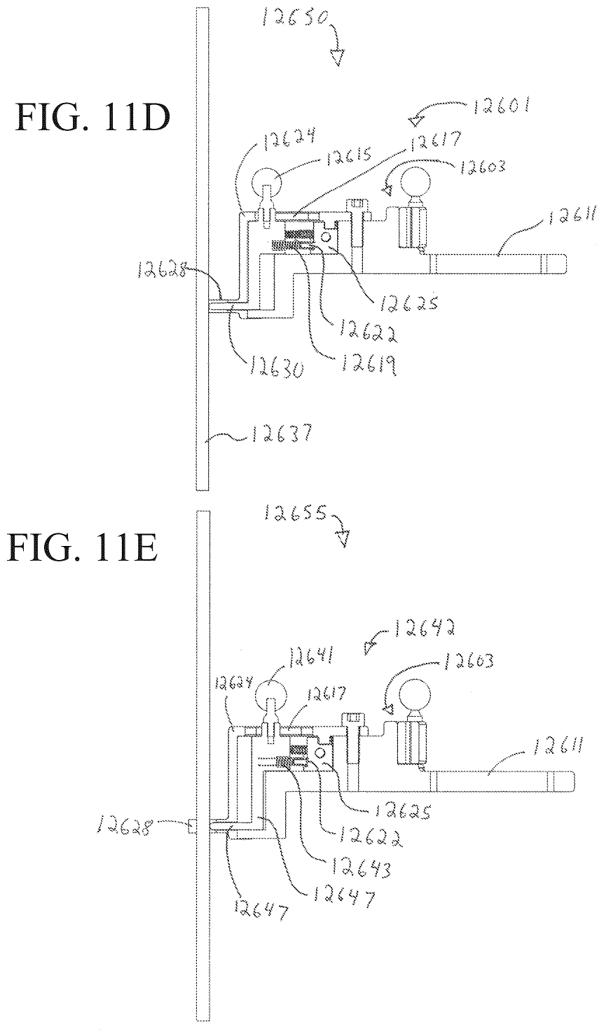

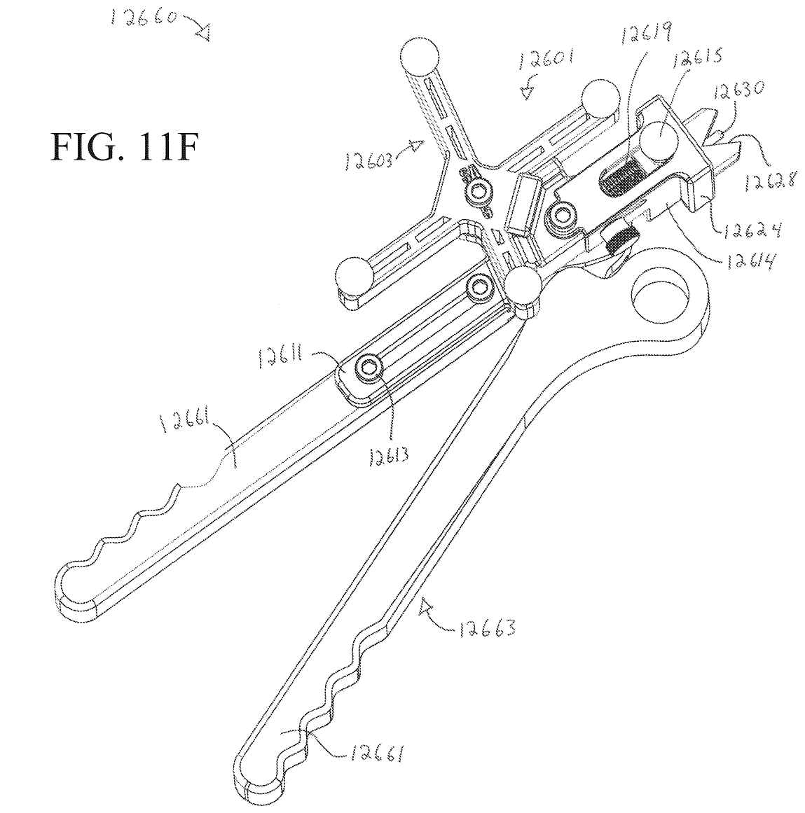

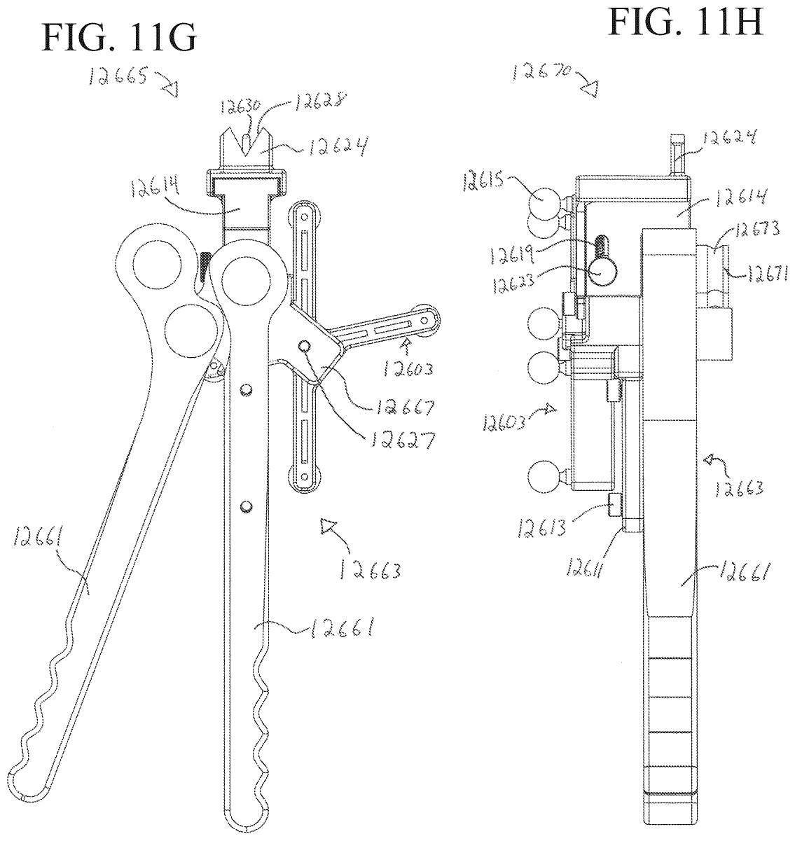

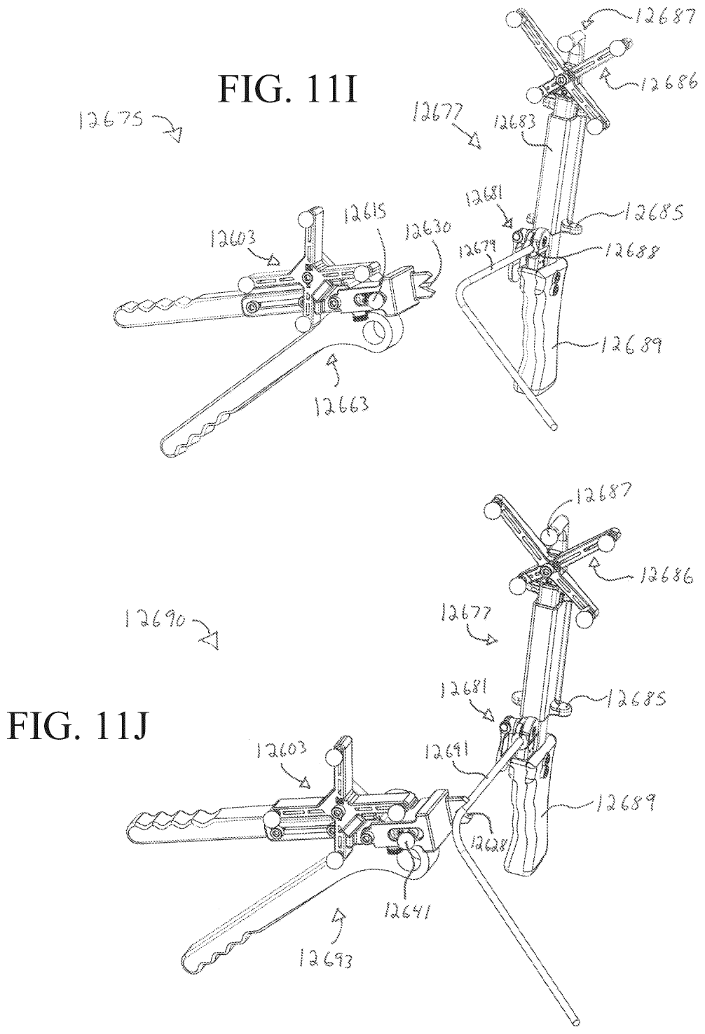

In some embodiments of the invention, a system of 3D-tracked tools is used to register the 3D conformation of a spinal rod implant throughout the rod contouring process. In some embodiments of the invention, a rod is rigidly fixed within a 3D-tracked end cap device and then a 3D-tracked slider device engages the rod and slides along its external contour. In some embodiments, as the 3D-tracked slider device traces the 3D contour of the rod, the 3D-tracked end cap is constantly updating the location and pose of the reference coordinate axes for the rod contour registration. In some embodiments, FIG. 11A illustrates one example embodiment of the 3D-tracked slider device 12601 in which the tracked slider assembly can mount onto a standard universal rod bender for case-of-use and rapid integration in the current workflow of iteratively contouring the shape of the rod implant until its shape is satisfactory to the user. In some embodiments, the 3D-tracked slider comprises a DRF 12603, made of three or more 3D-tracked markers 12607, that is attached to an aligning wall mount 12609 that is connected to a rod bender mounting interface 12611 that is also connected to a trigger offset L-shape arm 12614 that places the triggering mechanism off-axis from the DRF to minimize the device profile protruding from the rod bender top boundary. In some embodiments, within the trigger offset arm 12614, there is a spring-loaded TMSM 12615 that is attached to a sliding mount 12616 that is linked to a series of compression springs 12619 that are retained in place via spring guide protrusions 12622 within a spring mount 12625 that is fixed in-place at a certain distance from the TMSM sliding mount 12616 via a tensioning screw 12623 within a slot 12621 that determines the distance between the sliding mount 12616 and the spring mount 12625. In some embodiments, the TMSM 12615 trigger mechanism is actuated via a spring-loaded plunger 12630 that protrudes slightly out of the fork body and is surrounded by a rod-engaging wall interface 12628.

In some embodiments, FIGS. 10B-10C illustrate a top view of the 3D-tracked slider device 12601 in the process of engaging with the surface contour of a spinal rod implant 12637. In some embodiments, as the 3D-tracked slider 12642 engages with the rod 12637 within the fork surface interface 12628, the spring-loaded plunger 12647 is depressed, which actuates the TMSM 12641 into an active state by compressing the spring 12643 of the trigger mechanism. In some embodiments, as the TMSM 12641 actuates away from the fork surface 12628 and moves closer to the DRF 12603, the system automatically analyzes if the TMSM 12641 has passed a 3D location threshold defined in the system to indicate an active tool state for registering the 3D contour of the rod. In some embodiments, by inputting the known diameter of the rod, the system can automatically adjust the 3D register of the contour of the rod to represent the center of the rod as opposed to the external surface. In some embodiments, FIGS. 10D-E display side, cross-sectional views of the 3D-tracked slider device 12601 in the process of mating with a rod 12637 within the fork interface 12628.

In some embodiments, FIG. 11F illustrates a perspective view of the 3D-tracked slider device, as described previously in relation to FIGS. 11A-11E, attached to the back surface of a standard rod bender 12663. In some embodiments, the 3D-tracked slider device triggering axis is in-line with one of the rod bender handles 12661 and thus enables rod contour registrations to occur about the central axis of the rod bender, and thus also enable the user to register the contour of the rod with either a left or right hand holding the 3D-tracked slider. In some embodiments, FIGS. 11G-11H illustrate a front and side view of an example embodiment of the assembly of the 3D-tracked slider device 12601 attached to a rod bender 12663. In some embodiments, the user can iteratively register the 3D contour of the rod and then adjust the contour on the opposite side of the rod bender with the contour rollers 12671 and rod-engaging interfaces 12673.

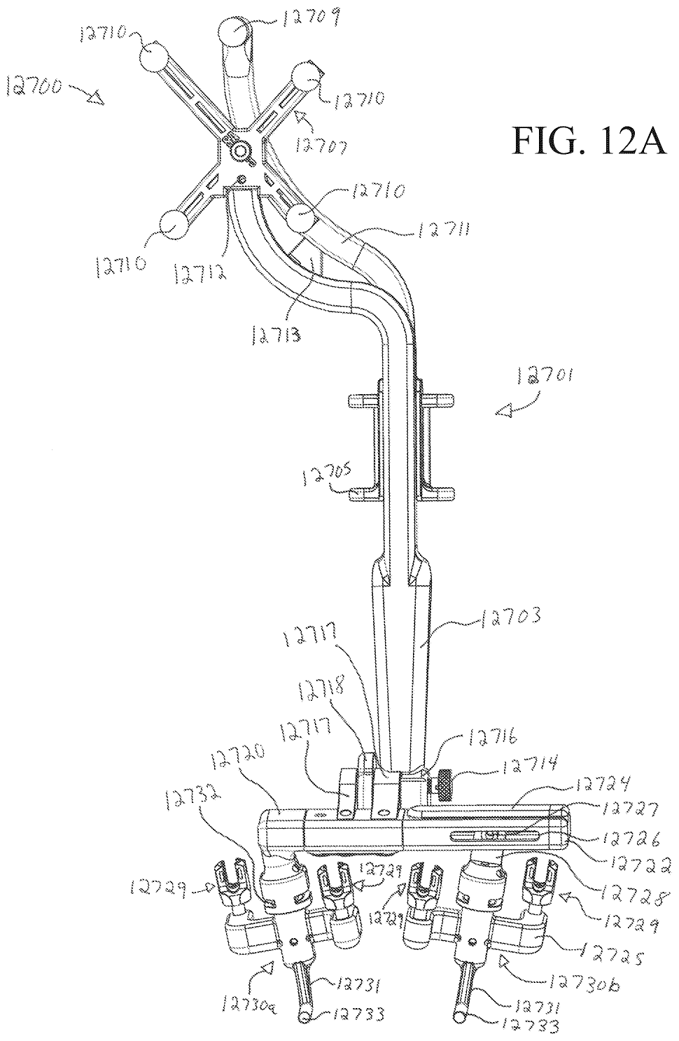

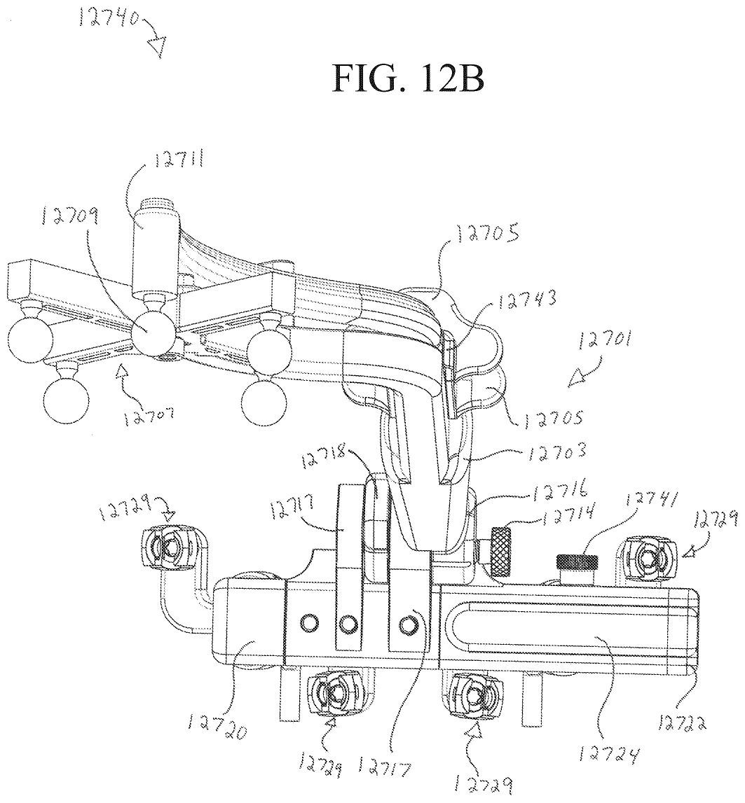

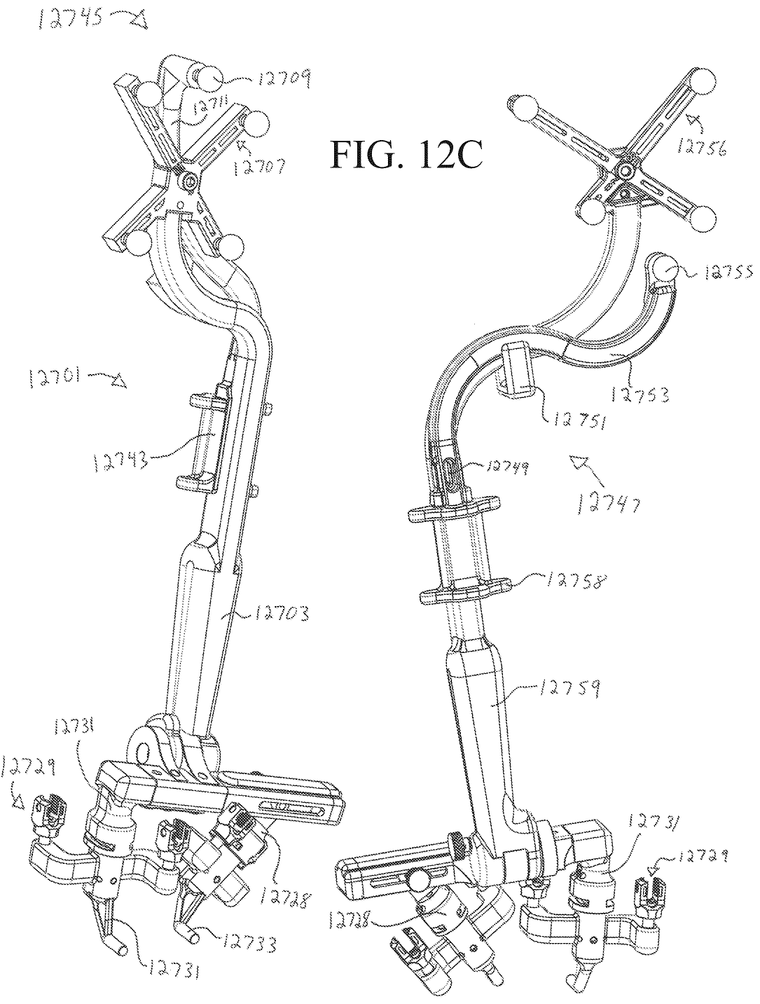

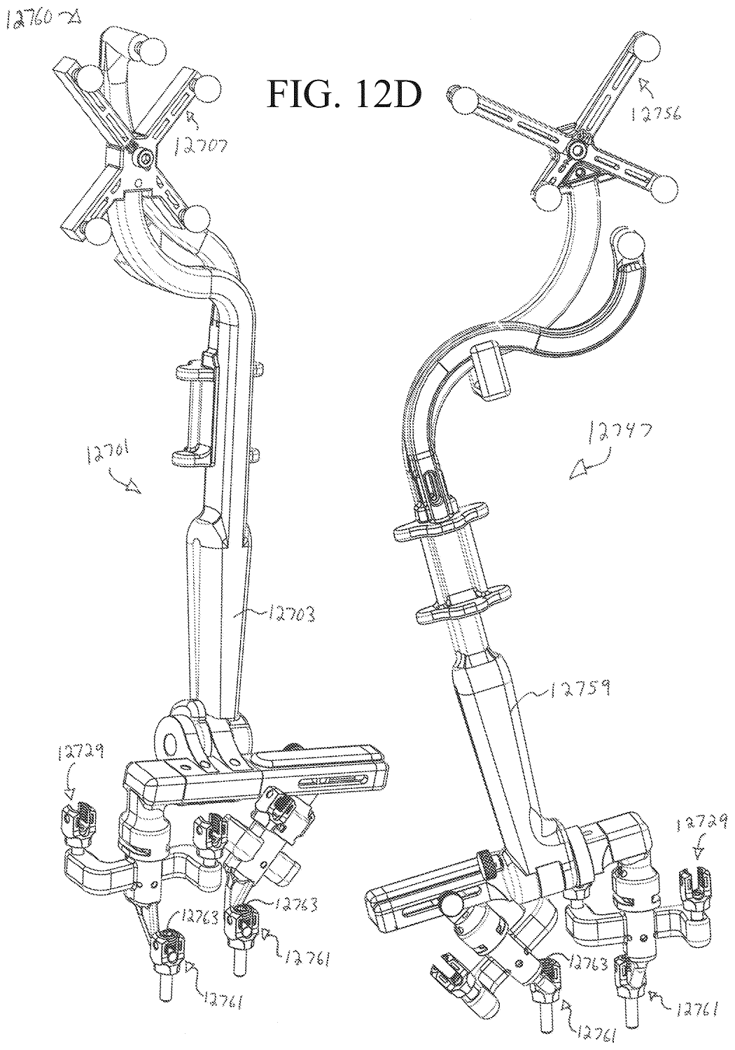

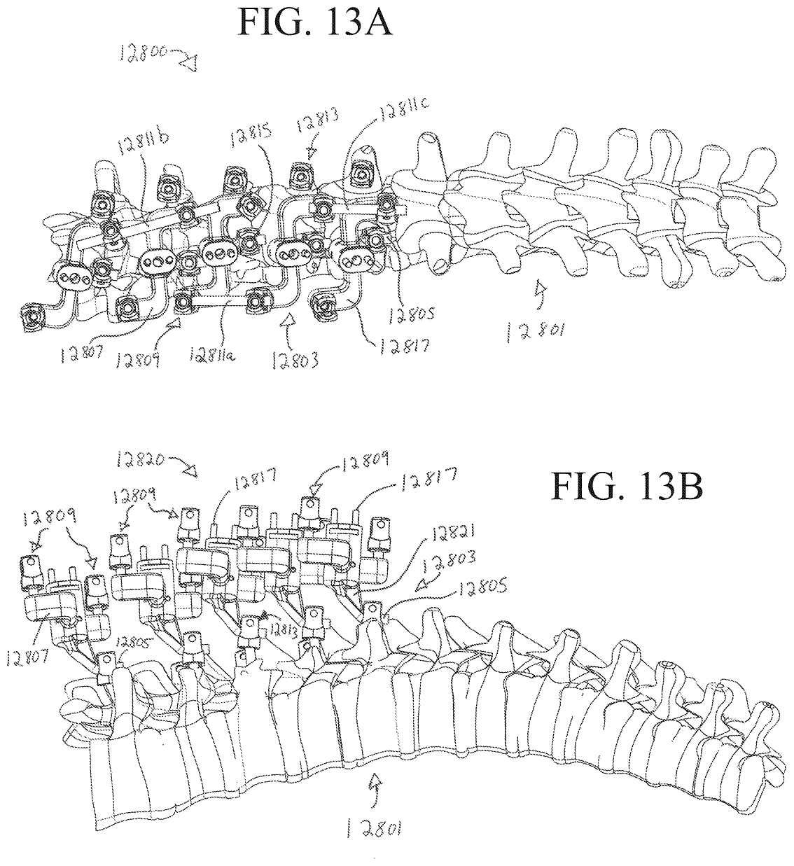

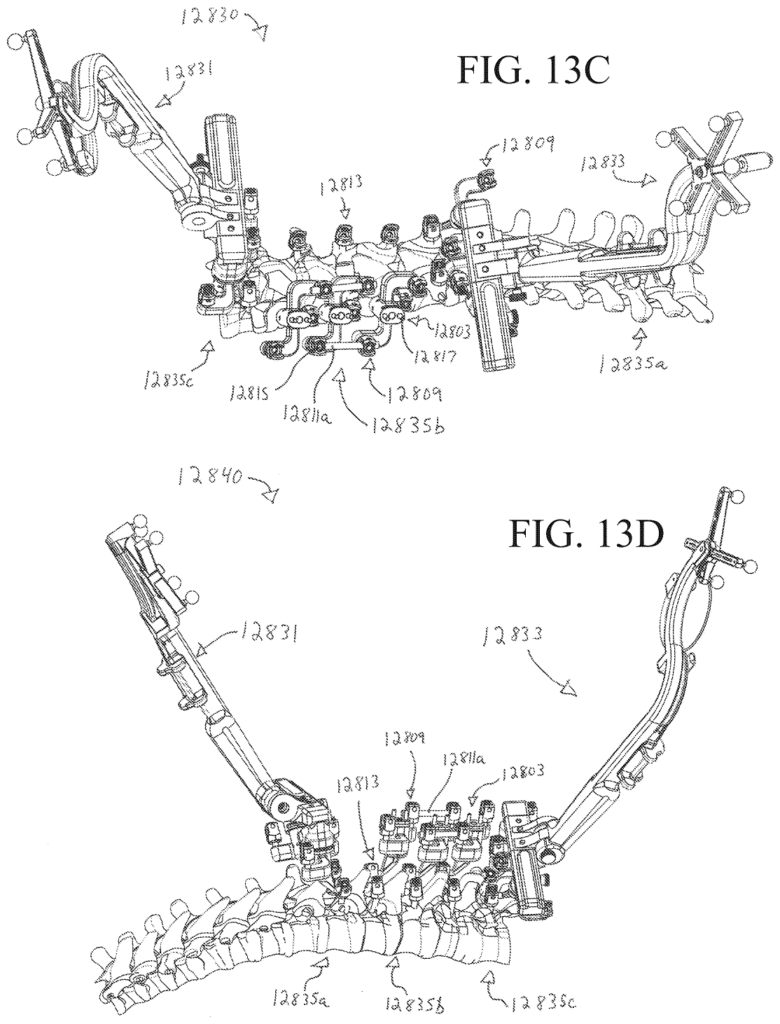

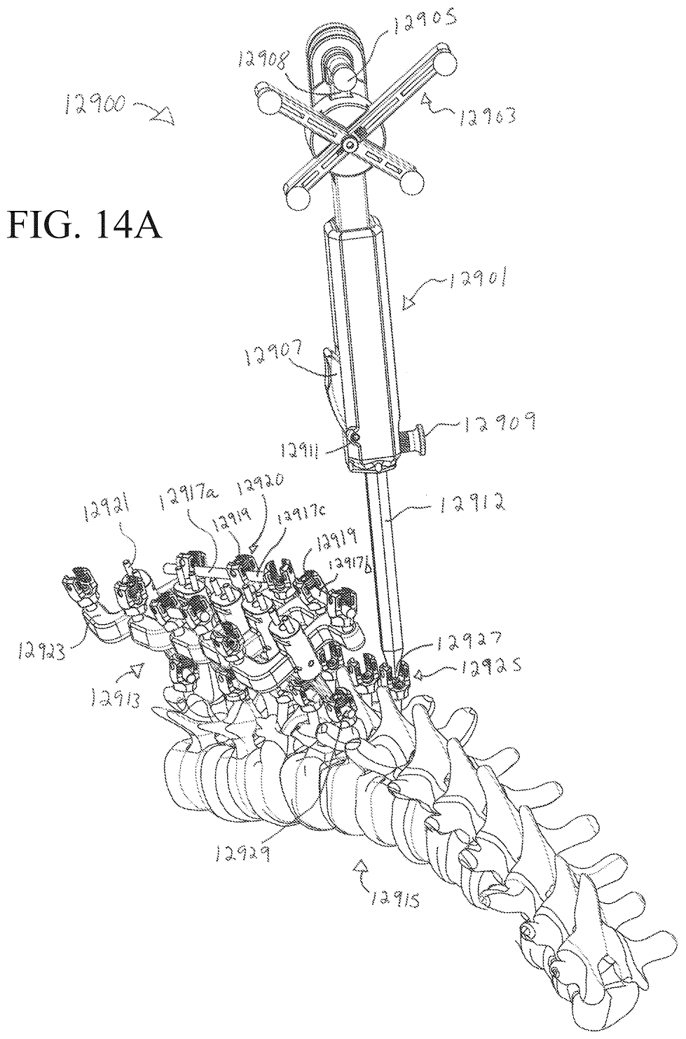

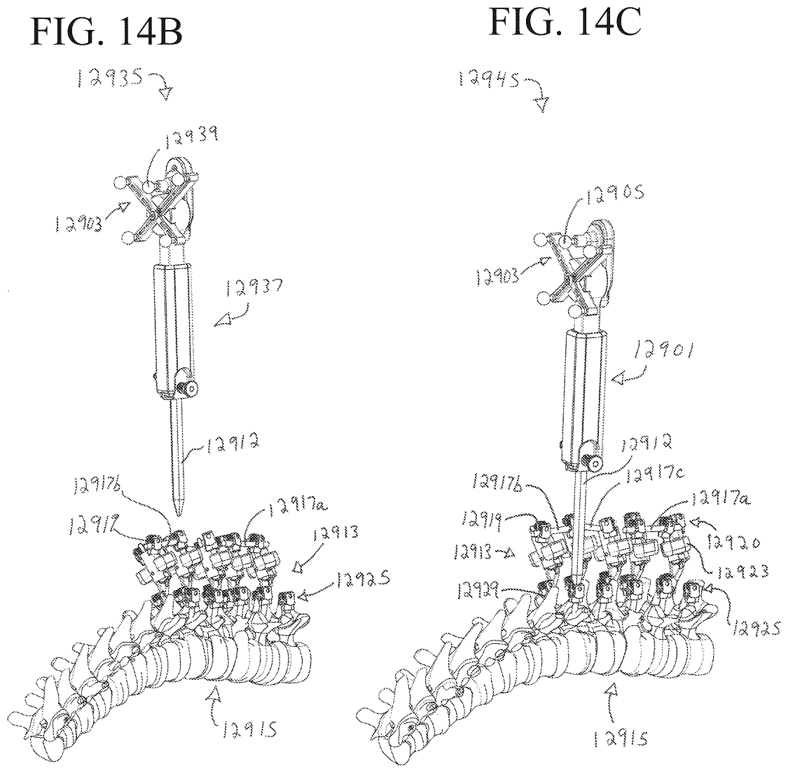

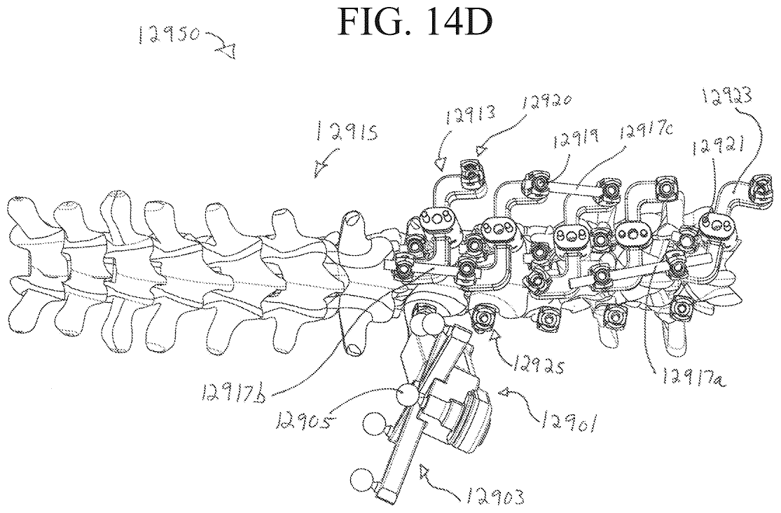

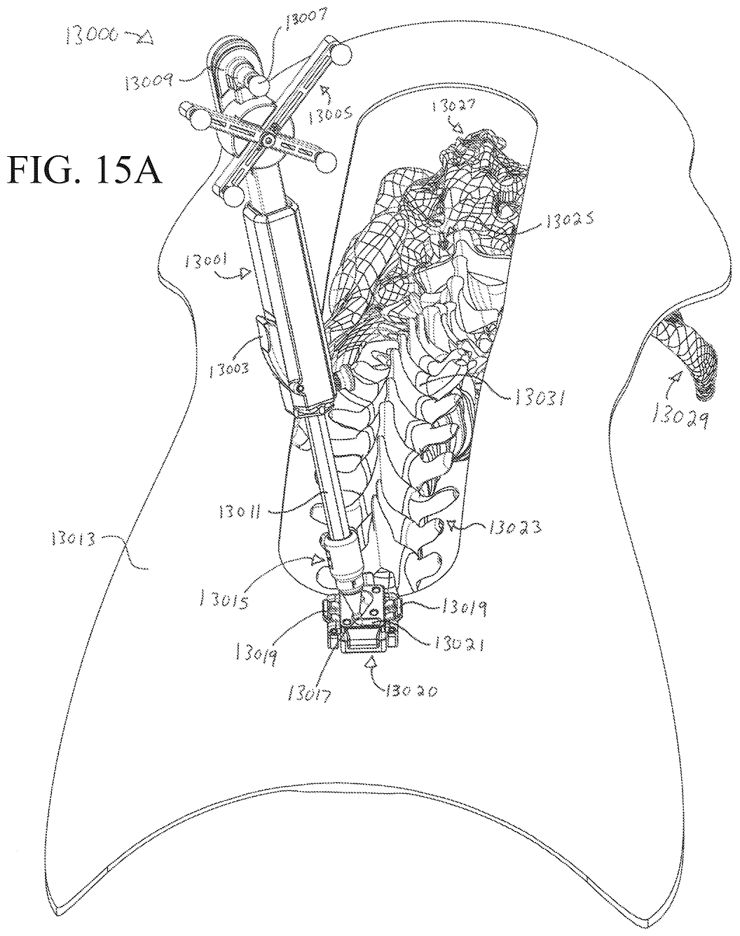

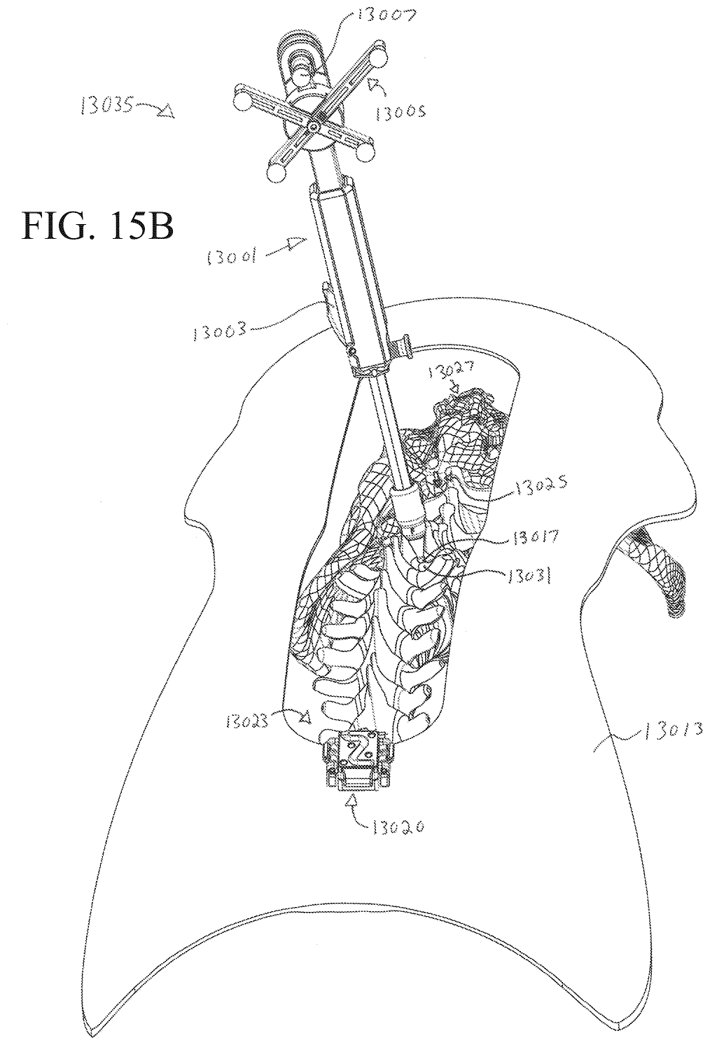

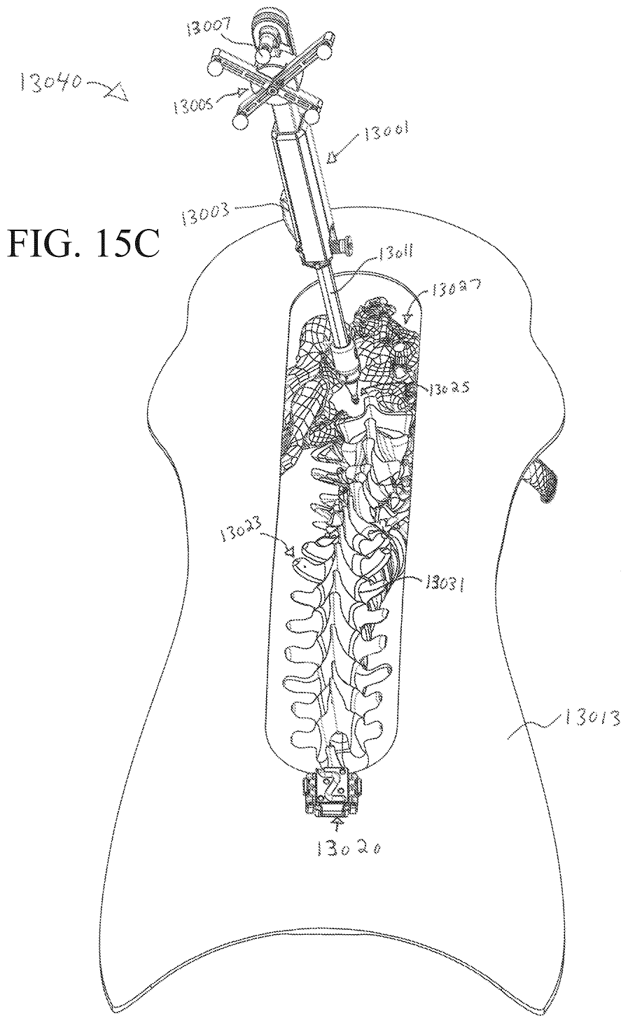

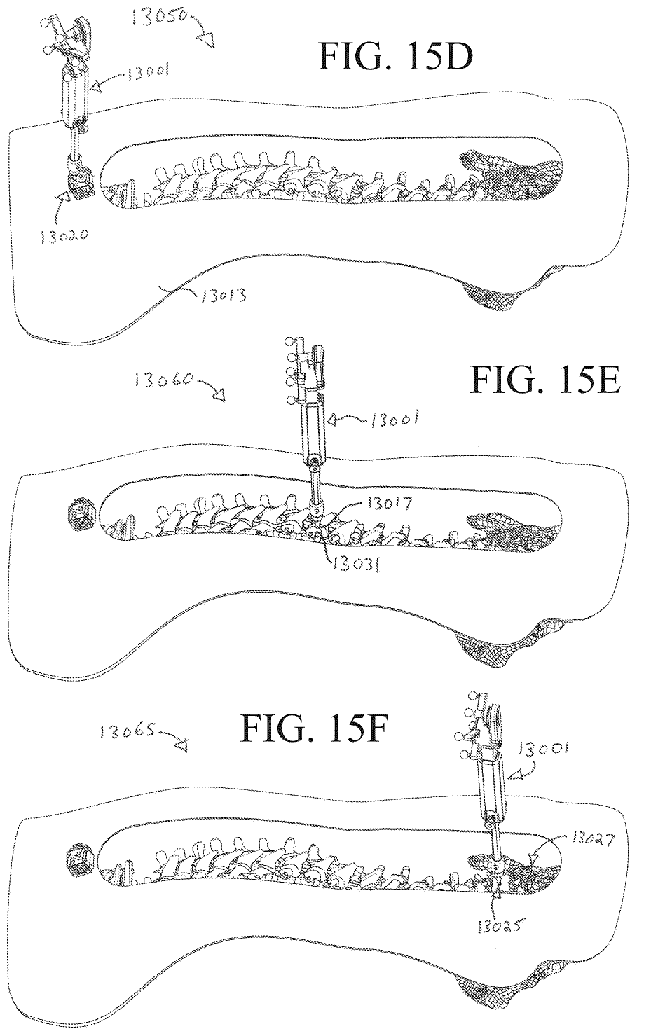

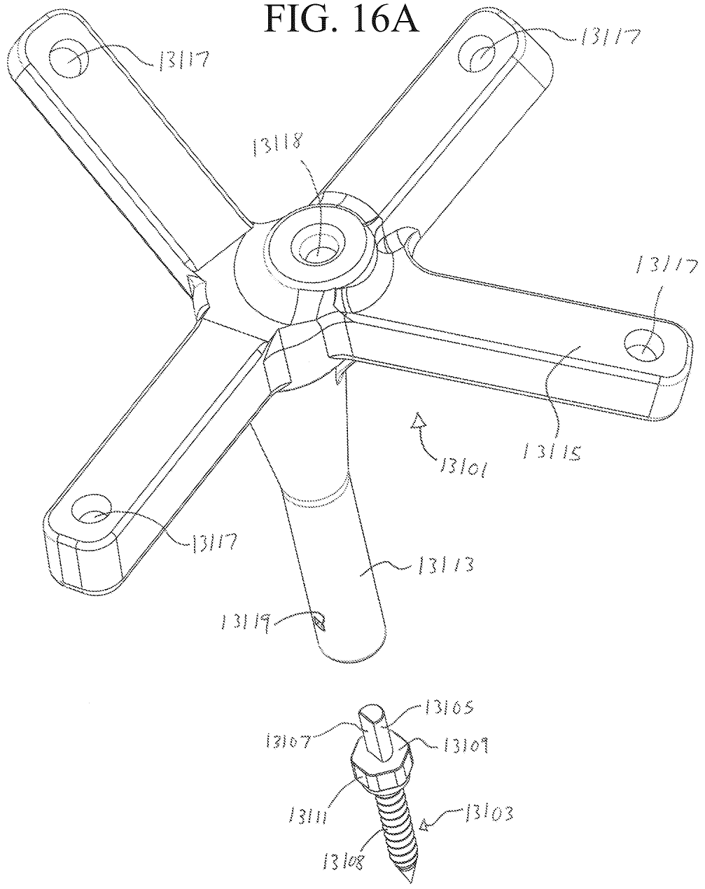

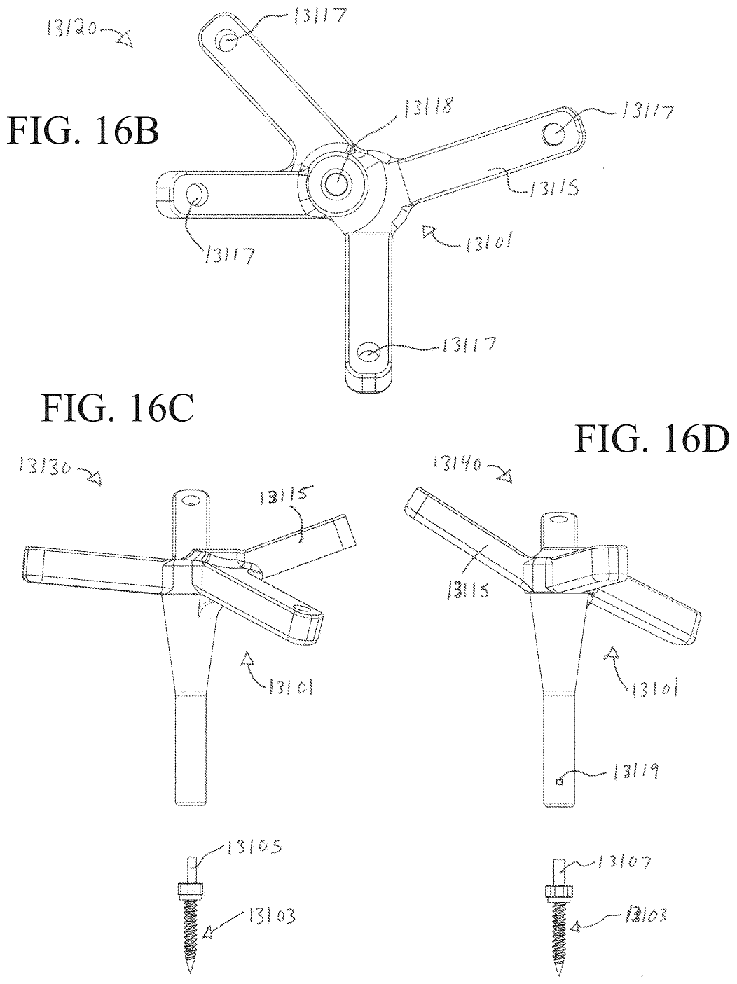

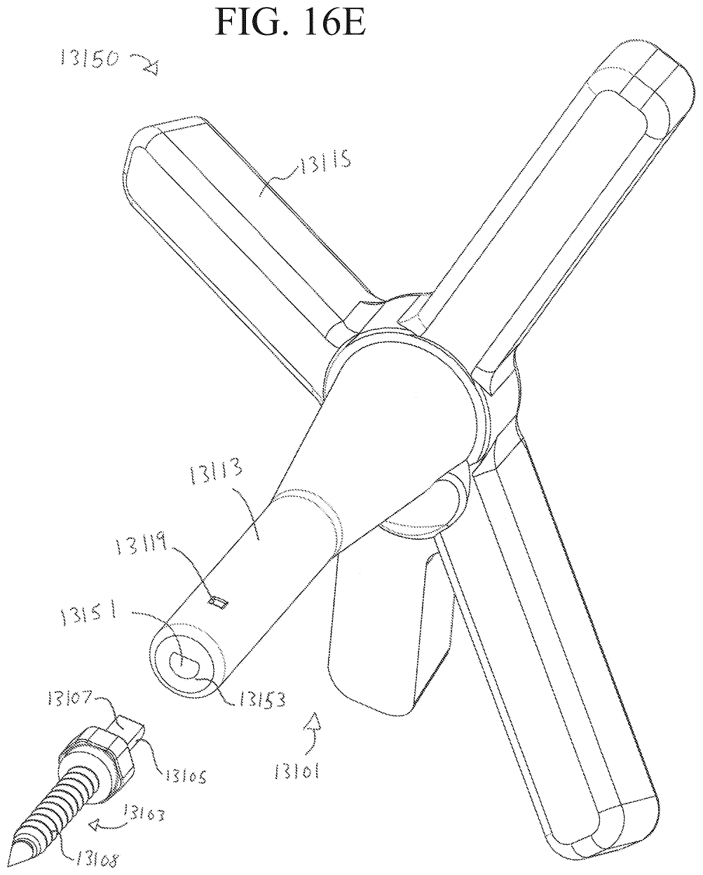

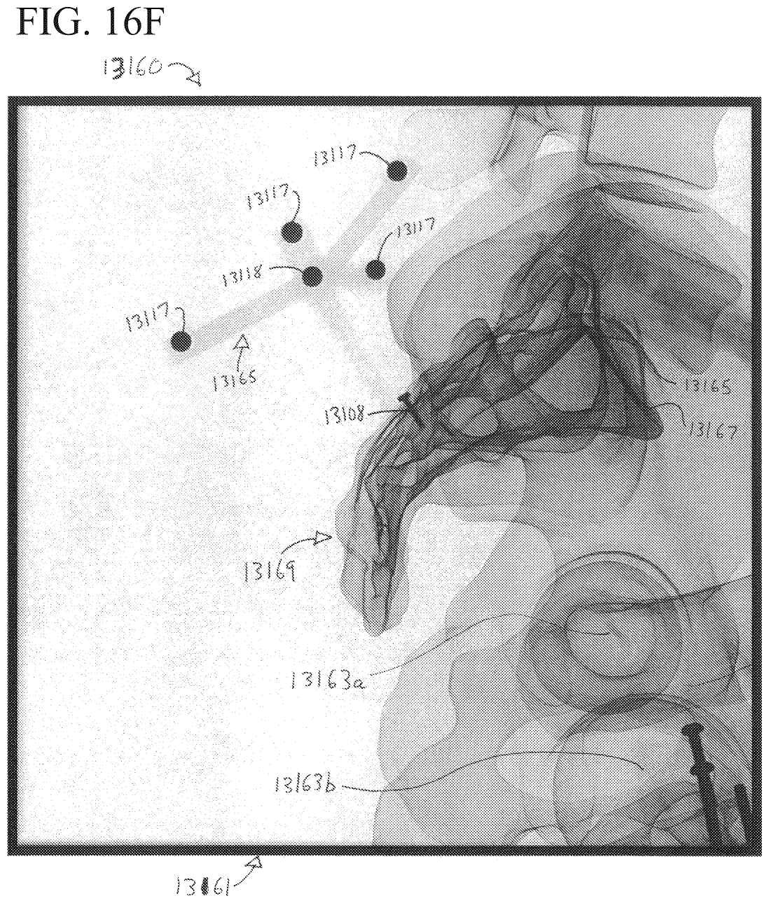

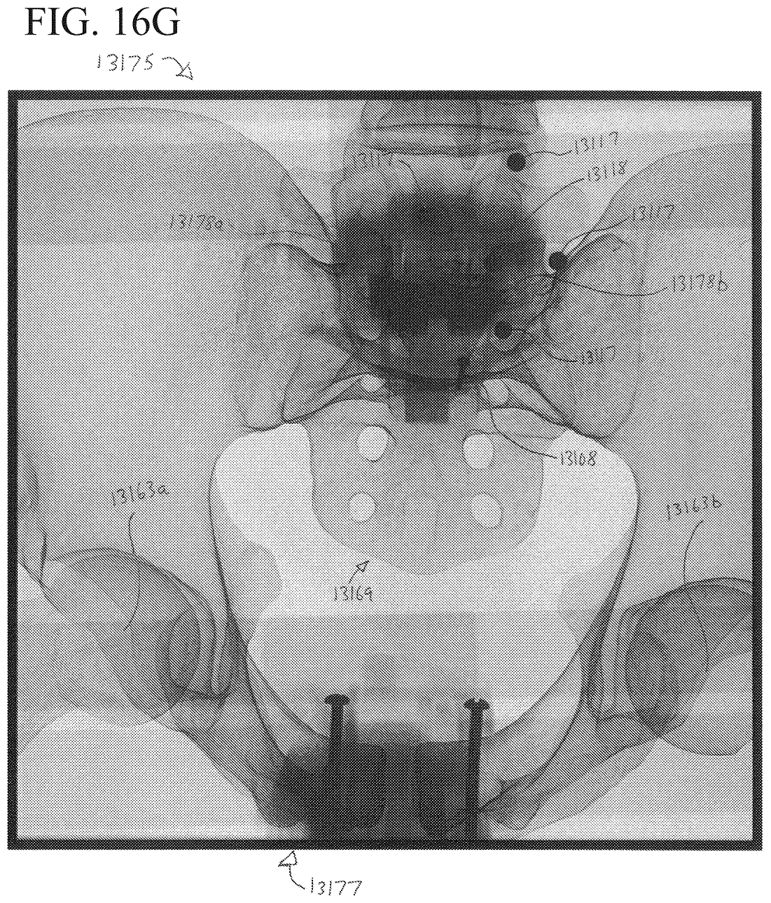

In some embodiments, FIGS. 111-11J illustrate perspective views of the process of registering the 3D contour of a rod during the contouring process via engaging the spring-loaded plunger 12630 against the rod 12679 with the 3D-tracked, slider-equipped rod bender 12693, which is tracing the contour of the rod 12691 while it is being rigidly fixed in place by the clamping mechanism 12681 of the 3D-tracked end cap device 12677. In some embodiments, the end cap 12677 is also equipped with a trigger mechanism 12685 and associated TMSM 12687 to enable the pair of devices to be used to communicate different steps of the rod contouring and registration process. In some embodiments, after a rod tracing registration occurs, the user can click on the end cap trigger 12685 to communicate to the computer that the registration is complete and ready for overlaying and comparison visualizations.

Some embodiments of the invention involve a flexibility and biomechanical analysis system that directly manipulates the conformation of the spine while tracking the position of engaged and nearby anatomical landmarks that are attached to the flexibility assessment devices. In some embodiments, the flexibility assessment devices attach directly to instrumented pedicle screws within the spine via a pseudo-rod component that is compressed with a standard screw cap to convert a polyaxial screw into a monoaxial screw and enable rigid manipulation of the attached vertebrae.

In some embodiments, FIG. 12A illustrates a front view of an example embodiment of a 3D-tracked flexibility assessment device 12701. In some embodiments of the invention, the flexibility assessment device 12701 includes a 3D-tracked DRF 12707, an actuating trigger arm 12711 that is depressed via trigger buttons 12705 that actuate the position of a TMSM 12709. In some embodiments of the invention, the trigger arm 12711 and device handle 12703 are curved in shape to enable multiple flexibility assessment devices in the surgical site to be simultaneously visualized by a 3D-tracking camera without obstructing line-of-sight during a flexibility assessment. In some embodiments of the invention, the device handle 12703 can be adjusted in angle relative to the width-adjustment mechanism 12624 via a spring-loaded plunger 12714 that releases and engages predefined angular detents within the handle mount side walls 12717. In some embodiments of the invention, the device comprises a width-adjustment mechanism 12624 that connects two side arm devices (12730a, 12730b), one attached to the primary device via a fixed shoulder 12720 and the other via an adjustable shoulder 12728 that can adjust the inter-side-arm distance and angle for matching the distance and angles between pedicle screw tulips instrumented into a vertebra of interest. In some embodiments, the adjustable shoulder 12728 slides within the width adjustment channel 12726 and can rotate about the width adjustment pivot 12727 that can be fixed in-place via tightening width-adjustment knob 12741 (not shown). In some embodiments, the device side arms engage with the pedicle screws of the engaging vertebra via a pair of pseudo rod mating interfaces 12733 that insert into the tulip of the pedicle screws and can be compressed in-place via standard screw caps. In some embodiments, to enable more rigid structural support during manipulation of vertebrae, the side arm devices (12730a, 12730b) can be reinforced with gusset structures 12731 between the side arm and the pseudo rod interface 12733. In some embodiments, after the side arms are rigidly attached to the pedicle screws of interest and the manipulating spine conformation has been achieved, the side arm devices (12730a, 12730b) can be detached from the primary device 12701 via removal of a retaining clip (not shown) that slides within the shoulder clip groove 12732. In some embodiments, the side arm devices (12730a, 12730b) are equipped with device pedicle screws 12729 that are used to connect a rigid rod connector (not shown) between two or more flexibility assessment devices once a desired alignment or 3D spinal conformation has been achieved with the assessment devices 12701.

In some embodiments, FIG. 12B illustrates a top view of the flexibility assessment device 12701. In some embodiments, the positioning and shape of the side arm devices (12730a, 12730b) enable for the device pedicle screws 12729 to all be accessible and not occluded from above for access of inter-tool rod placement. In some embodiments, FIGS. 12C-12D illustrate perspective views of two flexibility assessment devices (12701,12747) in an example embodiment of arranging the orientation of devices within the surgical site and maintaining visualization of both DRFs (12707,12756) during flexibility assessments. In some embodiments, FIG. 12D illustrates a perspective view of the flexibility assessment devices (12701,12747) engaged into pedicle screws 12761 and rigidly linked in-place via screw caps 12763 that are tightened onto the pseudo rod mating interface 12733.

In some embodiments of the invention, two or more flexibility assessment device handles can be rigidly fixed in-place, and subsequently the nearby vertebrae maintain a desired conformation, via the placement of inter-tool connecting rods, and then the device handles can be detached from the side arm devices via removal of a retaining clip or connecting mechanism. In some embodiments of the invention, multiple interconnected side arm devices can be daisy-chained along the spine to provide a large construct that has been individually measured and locked in-place via rigidly linked, and then detached, flexibility assessment devices.

In some embodiments, FIGS. 13A-13B illustrate a top and side view of an example embodiment of the invention that include multiple engaged, connected side arms 12803 that are linked to one another via multiple inter-tool connecting rods (12811a, 12811b, 12811c). In some embodiments, the side arm device 12803 includes alignment pins 12817 that help rigidly, reliably link the side arm device with their respective flexibility assessment device (not shown).

In some embodiments, FIGS. 13C-13D illustrate a top and side view of another example embodiment of the invention that demonstrates that flexibility assessment devices (12831, 12833) can be rigidly engaged with empty pedicle screw tulips on surrounding sides of the spine (12835a, 12835c) around engaged, connected side arms that are rigidly holding the conformation of a middle segment of the spine 12835b. In some embodiments, the system can continue evaluating the range of motion and 3D alignment of engaged vertebrae not rigidly linked to the fixed construct to enable adding additional measurement-approved fixed segments to the construct or to assess that the final global alignment of the full construct is as desired relative to the operative goals.

In some embodiments of the invention, the 3D location pedicle screw tulips can be registered to provide input to automated, manual, or assisted rod contouring systems as to the desired 3D contour of the final rod implant. In some embodiments, FIG. 14A illustrates a perspective view of an example embodiment of the system in which a 3D-tracked probe 12901 registers the 3D location of pedicle screw tulips 12925 via inserting the probe tip 12927 within the tulip cavity and depressing the trigger button 12907 of the probe which actuates the TMSM 12905 via depressing the TMSM sliding mount 12908. In some embodiments of the invention, the spine 12915 is instrumented with pedicle screws 12925 and a select segment of the spine is rigidly fixed via a series of engaged, inter-connected side arm devices 12913 that are connected via inter-tool connecting rods (12917a, 12917b, 12917c). In some embodiments, the engaged side arms are linked to pedicle screws on the right side of the patient, and the contralateral array of pedicle screw tulips are empty and able to be localized in 3D via acquisitions of the 3D-tracked probe 12901. In some embodiments, FIGS. 14B-14D illustrate side and top views of an example embodiment of the system that depicts the process of inserting the probe tip of the 3D-tracked probe 12937 and activating its TMSM 12905 to signal to the computer to acquire the pedicle tulip 3D location. In some embodiments, this system of acquiring the 3D locations of contralateral pedicle screw tulips can be inputted into a feedback system that overlays the current registration of a contoured spinal rod relative to the contour of the spine and the conformation of screw tulips in sagittal and coronal anatomical projections, as depicted in example system embodiments shown in FIGS. 134A-134H. In some embodiments, the 3D registered location of the pedicle tulips can be inputs to automated or assisted rod bending devices. In some embodiments, one useful feature of this system is the ability to provide feedback as to how the current rod contour compares with the current arrangement of pedicle screw tulips, which are already in their final corrected position due to manipulations made by the flexibility assessments devices.

In some embodiments of the invention, a system of a 3D-tracked probe and fiducial devices are used to register the 3D location and orientation of key anatomical landmarks of interest of the spine for the calculation of spinal alignment parameters during surgery. In some embodiments, FIG. 15A illustrates a perspective view of an example embodiment of the system that demonstrates the clinical workflow of the invention. In some embodiments, for regions of the spine that are required or desired inputs for alignment parameter calculations but are outside the surgical site, a skin-mounted fiducial assembly 13020 can be applied over the anatomical landmarks of interest covered by skin tissue 13013 and surgical drapes. In some embodiments, after the skin-mounted fiducial 13020 is initialized via multi-planar X-Ray images or image-guided navigation, the 3D-tracked probe 13001 traces the Z-pattern registration groove 13021 to register the 3D coordinate axes of the fiducial device. In some embodiments, updating the 3D location and pose of the skin-mounted fiducial produces a rigid body transform calculation to convert the image-based initialization of anatomical landmarks with respect to the fiducial axes into 3D-tracking camera coordinates. In some embodiments, tracing the Z-pattern registration groove instantly calculates the up-to-date virtual, 3D location of the T1 vertebral body centroid (not shown; illustrated in FIGS. 133H-I), which is located in the region beneath the skin-mounted fiducial 13020.

In some embodiments, FIG. 15B illustrates a perspective view of the spinal alignment assessment workflow as described previously in relation to FIG. 15A. In some embodiments of the invention, after the skin-mounted fiducial has been registered, the user now traces the exposed bony landmarks along the spine 13023, such as the laminac 13031 via applying a probe tip 13017 of a 3D-tracked probe 13001 to the surface of the spine and tracing along the full exposed range of the spinal column 13023.

In some embodiments, FIG. 15C illustrates a perspective view of the spinal alignment assessment workflow as described previously in relation to FIGS. 15A-15B. In some embodiments of the invention, after the skin-mounted fiducial and the exposed laminae surface have been registered, the 3D-tracked probe 13001 is used to acquire the 3D location and orientation of the implanted, male-mating bone-mounted fiducial device 13025 that is attached to the sacral vertebral body 13027. In some embodiments, the design of the probe tip is such that the spherical ball tip exterior surface can be used for fiducial surface registrations, bony anatomy tracings, as well as mating with male protrusion devices of similar cross-sectional pattern. In some embodiments, FIGS. 15D-15F illustrate side views of the workflow described previously in relation to FIGS. 15A-15C. In some embodiments, any of these three components of the workflow can be conducted in isolation and do not depend on each other in all cases, and in some cases a single component or device may be used multiple times in a particular workflow of assessing spinal alignment, such as having multiple bone-mounted fiducial devices implanted and no skin-mounted fiducials, even without the tracing registration of bony landmarks within the surgical site.

Some embodiments of the invention include an X-Ray adapter device that mates in a unique orientation with a bone-mounted fiducial device. In some embodiments, the X-Ray adapter device contains radiopaque sphere fiducials used for initialization of the location and pose of nearby anatomical landmarks of interest relative to the implanted bone-mounted fiducial device. In some embodiments of the invention, the X-Ray adapter device is used to initialize the 3D location and pose of key anatomical landmarks such as S1 endplate, femoral heads, L5, L1, T10, T9, T4, T1, C1, C7, occiput, etc. to calculate spinal alignment measurements. In some embodiments, FIG. 16A illustrates a perspective of an example embodiment of the X-Ray adapter device 13101 that contains five radiopaque fiducial spheres 13117 within the fiducial body 13115, and a female mating interface (not shown) in a unique orientation. In some embodiments, the arrangement of radiopaque fiducial spheres 13117 involves an asymmetric distribution of spheres in a 3D-offset pattern that produces a unique 3D pose for all rotational views of the sphere arrangement. In some embodiments of the invention, one of the radiopaque fiducial spheres 13118 is located at the centroid of the inter-sphere arrangement of fiducial markers. In some embodiments, the location of the centroid radiopaque fiducial sphere enhances the accuracy of the registration of the fiducial's 3D pose relative to the imaging detector plane and the subsequent initialization of anatomical landmarks in the image that are desired for registration with respect to the coordinates of a 3D-tracking camera. In some embodiments, using the known distribution of radiopaque fiducial spheres (13117,13118) with respect to the origin bottom surface of the adapter device's stem 13113, and along axes in-line and orthogonal to that of the orientation indicator 13119, the registration of the bone-mounted fiducial 13103 via a 3D-tracked probe (not shown) automatically produces the coordinates of the radiopaque fiducial spheres in 3D-tracking camera coordinates. In some embodiments, a 3D rigid body transform can be calculated between the fiducial sphere coordinates relative to the fiducial centroid 13118 in X-Ray image coordinates and the location of the fiducial spheres with respect to the coordinate axes of the 3D-tracking camera. In some embodiments of the invention, the X-Ray adapter device 13101 mates with a male-protruding bone-mounted fiducial device that provides a depth-stop mating interface 13109 for reliable acquisitions of the initialized bony landmarks of interest between the X-Ray adapter device and subsequent 3D-tracked probe acquisitions.

In some embodiments, FIGS. 16B-16D illustrate top and side views of the X-Ray adapter device 13101 in the process of aligning orientations with the mating surfaces (13105, 13107) of the male bone-mounted fiducial device 13103, which are described previously in relation to FIG. 16A. In some embodiments, FIG. 16E illustrates a perspective, underside view of the X-Ray adapter device as described previously in relation to FIGS. 16A-16D. In some embodiments, the female mating interface of the X-Ray adapter device 13101 feature a flat mating interface 13151 and a female cylindrical mating interface 13153.

In some embodiments, FIG. 16F illustrates a Lateral X-Ray image 13161 of the X-Ray adapter device 13165, as described previously in relation to FIGS. 16A-16E, fully engaged onto the male bone-mounted fiducial device 13108. In some embodiments, the unique arrangement of asymmetric radiopaque fiducial spheres (13117,13118) can be clearly visualized without occlusion. Some embodiments of the invention involve user input annotations of bony anatomical landmarks of interest that are to be initialized relative to the fiducial coordinate 13118 of the X-Ray adapter device 13165. In some embodiments, FIG. 16G illustrates an AP X-Ray image 13177 of the X-Ray adapter device 13165, as described previously in relation to FIGS. 16A-16F, along with the key anatomical landmarks of interest for pelvic spinal alignment parameters, including S1 coronal left and right endplate corners, femoral head centroids, and the midpoint of the S1 endplate.

Any of the operations described herein that form part of the invention are useful machine operations. The invention also relates to a device or an apparatus for performing these operations. The apparatus can be specially constructed for the required purpose, such as a special purpose computer. When defined as a special purpose computer, the computer can also perform other processing, program execution or routines that are not part of the special purpose, while still being capable of operating for the special purpose. Alternatively, the operations can be processed by a general-purpose computer selectively activated or configured by one or more computer programs stored in the computer memory, cache, or obtained over a network. When data is obtained over a network the data can be processed by other computers on the network, e.g. a cloud of computing resources.

The embodiments of the present invention can also be defined as a machine that transforms data from one state to another state. The data can represent an article, that can be represented as an electronic signal and electronically manipulate data. The transformed data can, in some cases, be visually depicted on a display, representing the physical object that results from the transformation of data. The transformed data can be saved to storage generally, or in particular formats that enable the construction or depiction of a physical and tangible object. In some embodiments, the manipulation can be performed by a processor. In such an example, the processor thus transforms the data from one thing to another. Still further, some embodiments include methods can be processed by one or more machines or processors that can be coupled over a network. Each machine can transform data from one state or thing to another, and can also process data, save data to storage, transmit data over a network, display the result, or communicate the result to another machine. Computer-readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data.

Although method operations can be described in a specific order, it should be understood that other housekeeping operations can be performed in between operations, or operations can be adjusted so that they occur at slightly different times, or can be distributed in a system which allows the occurrence of the processing operations at various intervals associated with the processing, as long as the processing of the overlay operations are performed in the desired way.

It will be appreciated by those skilled in the art that while the invention has been described above in connection with particular embodiments and examples, the invention is not necessarily so limited, and that numerous some embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein. Various features and advantages of the invention are set forth in the following claims.

We claim:

1. A system for registering anatomical landmarks, comprising:
a two-part fiducial device including a top-half fiducial and a bottom-half fiducial, wherein the top-half fiducial includes a surface tracing registration groove and the bottom-half fiducial includes an adhesive backing; and
probe configured to engage the surface tracing registration groove to register the 3D location of the two-part fiducial device.

2. The system of claim 1, wherein the two-part fiducial is configured to be skin-mounted using the adhesive backing.

3. The system of claim 1, wherein the top-half fiducial contains an array of asymmetrically-arranged, embedded radiopaque fiducial markers.

4. The system of claim 3, wherein radiopaque markers are configured for X-ray based registration of a 3D location and pose of the two-part fiducial device relative to underlying anatomical landmarks of interest.

5. The system of claim 1, wherein the top-half fiducial and the bottom-half fiducial are two complementary halve configured to mate.

6. The system of claim 5, wherein a surgical drape is configured to be disposed between the top-half fiducial and the bottom-half fiducial when the top-half fiducial mates with the bottom-half fiducial.

7. The system of claim 1, wherein the top-half fiducial is configured to clamp with the bottom-half fiducial.

8. The system of claim 1, wherein the probe includes a probe shaft.

9. The system of claim 8, wherein the probe shaft is encased in a trigger sleeve handle.

10. The system of claim 9, wherein the probe shaft has a 3D-tracked dynamic reference frame.
